SEQUENCE LISTING (193 sequences)

SEQ ID NO: 1 - protein, 14 aa, artificial sequence
  TPO/mimetic peptide
IEGPTLRQWLAARA

SEQ ID NO: 2 - protein, 15 aa, artificial sequence
  TPO mimetic with flanking amino acids
IEGPTLRQWLAARAP

SEQ ID NO: 3 - protein, 18 aa, artificial sequence
  EPO mimetic peptide
DYHCRMGPLTWVCKPLGG

SEQ ID NO: 4 - protein, 16 aa, human
GDTIFGVTMGYYAMDV

SEQ ID NO: 5 - DNA, 24 bp, artificial sequence
  primer
tatcgcgattgcagtggcactggc

SEQ ID NO: 6 - DNA, 59 bp, artificial sequence
  primer
gccagccattgccgcagcgtcggcccttcaatynnynntctcgcacaataatatatggc

SEQ ID NO: 7 - DNA, 59 bp, artificial sequence
  primer
ccgacgctgcggcaatggctggcggcgcgcgcgnnynnytggggccaagggaccaccgt

SEQ ID NO: 8 - DNA, 22 bp, artificial sequence
  primer
tcaaaatcaccggaaccagagc

SEQ ID NO: 9 - DNA, 59 bp, artificial sequence
  primer
gccagccattgccgcagcgtcggcccttcaatnggnggtctcgcacaataatatatggc

SEQ ID NO: 10 - DNA, 59 bp, artificial sequence
  primer
ccgacgctgcggcaatggctggcggcgcgcgcgggnggntggggccaagggaccaccgt

SEQ ID NO: 11 - DNA, 59 bp, artificial sequence
  primer
gccagccattgccgcagcgtcggcccttcaatnccncctctcgcacaataatatatggc

SEQ ID NO: 12 - DNA, 59 bp, artificial sequence; identical to SEQ ID NO: 10
  primer

SEQ ID NO: 13 - DNA, 21 bp, artificial sequence
  primer
gctgcccaaccagccatggcc

SEQ ID NO: 14 - DNA, 60 bp, artificial sequence
  primer
ccaaccctgcgccagtggctggctgctcgcgctnnknnkagagtcaccattaccgcggac

SEQ ID NO: 15 - DNA, 24 bp, artificial sequence
  primer
agcgtagtccggaacgtcgtacgg

SEQ ID NO: 16 - DNA, 60 bp, artificial sequence
  primer
agccagccactggcgcagggttgggccttcgatmnnmnnacagtagtacactgcaaaatc

SEQ ID NO: 17 - DNA, 60 bp, artificial sequence
  primer
ccaaccctgcgccagtggctggctgctcgcgctnnknnkttcggccaagggaccaaggtg

SEQ ID NO: 18 - DNA, 21 bp, artificial sequence
  primer
ggccatggctggttgggcagc

SEQ ID NO: 19 - DNA, 60 bp, artificial sequence
  primer
agccagccactggcgcagggttgggccttcgatmnnmnnatagatgaggagcctgggagc

SEQ ID NO: 20 - DNA, 60 bp, artificial sequence
  primer
ccaaccctgcgccagtggctggctgctcgcgctnnknnkggcatcccagacaggttcagt

SEQ ID NO: 21 - DNA, 63 bp, artificial sequence
  primer
cacccaggtcagtgggcccatgcgmnnatgatagtcmnnmnntctcgcacaataatatatggc

SEQ ID NO: 22 - DNA, 63 bp, artificial sequence
  primer
cgcatgggcccactgacctgggtgnnkaaaccactgnnknnktggggccaagggaccacggtc

SEQ ID NO: 23 - DNA, 63 bp, artificial sequence
  primer
cacccaggtcagtgggcccatgcgmnnatgatagtcmnnmnnacagtagtacactgcaaaatc

SEQ ID NO: 24 - DNA, 63 bp, artificial sequence
  primer
cgcatgggcccactgacctgggtgnnkaaaccactgnnknnkttcggccaagggaccaaggtg

SEQ ID NO: 25 - protein, 18 aa, artificial sequence
  TPO mimetic with flanking amino acids
PPIEGPTLRQWLAARAGG

SEQ ID NO: 26 - DNA, 54 bp, artificial sequence
  nucleic acid encoding TPO mimetic with flanking amino acids
ccgcccattgaagggccgacgctgcggcaatggctggcggcgcgcgcgggaggc

SEQ ID NO: 27 - protein, 18 aa, artificial sequence
  TPO mimetic with flanking amino acids
GGIEGPTLRQWLAARAGG

SEQ ID NO: 28 - DNA, 54 bp, artificial sequence
  nucleic acid encoding TPO mimetic with flanking amino acids
gggggtattgaagggccgacgctgcggcaatggctggcggcgcgcgcgggcgga

SEQ ID NO: 29 - protein, 18 aa, artificial sequence
  TPO mimetic with flanking amino acids
GGIEGPTLRQWLAARAGG

SEQ ID NO: 30 - DNA, 54 bp, artificial sequence
  nucleic acid encoding TPO mimetic with flanking amino acids
ggcggtattgaagggccgacgctgcggcaatggctggcggcgcgcgcgggaggc

SEQ ID NO: 31 - protein, 18 aa, artificial sequence
  TPO mimetic with flanking amino acids
WLIEGPTLRQWLAARAPV

SEQ ID NO: 32 - DNA, 54 bp, artificial sequence
  nucleic acid encoding TPO mimetic with flanking amino acids
tggctgattgaagggccgacgctgcggcaatggctggcggcgcgcgcgcctgtc

SEQ ID NO: 33 - protein, 18 aa, artificial sequence
  TPO mimetic with flanking amino acids
MIIEGPTLRQWLAARAVG

SEQ ID NO: 34 - DNA, 54 bp, artificial sequence
  nucleic acid encoding TPO mimetic with flanking amino acids
atgataattgaagggccgacgctgcggcaatggctggcggcgcgcgcggttggc

SEQ ID NO: 35 - protein, 18 aa, artificial sequence
  TPO mimetic with flanking amino acids
VVIEGPTLRQWLAARAPV

SEQ ID NO: 36 - DNA, 54 bp, artificial sequence
  nucleic acid encoding TPO mimetic with flanking amino acids
gtggtaattgaagggccgacgctgcggcaatggctggcggcgcgcgcgcctgtt

SEQ ID NO: 37 - protein, 18 aa, artificial sequence
  TPO mimetic with flanking amino acids
GPIEGPTLRQWLAARAPD

SEQ ID NO: 38 - DNA, 54 bp, artificial sequence
  nucleic acid encoding TPO mimetic with flanking amino acids
gggccgattgaagggccgacgctgcggcaatggctggcggcgcgcgcgcccgat

SEQ ID NO: 39 - protein, 18 aa, artificial sequence
  TPO mimetic with flanking amino acids
LPIEGPTLRQWLAARAPV

SEQ ID NO: 40 - DNA, 54 bp, artificial sequence
  nucleic acid encoding TPO mimetic with flanking amino acids
ttgccaattgaagggccgacgctgcggcaatggctggcggcgcgcgcgcctgtt

SEQ ID NO: 41 - protein, 18 aa, artificial sequence
  TPO mimetic with flanking amino acids
SLIEGPTLRQWLAARAPI

SEQ ID NO: 42 - DNA, 54 bp, artificial sequence
  nucleic acid encoding TPO mimetic with flanking amino acids
tcactgattgaagggccgacgctgcggcaatggctggcggcgcgcgcgcccatc

SEQ ID NO: 43 - protein, 18 aa, artificial sequence
  TPO mimetic with flanking amino acids
TMIEGPTLRQWLAARAPV

SEQ ID NO: 44 - DNA, 54 bp, artificial sequence
  nucleic acid encoding TPO mimetic with flanking amino acids
acaatgattgaagggccgacgctgcggcaatggctggcggcgcgcgcgcccgtt

SEQ ID NO: 45 - protein, 18 aa, artificial sequence
  TPO mimetic with flanking amino acids
TTIEGPTLRQWLAARAPV

SEQ ID NO: 46 - DNA, 54 bp, artificial sequence
  nucleic acid encoding TPO mimetic with flanking amino acids
acgacaattgaagggccgacgctgcggcaatggctggcggcgcgcgcgcctgtc

SEQ ID NO: 47 - protein, 18 aa, artificial sequence
  TPO mimetic with flanking amino acids
TRIEGPTLRQWLAARACS

SEQ ID NO: 48 - DNA, 54 bp, artificial sequence
  nucleic acid encoding TPO mimetic with flanking amino acids
acacggattgaagggccgacgctgcggcaatggctggcggcgcgcgcgtgcagc

SEQ ID NO: 49 - protein, 18 aa, artificial sequence
  TPO mimetic with flanking amino acids
QTIEGPTLRQWLAARAPD

SEQ ID NO: 50 - DNA, 54 bp, artificial sequence
  nucleic acid encoding TPO mimetic with flanking amino acids
cagacaattgaagggccgacgctgcggcaatggctggcggcgcgcgcgcctcac

SEQ ID NO: 51 - DNA, 60 bp, artificial sequence
  primer
agccagccactggcgcagggttgggccttcgatmnnmnntcccatccactcaagcccttg

SEQ ID NO: 52 - DNA, 270 bp, artificial sequence
  portion of vector
actagtccgaaaccgtctaccccaccgggctcttcctgcggtggccgcatcgcccgtctggaggaaaaagtgaaaaccctgaaagctcagaactccgagctggcgtccactgccaacatgctgcgcgaacaggtggcacagctgaaacagaaagttatgaaccatggcggttgtgctagtggccaggccggccagcaccatcaccatcaccatggcgcatacccgtacgacgttccggactacgcttcttaggagggtggtggctctgag

SEQ ID NO: 53 - protein, 81 aa, artificial sequence
  portion of polypeptide encoded by Seq.52
PKPSTPPGSSCGGRIARLEEKVKTLKAQNSELASTANMLREQVAQLKQKVMNHGGCASGQAGQHHHHHHGAYPYDVPDYAS

SEQ ID NO: 54 - DNA, 699 bp, human
gaggtgcagctgctcgagcagtctggggctgaggtgaagaagcctgggtcctcggtgaaggtctcctgcagggcttctggaggcaccttcaacaattatgccatcagctgggtgcgacaggcccctggacaagggcttgagtggatgggagggatcttccctttccgtaatacagcaaagtacgcacaacacttccagggcagagtcaccattaccgcggacgaatccacgggcacagcctacatggagctgagcagcctgagatctgaggacacggccatatattattgtgcgagaggggatacgatttttggagtgaccatgggatactacgctatggacgtctggggccaagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagt

SEQ ID NO: 55 - DNA, 646 bp, human
gagctcacgcagtctccaggcaccctgtctttgtctccaggggaaagagccaccctctcctgcagggccagtcacagtgttagcagggcctacttagcctggtaccagcagaaacctggccaggctcccaggctcctcatctatggtacatccagcagggccactggcatcccagacaggttcagtggcagtgggtctgggacagacttcactctcaccatcagcagactggagcctgaagattttgcagtgtactactgtcagcagtatggtggctcaccgtggttcggccaagggaccaaggtggaactcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagcttgcccgtcacaaagagcttcaacaggggagagtgttagttctaga

SEQ ID NO: 56 - protein, 28 aa, artificial sequence
  portion of artificial heavy chain variable region
YYCARXXIEGPTLRQWLAARAXXWGQGT

SEQ ID NO: 57 - DNA, 84 bp, artificial sequence
  nucleotides encoding artificial CDR3
tattattgtgcgagannrnnrattgaagggccgacgctgcggcaatggctggcggcgcgcgcgnnynnytggggccaagggacc

SEQ ID NO: 58 - DNA, 60 bp, artificial sequence
  primer
ccaaccctgcgccagtggctggctgctcgcgctnnknnktggtaccagcagaaacctggc

SEQ ID NO: 59 - DNA, 60 bp, artificial sequence
  primer
agccagccactggcgcagggttgggccttcgatmnnmnngcaggagagggtggctctttc

SEQ ID NO: 60 - DNA, 5149 bp, artificial sequence
  vector
gggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagctgtacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattcaggaggaatttaaaatgaaaaagacagctatcgcgattgcagtggcactggctggtttcgctaccgtggcccaggcggccgagctcggccatggctggttgggcagcgagtaataacaatccagcggctgccgtaggcaataggtatttcattatgactgtctccttggcgactagctagtttagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgttactaatgatggtgatggtgatggctagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcgccggttcggggaagtagtccttgaccaggcagcccagggccgctgtgcccccagaggtgctcttggaggagggtgccagggggaagaccgatgggcccttggtggaggctgcggagacggtgaccgtggtaccagcagaaacctggccaggctcccaggctcctcatctatggtacatccagcagggccactggcatcccagacaggttcagtggcagtgggtctgggacagacttcactctcaccatcagcagactggagcctgaagattttgcagtgtactactgtcagcagtatggtggctcaccgtggttcggccaagggaccaaggtggaactcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacggaggagagtgttaattctagataattaattaggaggaatttaaaatgaaatacctattgcctacggcagccgctggattgttattactcgctgcccaaccagccatggccgaggtgcagctgctcgagatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcacttgtctaaaggtcagttgaaagagttcctcgacgctaacctggcgtacccgtacgacgttccggactacggttctactagtccgaaaccgtctaccccaccgggctcttcctgcggtggccgcatcgcccgtctggaggaaaaagtgaaaaccctgaaagctcagaactccgagctggcgtccactgccaacatgctgcgcgaacaggtggcacagctgaaacagaaagttatgaaccatggcggttgtgctagtggccaggccggccagcaccatcaccatcaccatggcgcatacccgtacgacgttccggactacgcttcttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaattaatttaagcggccgcagatct

SEQ ID NO: 61 - protein, 18 aa, artificial sequence
  TPO mimetic with flanking amino acids
NPIEGPTLRQWLAARARG

SEQ ID NO: 62 - DNA, 41 bp, artificial sequence
  primer
taggatgcggccgcacaggtctttttttttttttttttttt

SEQ ID NO: 63 - DNA, 24 bp, artificial sequence
  primer
ccatgtaggctgtgcccgtggatt

SEQ ID NO: 64 - DNA, 24 bp, artificial sequence
  primer
ccacgggcacagcctacatggagc

SEQ ID NO: 65 - DNA, 54 bp, artificial sequence; identical to SEQ ID NO: 40
  nucleic acid encoding TPO mimetic peptide flanking sequence

SEQ ID NO: 66 - protein, 18 aa, artificial sequence
  TPO mimetic peptide with flanking sequence
LPIEGPTLRQWLAARAPV

SEQ ID NO: 67 - protein, 472 aa, artificial sequence
  Humanized antibody heavy chain
MKWSWVILFLLSVTAGVHSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARLPIEGPTLRQWLAARAPVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - DNA, 1419 bp, artificial sequence
  nucleic acid encoding humanized antibody heavy chain
atgaagtggagctgggttattctcttcctcctgtcagtaactgccggcgtccactcccaagtccaactggtgcaatccggcgccgaggtcaagaagccaggggcctcagtcaaagtgtcctgtaaagctagcggctatattttttctaattattggattcaatgggtgcgtcaggcccccgggcagggcctggaatggatgggtgagatcttaccgggctctggtagcaccgaatataccgaaaattttaaagaccgtgttactatgacgcgtgacacttcgactagtacagtatacatggagctctccagcctgcgatcggaggacacggccgtctattattgcgcgcgtttgccaattgaagggccgacgctgcggcaatggctggcggcgcgcgcgcctgtttggggtcaaggaaccctggtcactgtctcgagcgcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 69 - protein, 236 aa, artificial sequence
  Humanized antibody light chain
MDMRVPAQLLGLLLLWLRGARCDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - DNA, 711 bp, artificial sequence
  nucleic acid encoding humanized antibody light chain
atggacatgagggtccccgctcagctcctggggctcctgctactctggctccgaggtgccagatgtgatatccagatgacccagtccccgtcctccctgtccgcctctgtgggcgatagggtcaccatcacctgcggcgccagcgaaaacatctatggcgcgctgaactggtatcaacagaaacccgggaaagctccgaagcttctgatttacggtgcgacgaacctggcagatggagtcccttctcgcttctctggatccggctccggaacggatttcactctgaccatcagcagtctgcagcctgaagacttcgctacgtattactgtcagaacgttttaaatactccgttgactttcggacagggtaccaaggtggaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 71 - protein, 22 aa, artificial sequence
  EPO mimetic with random flanking amino acids
XXDYHXRMGPLTWVXKPLGGXX

SEQ ID NO: 72 - DNA, 21 bp, artificial sequence
  primer
taggatgcggccgcacaggtc

SEQ ID NO: 73 - DNA, 39 bp, artificial sequence
  primer
cacgcgcacaacacgtctagaracatccagatgacccag

SEQ ID NO: 74 - DNA, 39 bp, artificial sequence
  primer
cacgcgcacaacacgtctagagmcatccagttgacccag

SEQ ID NO: 75 - DNA, 39 bp, artificial sequence
  primer
cacgcgcacaacacgtctagagccatccrgatgacccag

SEQ ID NO: 76 - DNA, 39 bp, artificial sequence
  primer
cacgcgcacaacacgtctagagtcatctggatgacccag

SEQ ID NO: 77 - DNA, 39 bp, artificial sequence
  primer
cacgcgcacaacacgtctagagatattgtgatgacccag

SEQ ID NO: 78 - DNA, 39 bp, artificial sequence
  primer
cacgcgcacaacacgtctagagatrttgtgatgactcag

SEQ ID NO: 79 - DNA, 39 bp, artificial sequence
  primer
cacgcgcacaacacgtctagagaaattgtgttgacrcag

SEQ ID NO: 80 - DNA, 39 bp, artificial sequence
  primer
cacgcgcacaacacgtctagagaaatagtgatgacgcag

SEQ ID NO: 81 - DNA, 39 bp, artificial sequence
  primer
cacgcgcacaacacgtctagagaaattgtaatgacacag

SEQ ID NO: 82 - DNA, 39 bp, artificial sequence
  primer
cacgcgcacaacacgtctagagacatcgtgatgacccag

SEQ ID NO: 83 - DNA, 39 bp, artificial sequence
  primer
cacgcgcacaacacgtctagagaaacgacactcacgcag

SEQ ID NO: 84 - DNA, 39 bp, artificial sequence
  primer
cacgcgcacaacacgtctagagaaattgtgctgactcag

SEQ ID NO: 85 - DNA, 39 bp, artificial sequence
  primer
cacgcgcacaacacgtctagagatgttgtgatgacacag

SEQ ID NO: 86 - DNA, 22 bp, artificial sequence
  primer
attaatacgactcactataggg

SEQ ID NO: 87 - DNA, 20 bp, artificial sequence
  primer
aattaaccctcactaaaggg

SEQ ID NO: 88 - DNA, 59 bp, artificial sequence
  primer
agccagccactggcgcagggttgggccttcgatcgggttcctgatgaggagctttggrg

SEQ ID NO: 89 - DNA, 59 bp, artificial sequence
  primer
agccagccactggcgcagggttgggccttcgatcgggttttgaataatgaaaatagcag

SEQ ID NO: 90 - DNA, 59 bp, artificial sequence
  primer
agccagccactggcgcagggttgggccttcgatcgggttgtaaatgagcarcttaggag

SEQ ID NO: 91 - DNA, 59 bp, artificial sequence
  primer
agccagccactggcgcagggttgggccttcgatcgggttatagatgaggagcctgggmg

SEQ ID NO: 92 - DNA, 59 bp, artificial sequence
  primer
agccagccactggcgcagggttgggccttcgatcgggttataaattaggcgccttggag

SEQ ID NO: 93 - DNA, 59 bp, artificial sequence
  primer
agccagccactggcgcagggttgggccttcgatcgggttatagatyaggagctgtggag

SEQ ID NO: 94 - DNA, 58 bp, artificial sequence
  primer
agccagccactggcgcagggttgggccttcgatcgggttatagatcaggagcttagga

SEQ ID NO: 95 - DNA, 58 bp, artificial sequence
  primer
agccagccactggcgcagggttgggccttcgatcgggttrtagatcaggagcttaggg

SEQ ID NO: 96 - DNA, 58 bp, artificial sequence
  primer
agccagccactggcgcagggttgggccttcgatcgggttatagatcagggacttaggg

SEQ ID NO: 97 - DNA, 58 bp, artificial sequence
  primer
agccagccactggcgcagggttgggccttcgatcgggttatagatcaggygcttaggg

SEQ ID NO: 98 - DNA, 59 bp, artificial sequence
  primer
ccaaccctgcgccagtggctggctgctcgcgctcgtggtggggtcccctcgaggttcag

SEQ ID NO: 99 - DNA, 59 bp, artificial sequence
  primer
ccaaccctgcgccagtggctggctgctcgcgctcgtggtggaatcccacctcgattcag

SEQ ID NO: 100 - DNA, 59 bp, artificial sequence
  primer
ccaaccctgcgccagtggctggctgctcgcgctcgtggtggggtccctgaccgattcag

SEQ ID NO: 101 - DNA, 59 bp, artificial sequence
  primer
ccaaccctgcgccagtggctggctgctcgcgctcgtggtggcatcccagmcaggttcag

SEQ ID NO: 102 - DNA, 59 bp, artificial sequence
  primer
ccaaccctgcgccagtggctggctgctcgcgctcgtggtggtatcccagccaggttcag

SEQ ID NO: 103 - DNA, 59 bp, artificial sequence
  primer
ccaaccctgcgccagtggctggctgctcgcgctcgtggtggagtsccagayaggttcag

SEQ ID NO: 104 - DNA, 59 bp, artificial sequence
  primer
ccaaccctgcgccagtggctggctgctcgcgctcgtggtggggtcccwgacagrttcag

SEQ ID NO: 105 - DNA, 59 bp, artificial sequence
  primer
ccaaccctgcgccagtggctggctgctcgcgctcgtggtggggtcccatcaaggttcag

SEQ ID NO: 106 - DNA, 59 bp, artificial sequence
  primer
ccaaccctgcgccagtggctggctgctcgcgctcgtggtggggtcccatctcggttcag

SEQ ID NO: 107 - DNA, 102 bp, artificial sequence
  oligonucleotide
aattcaaggagttaattatgaaaaaaaccgcgattgcgattgcggtggcgctggcgggctttgcgaccgtggcccaggcggcctctagaatctgcggccgca

SEQ ID NO: 108 - DNA, 102 bp, artificial sequence
  oligonucleotide
ctagtgcggccgcagattctagaggccgcctgggccacggtcgcaaagcccgccagcgccaccgcaatcgcaatcgcggtttttttcataattaactccttg

SEQ ID NO: 109 - DNA, 36 bp, artificial sequence
  primer
ggagtctagataactgtggctgcaccatctgtcttc

SEQ ID NO: 110 - DNA, 37 bp, artificial sequence
  primer
aggagcggccgcttaacactctcccctgttgaagctc

SEQ ID NO: 111 - DNA, 4883 bp, artificial sequence
  vector
gggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagctgtacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattcaaggagttaattatgaaaaaaaccgcgattgcgattgcggtggcgctggcgggctttgcgaccgtggcccaggcggcctctagataactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcactagatataattaaggagataaatatgaaatatctgctgccgaccgcggcggcgggcctgctgctgctggcggcgcagccggcgatggcgctcgagctgatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtggccaggccggccagcaccatcaccatcaccatggcgcatacccgtacgacgttccggactacgcttcttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaattaatttaagcggccggccgcagatctgctctctgaggaggatct

SEQ ID NO: 112 - protein, 8 aa, artificial sequence
  part of mimetic
GPTLRQWL

SEQ ID NO: 113 - protein, 18 aa, artificial sequence
  artificial CDR2
GXGPTLRQWLXYAQKFQG

SEQ ID NO: 114 - DNA, 48 bp, artificial sequence
  primer
cagccactggcgcagggttgggccmnnccctcccatccactcaagccc

SEQ ID NO: 115 - DNA, 60 bp, artificial sequence
  primer
ggcccaaccctgcgccagtggctgnnktacgcacagaaattccagggcagagtcaccatt

SEQ ID NO: 116 - DNA, 354 bp, artificial sequence
  nucleotides encoding variable region of light chain
gacatccagatgacccagtctccatcctccctgtctgcatctgtaggagacagagtcaccatcacttgccgggcgagtcagagtattagtagtttgctggcctggtatcagcagaaaccagggaaagctcctaagctcctgatctataacccgatcgaaggcccaaccctgcgccagtggctggctactcgcgctcgtggtggggtcccatcaaggttcagcggcagtggatctgggacagatttcactctcaccatcagcagcctgcagcctgaagattttgcaacttattactgccaacagtataatagttaccctcccactttcggccctgggaccaaagtggatatcaaa

SEQ ID NO: 117 - protein, 233 aa, human
EVQLLEQSGAEVKKPGSSVKVSCRASGGTFNNYAISWVRQAPGQGLEWMGGIFPFRNTAKYAQHFQGRVTITADESTGTAYMELSSLRSEDTAIYYCARGDTIFGVTMGYYAMDVWGQGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTS

SEQ ID NO: 118 - protein, 212 aa, human
ELTQSPGTLSLSPGERATLSCRASHSVSRAYLAWYQQKPGQAPRLLIYGTSSRATGIPDRFSGSGSGTDFTLTISRLEPEDFAVYYCQQYGGSPWFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 119 - protein, 22 aa, artificial sequence
  TPO mimetic peptide
GIFXXIEGPTLRQWLAARAXXG

SEQ ID NO: 120 - DNA, 60 bp, artificial sequence
  primer
agccagccactggcgcagggttgggccttcgatmnnmnngaagatccctcccatccactc

SEQ ID NO: 121 - DNA, 60 bp, artificial sequence
  primer
ccaaccctgcgccagtggctggctgctcgcgctnnknnkggcagagtcaccattaccgcg

SEQ ID NO: 122 - protein, 215 aa, artificial sequence
  antibody light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 123 - protein, 108 aa, artificial sequence
  antibody light chain variable region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 124 - protein, 249 aa, artificial sequence
  antibody heavy chain
QVQLVQSGAEVKKPGSSVKVSCKASGGTFSSYAISWVRQAPGQGLEWMGQLIEGPTLRQWLAARANSRVTITADESTSTAYMELSSLRSEDTAVYYCARLPIEGPTLRQWLAARAPVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGP

SEQ ID NO: 125 - protein, 128 aa, artificial sequence
  antibody heavy chain variable region
QVQLVQSGAEVKKPGSSVKVSCKASGGTFSSYAISWVRQAPGQGLEWMGQLIEGPTLRQWLAARANSRVTITADESTSTAYMELSSLRSEDTAVYYCARLPIEGPTLRQWLAARAPVWGQGTTVTVSS

SEQ ID NO: 126 - protein, 22 aa, artificial sequence
  heavy chain CDR2 clone
GIFXXIEGPTLRQWLAARAXXG

SEQ ID NO: 127 - protein, 22 aa, artificial sequence
  heavy chain CDR2 clone
GIFSPIEGPTLRQWLAARAAGG

SEQ ID NO: 128 - protein, 22 aa, artificial sequence
  heavy chain CDR2 clone
GIFPQIEGPTLRQWLAARAKHG

SEQ ID NO: 129 - protein, 22 aa, artificial sequence
  heavy chain CDR2 clone
GIFPNIEGPTLRQWLAARATGG

SEQ ID NO: 130 - protein, 22 aa, artificial sequence
  heavy chain CDR2 clone
GIFKGIEGPTLRQWLAARAPGG

SEQ ID NO: 131 - protein, 22 aa, artificial sequence
  heavy chain CDR2 clone
GIFPPIEGPTLRQWLAARAAVG

SEQ ID NO: 132 - protein, 22 aa, artificial sequence
  heavy chain CDR2 clone
GIFPRIEGPTLRQWLAARAKLG

SEQ ID NO: 133 - protein, 22 aa, artificial sequence
  heavy chain CDR2 clone
GIFPRIEGPTLRQWLAARAKLG

SEQ ID NO: 134 - protein, 22 aa, artificial sequence
  heavy chain CDR2 clone
GIFPYIEGPTLRQWLAARAKRG

SEQ ID NO: 135 - DNA, 42 bp, artificial sequence
  primer
gacgcgcacaacacggagctcgaaattgtgctgacccagagc

SEQ ID NO: 136 - DNA, 44 bp, artificial sequence
  primer
agacagtgagcgccgtctagaattagcattcgccgcggttaaag

SEQ ID NO: 137 - DNA, 36 bp, artificial sequence
  primer
gacgcgcacaacacgggcccgagcgtgtttccgctg

SEQ ID NO: 138 - DNA, 41 bp, artificial sequence
  primer
agacagtgagcgccgactagttttatcgcagcttttcggtt

SEQ ID NO: 139 - DNA, 41 bp, artificial sequence
  primer
gagccgcacgagcccctcgagcaggtgcagctggtgcagag

SEQ ID NO: 140 - DNA, 35 bp, artificial sequence
  primer
gcaaagtgtgaggggcccttggtgctcgcgctgct

SEQ ID NO: 141 - DNA, 4793 bp, artificial sequence
  expression vector
gggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagctgtacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattcaggaggaatttaaaatgaaaaagacagctatcgcgattgcagtggcactggctggtttcgctaccgtggcccaggcggccgagctcgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggtagcggcagcggcaccgattttaccctgaccattagccgcctggaaccggaagattttgcggtgtattattgccagcagtatggcagcagcccgtggacctttggccagggcaccaaagtggaaattaaacgcaccgtggcggcaccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcctgccggtgaccaaaagctttaaccgcggcgaatgctaattctagataattaattaggaggaatttaaaatgaaatacctattgcctacggcagccgctggattgttattactcgctgcccaaccagccatggccctcgagcaggtgcagctggtgcagagcggcgcggaagtgaaaaaaccgggcagcagcgtgaaagtgagctgcaaagcgagcggcggcacctttagcagctatgcgattagctgggtgcgccaggcgccgggccagggcctggaatggatgggccagctgattgaaggcccgaccctgcgccagtggctggcggcgcgcgcgaacagccgcgtgaccattaccgcggatgaaagcaccagcaccgcgtatatggaactgagcagcctgcgcagcgaagataccgcggtgtattattgcgcgcgcctgccgattgaaggcccgaccctgcgccagtggctggcggcgcgcgcgccggtgtggggccagggcaccaccgtgaccgtgagcagcgcgagcaccaagggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctatatagcctgagcagcgtggtgaccgtgccgagcagcagcctgggcacccagacctatatttgcaacgtgaaccataaaccgagcaacaccaaagtggataaaaaagtggaaccgaaaagctgcgataaaactagtggccaggccggccagcaccatcaccatcaccatggcgcatacccgtacgacgttccggactacgcttcttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaattaatttaagcggccgcagatctgctctctgaggaggatct

SEQ ID NO: 142 - protein, 239 aa, artificial sequence
  recombinant Ab light chain
MKKTAIAIAVALAGFATVAQAAELEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 143 - protein, 282 aa, artificial sequence
  recombinant Ab heavy chain
MKYLLPTAAAGLLLLAAQPAMALEQVQLVQSGAEVKKPGSSVKVSCKASGGTFSSYAISWVRQAPGQGLEWMGQLIEGPTLRQWLAARANSRVTITADESTSTAYMELSSLRSEDTAVYYCARLPIEGPTLRQWLAARAPVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGQAGQHHHHHHGAYPYDVPDYAS

SEQ ID NO: 144 - DNA, 24 bp, artificial sequence
  primer
aaaggtgccgccgctcgctttgca

SEQ ID NO: 145 - DNA, 42 bp, artificial sequence
  primer
ggcggcacctttnnknnktatgcgattagctgggtgcgccag

SEQ ID NO: 146 - DNA, 42 bp, artificial sequence
  primer
ggcggcacctttaacaactatgcgattagctgggtgcgccag

SEQ ID NO: 147 - protein, 128 aa, artificial sequence; identical to SEQ ID NO: 125
  cloned antibody VH

SEQ ID NO: 148 - protein, 128 aa, artificial sequence
  cloned antibody VH
QVQLVQSGAEVKKPGSSVKVSCKASGGTFNNYAISWVRQAPGQGLEWMGQLIEGPTLRQWLAARANSRVTITADESTSTAYMELSSLRSEDTAVYYCARLPIEGPTLRQWLAARAPVWGQGTTVTVSS

SEQ ID NO: 149 - protein, 128 aa, artificial sequence
  cloned antibody VH
QVQLVQSGAEVKKPGSSVKVSCKASGGTFGEYAISWVRQAPGQGLEWMGQLIEGPTLRQWLAARANSRVTITADESTSTAYMELSSLRSEDTAVYYCARLPIEGPTLRQWLAARAPVWGQGTTVTVSS

SEQ ID NO: 150 - protein, 128 aa, artificial sequence
  cloned antibody VH
QVQLVQSGAEVKKPGSSVKVSCKASGGTFQDYAISWVRQAPGQGLEWMGQLIEGPTLRQWLAARANSRVTITADESTSTAYMELSSLRSEDTAVYYCARLPIEGPTLRQWLAARAPVWGQGTTVTVSS

SEQ ID NO: 151 - protein, 128 aa, artificial sequence
  cloned antibody VH
QVQLVQSGAEVKKPGSSVKVSCKASGGTFPRYAISWVRQAPGQGLEWMGQLIEGPTLRQWLAARANSRVTITADESTSTAYMELSSLRSEDTAVYYCARLPIEGPTLRQWLAARAPVWGQGTTVTVSS

SEQ ID NO: 152 - protein, 18 aa, artificial sequence
  flanked TPO mimetic
QLIEGPTLRQWLAARANS

SEQ ID NO: 153 - DNA, 871 bp, human
gcaggatttagggcttggtctctcagcatcccacacttgtacagctgatgtggcatctgtgttttctttctcatcgtagatcaggctttgagctgtgaaataccctgcctcatgcatatgcaaataacctgaggtcttctgagataaatatagatatattggtgccctgagagcatcacataacaaccacattcctcctctaaagaagcccctgggagcacagctcatcaccatggactggacctggaggttcctctttgtggtggcagcgctacaggtaaggggcttcctagtcctaaggctgaggaagggatcctggtttagttaaagaggattttattcacccctgtgtcctctccacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagacacagtgtgaaaacccacatcctgagagtgtcagaaaccctgagggagaaggcagctgtgccgggctgaggagatgacagggtttattaggtttaaggctgtttacaaaatgggttatatatttgagaaaaaaagaacagtagaaacaagtacatactcctctaattttaagataattattccattcaagagtcgtaatat

SEQ ID NO: 154 - protein, 20 aa, human
YYYYYGMDVWGQGTTVTVSS

SEQ ID NO: 155 - DNA, 711 bp, human
cagctgctttgcatgtccctcccagccgccctgcagtccagagcccatatcaatgcctgggtcagagctctggagaagagctgctcagttaggacccagagggaaccatggaaaccccagcgcagcttctcttcctcctgctactctggctcccaggtgaggggaacatgggatggttttgcatgtcagtgaaaaccctctcaagtcctgttacctggcaactctgctcagtcaatacaataattaaagctcaatataaagcaataattctggctcttctgggaagacaatgggtttgatttagattacatgggtgacttttctgttttatttccaatctcagataccaccggagaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcccacagtgattcagcttgaaacaaaaacctctgcaagaccttcattgtttactagattataccagctg

SEQ ID NO: 156 - protein, 12 aa, human
WTFGQGTKVEIK

SEQ ID NO: 157 - DNA, 447 bp, artificial sequence
  recombinant DNA encoding AB VH
atgaaatacctattgcctacggcagccgctggattgttattactcgctgcccaaccagccatggcgcaggtgcagctggtgcagagcggcgcggaagtgaaaaaaggcagcagcgtgaaagtgagctgcaaagcgagcggcggcacctttagcagctatgcgattagctgggtgcgccaggcgccgggccagggcctggaatggatgggccagctgattgaaggcccgaccctgcgccagtggctggcggcgcgcgcgaacagccgcgtgaccattaccgcggatgaaagcaccagcaccgcgtatatggaactgagcagcctgcgcagcgaagataccgcggtgtattattgcgcgcgcctgccgattgaaggcccgaccctgcgccagtggctggcggcgcgcgcgccggtgtggggccagggcaccaccgtgaccgtgagcagc

SEQ ID NO: 158 - protein, 150 aa, artificial sequence
  recombinant Ab VH
MKYLLPTAAAGLLLLAAQPAMAQVQLVQSGAEVKKPGSSVKVSCKASGGTFSSYAISWVRQAPGQGLEWMGQLIEGPTLRQWLAARANSRVTITADESTSTAYMELSSLRSEDTAVYYCARLPIEGPTLRQWLAARAPVWGQGTTVTVSS

SEQ ID NO: 159 - protein, 127 aa, artificial sequence
  recombinant Ab VH
QVQLVQSGAEVKKPGSSVKVSCRASGGTFNNYAISWVRQAPGQGLEWMGGIFPFRNTAKYAQHFQGRVTITADESTGTAYMELSSLRSEDTAIYYCARLPIEGPTLRQWLAARAPVWGQGTTVTVSA

SEQ ID NO: 160 - DNA, 390 bp, artificial sequence
  recombinant DNA encoding Ab Vk
atgaaatacctattgcctacggcagccgctggattgttattactcgctgcccaaccagccatggcggaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagcccgtggacctttggccagggcaccaaagtggaaattaaa

SEQ ID NO: 161 - protein, 130 aa, artificial sequence
  recombinant Ab Vk
MKYLLPTAAAGLLLLAAQPAMAEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 162 - protein, 107 aa, artificial sequence
  recombinant Ab Vk
EIVLTQSPGTLSLSPGERATLSCRASHSVSRAYLAWYQQKPGQAPRLLIYGTSSRATGIPDRFSGSGSGTDFTLTISRLEPEDFAVYYCQQYGGSPWFGQGTKVELK

SEQ ID NO: 163 - DNA, 272 bp, artificial sequence
  primer
ccagccatggcgcaggtgcagctggtgcagagcggcgcggaagtgaaaaaaccgggcagcagcgtgaaagtgagctgcaaagcgagcggcggcacctttagcagctatgcgattagctgggtgcgccaggcgccgggccagggcctggaatggatgggcggcattattccgatttttggcaccgcgaactatgcgcagaaatttcagggccgcgtgaccattaccgcggatgaaagcaccagcaccgcgtatatggaactgagcagcctgcg

SEQ ID NO: 164 - DNA, 271 bp, artificial sequence
  primer
gttccagctcacggtcaccggttccggaaaataatctttcaccaggcagcccagcgccgcggtgccgccgctggtgcttttgctgctcggcgccagcggaaacacgctcgggcctttggtgctcgcgctgctcacggtcacggtggtgccctggccccacaccggcgcgcgcgccgccagccactggcgcagggtcgggccttcaatcggcaggcgcgcgcaataatacaccgcggtatcttcgctgcgcaggctgctcagttccatatac

SEQ ID NO: 165 - DNA, 274 bp, artificial sequence
  primer
cgagtctagattacgggccgcccagcagttccggcgccgggcacggcgggcaggtatgggttttatcgcagcttttcggttccacttttttatccactttggtgttgctcggtttatggttcacgttgcaaatataggtctgggtgcccaggctgctgctcggcacggtcaccacgctgctcaggctatacaggccgctgctctgcagcaccgccggaaaggtatgcacgccgctggtcagcgcgccgctgttccagctcacggtcaccggttc

SEQ ID NO: 166 - DNA, 236 bp, artificial sequence
  primer
ccagccatggcggaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgac

SEQ ID NO: 167 - DNA, 238 bp, artificial sequence
  primer
ctttcgcttcgcgcggataaaagttgttcagcaggcacaccacgctcgcggtgccgcttttcagtgttcatcgctcggcggaaaaataaacacgctcggcgccgccacggtgcgtttaatttccactttggtgccctggccaaaggtccacgggctgctgccatactgctggcaataatacaccgcaaaatcttccggttccaggcggctaatggtcagggtaaaatcggtgccgctg

SEQ ID NO: 168 - DNA, 245 bp, artificial sequence
  primer
gtgctgatcattagcattcgccgcggttaaagcttttggtcaccggcaggctcaggccctgatgggtcacttcgcacgcatacactttatgtttttcataatccgctttgctcagggtcagggtgctgctcaggctataggtgctatctttgctatcctgttcggtcacgctttcctggctgttgccgctctgcagcgcgttatccactttccactgcactttcgcttcgcgcggataaaagttg

SEQ ID NO: 169 - protein, 98 aa, artificial sequence
  recombinant Ab VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 170 - protein, 96 aa, human
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 171 - protein, 177 aa, artificial sequence
  recombinant Ab heavy chain
EGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 172 - protein, 17 aa, human
CFGRKMDRISSSSGLGC

SEQ ID NO: 173 - protein, 39 aa, human
HGEGRFTSDLSKQMEEEAVRLFIEWLKNGGPSSGAPPPS

SEQ ID NO: 174 - protein, 30 aa, human
HAEGTFTSDVSSYLEGQAAKEFIAWLVKGR

SEQ ID NO: 175 - protein, 34 aa, human
HADGSFSDEMNTILDNLAARDFINWLIQTKITDR

SEQ ID NO: 176 - protein, 29 aa, human
HSQGTFTSDYSKYLDSRRAQDRVQWLMNT

SEQ ID NO: 177 - protein, 38 aa, human
HSDGIFTDSYSRYRKQMAVKKYLAAVLGKRYKQRVKNK

SEQ ID NO: 178 - protein, 130 aa, human
FSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 179 - DNA, 21 bp, artificial sequence
  primer
gctgcccaaccagccatggcc

SEQ ID NO: 180 - DNA, 23 bp, artificial sequence
  primer
atcaaaatcaccggaaccagagc

SEQ ID NO: 181 - DNA, 72 bp, artificial sequence
  primer
ttccaaataagaacttacatcactggtaaaggtcccttcagcatgmnnmnntctcgcacaataatatatggc

SEQ ID NO: 182 - DNA, 72 bp, artificial sequence
  primer
ggccaagctgccaaggaattcattgcttggctggtgaaaggccgannknnktggggccaagggaccacggtc

SEQ ID NO: 183 - DNA, 50 bp, artificial sequence
  primer
cagtgatgtaagttcttatttggaaggccaagctgccaaggaattcattg

SEQ ID NO: 184 - DNA, 50 bp, artificial sequence
  primer
caatgaattccttggcagcttggccttccaaataagaacttacatcactg

SEQ ID NO: 185 - DNA, 24 bp, artificial sequence
  primer
tatgccatcagctgggtgcgacag

SEQ ID NO: 186 - DNA, 48 bp, artificial sequence
  primer
tcgcacccagctgatggcatamnnmnngaaggtgcctccagaagccct

SEQ ID NO: 187 - DNA, 23 bp, artificial sequence
  primer
atcaaaatcaccggaaccagagc

SEQ ID NO: 188 - DNA, 65 bp, artificial sequence
  primer
ctctgggctccaatcctgtccatcctgcccccgaagcamnnmnntctcgcacaataatatatggc

SEQ ID NO: 189 - DNA, 65 bp, artificial sequence
  primer
ggatggacaggattggagcccagagcggactgggctgtnnknnktggggccaagggaccacggtc

SEQ ID NO: 190 - DNA, 65 bp, artificial sequence
  primer
ctggaggagctgatccggtccatcttcctcccaaagcamnnmnntctcgcacaataatatatggc

SEQ ID NO: 191 - DNA, 65 bp, artificial sequence
  primer
agatggaccggatcagctcctccagtggcctgggctgcnnknnktggggccaagggaccacggtc

SEQ ID NO: 192 - DNA, 24 bp, artificial sequence
  primer
tatgccatcagctgggtgcgacag

SEQ ID NO: 193 - DNA, 48 bp, artificial sequence; identical to SEQ ID NO: 186
  primer

(19) United States
(12) Patent Application Publication
(10) Pub. No.: US 2004/0253242 A1
(43) Pub. Date: Dec. 16, 2004

(54) Rationally designed antibodies

(76) Inventors: Katherine S. Bowdish, Del Mar, CA (US); Shana Frederickson, Solana Beach, CA (US); Mark Renshaw, Solana Beach, CA (US); Cecelia Orencia, Del Mar, CA (US)

Correspondence Address: Mark Farber, C/O, Alexion Pharmaceuticals, Inc., 352 Knotter Drive, Cheshire, CT, 06410, US

(21) Appl. No.: 10/737,290

(22) Filed: Dec. 15, 2003

Related U.S. Application Data
(63) Continuation-in-part of application No. 10/452,590, filed on Jun. 2, 2003.
Application No. 10/452,590 is a continuation-in-part of application No. 10/307,724, filed on Dec. 2, 2002.
Application No. 10/307,724 is a continuation-in-part of application No. 10/006,593, filed on Dec. 5, 2001.
(60) Provisional application No. 60/251,448, filed on Dec. 5, 2000.
(60) Provisional application No. 60/288,889, filed on May 4, 2001.
(60) Provisional application No. 60/294,068, filed on May 29, 2001.

(51) Int. Cl. A61K 39/395; C07K 16/26

(52) U.S. Cl. 424/145.1; 530/388.24

(57) ABSTRACT

Antibodies or fragments thereof having CDR regions replaced or fused with biologically active peptides are described. Flanking sequences may optionally be attached at one or both the carboxy-terminal and amino-terminal ends of the peptide in covalent association with adjacent framework regions. Compositions containing such antibodies or fragments thereof are useful in therapeutic and diagnostic modalities.

### RELATED APPLICATIONS

This application is a continuation-in-part of U.S. application Ser. No. 10/452,590 filed Jun. 2, 2003, which is a continuation-in-part of U.S. application Ser. No. 10/307,724 filed Dec. 2, 2002 which is a continuation-in-part of U.S. application Ser. No.10/006,593 filed Dec. 5, 2001 which claims priority to U.S. Provisional Patent Application No. 60/251,448 filed Dec. 5, 2000, and to U.S. Provisional Patent Application No. 60/288,889 filed May 4, 2001, and to U.S. Provisional Patent Application No. 60/294,068 filed May 29, 2001. The disclosures of these three provisional applications are incorporated herein in their entirety by this reference.

### TECHNICAL FIELD

The present disclosure relates to antibody molecules and biologically active peptides as diagnostic and therapeutic reagents.

### BACKGROUND OF RELATED ART

Antibodies are produced by B lymphocytes and defend against infection. Antibodies are produced in millions of forms, each with a different amino acid sequence. Antibody molecules are composed of two identical light chains and two identical heavy chains. When digested by the enzyme papain, two identical Fab fragments are produced along with one Fc fragment. When digested with the enzyme pepsin one F(ab′)₂ fragment is produced. Light and heavy chains consist of constant and variable regions. Within the variable regions are hypervariable regions (aka complementarity determining regions (CDRs)) which form the antigen binding site. The remaining parts of the variable regions are referred to as framework regions.

Important biological functions, such as receptor binding, activation and enzymatic activity, are often attributable to discrete regions of larger protein molecules, comprising a limited number of amino acid residues. Peptides displaying binding, activation or enzymatic activity have also been discovered by screening libraries of peptides generated by the random linking of amino acid residues. These peptides may not correspond to a linear arrangement of amino acids in a larger protein molecule exhibiting similar biological activity and are referred to as discontinuous peptide epitopes or mimotopes. Certain peptide mimetics have been described and cloned. See, e.g., U.S. Pat. No. 6,083,913 (thrombopoietin (TPO) mimetic), U.S. Pat. No. 5,835,382 (erythropoietin (EPO) mimetic), U.S. Pat. No. 5,830,851 (EPO mimetic) and Wrighton et al, Science, (1996) 273:458-63. Peptide epitopes and mimotopes due to their small size are potentially advantageous over large protein molecules for use as therapeutic reagents. However, the results with these peptides as therapeutics may often be unsatisfactory. One drawback to the use of peptides as therapeutic reagents is that they are generally unstable in vivo, i.e., their clearance rates from serum may be quite rapid. In addition, it is difficult to predict the activity, therapeutic or otherwise, of a peptide if it is fused into a larger molecule since conformational changes and other molecular forces may interfere with or totally negate the activity of the peptide. Attempts have been made to introduce certain polypeptides into CDR regions of antibodies. See, e.g., PCT Appln. WO 94/18221. However, as mentioned previously, due to conformational changes which may be caused by surrounding amino acids, the biological activity of active polypeptides may be diminished or negated. Therefore, it is an object herein to provide rationally designed antibodies or fragments thereof which include biologically active peptides for use as diagnostic and therapeutic reagents.

### SUMMARY

Provided herein are biologically active recombinant antibodies and fragments thereof that mimic the activity of biologically active peptides, methods of making such antibodies and methods for their use in therapy and diagnosis. These antibodies and fragments thereof do not suffer from some of the disadvantages of isolated peptides, as antibodies naturally have long serum half-lives and are highly specific in binding their target. It has surprisingly been found that incorporation of particular amino acids surrounding a target peptide that has been combined into an antibody molecule actually increases the biological activity of the peptide.

Immunoglobulins or fragments thereof have a peptide of interest inserted into a complementarity determining region (CDR) of an antibody molecule. The antibody molecule serves as a scaffold for presentation of the peptide and confers upon the peptide enhanced stability. The peptide optionally replaces all the amino acids of a CDR region, or may be added to an existing CDR, whereby the original antigen specificity is disrupted, wherein the CDR region is defined by either of the two accepted schemes (See, Kabat et al., Sequences of Proteins of Immunologics Interest, 5^{th} ed (1991), NIH Publication 91-3242 and Chothia et al. J. Mol. Bio (1992) (227)776-98.) Furthermore, additional amino acids may be randomly introduced which flank the peptide and allow for the screening of optimum peptide presentation in the antibody framework. It has been surprisingly found that in certain cases a proline flanking the peptide provides an increase in biological activity.

In particular embodiments an immunoglobulin molecule or fragment has amino acids residues corresponding to one complementarity determining region (CDR) replaced with amino acid residues comprising a biologically active hemopoietic or thrombopoietic peptide. In another particular embodiment, amino acid residues corresponding to at least two complementarity determining regions (CDRs) are each replaced by amino acid residues comprising such a biologically active peptide. In a single immunoglobulin molecule or fragment thereof, one or more complementarity determining regions can be replaced with a peptide; for example, CDR3 of a heavy chain, CDR3 of a light chain, CDR3 of both a heavy and light chain, CDR2 and CDR3 of a heavy chain, or CDR2 and CDR3 of a light chain. Other combinations of replaced CDR regions are possible, including the replacement of CDR1. In addition, instead of replacement of a CDR, one could add the peptide to a native CDR without actual replacement of amino acid residues while still disrupting the original antigen specificity.

Thus, in one aspect, a biologically active peptide is provided with enhanced activity by adding a proline to its carboxy terminus to form a proline-extended biologically active peptide which is used to replace or add to at least a portion of at least one CDR region in an immunoglobulin molecule or fragment thereof. In another aspect, an immunoglobulin molecule or fragment thereof is provided which has either a TPO mimetic peptide or EPO mimetic peptide as a replacement for at least one native CDR region. In this aspect, the TPO mimetic peptide or EPO mimetic peptides may optionally be proline-extended as described herein.

In further particular embodiments the immunoglobulin molecule or fragment thereof is an Fab, a ScFv, a heavy chain variable region, a light chain or a full IgG molecule. The immunoglobulin molecule or fragment thereof can also have a dimerization domain, so as to enable immunoglobulin molecules which have only one CDR replaced with a peptide to dimerize and thus activate receptors that require dimerization for activation.

In certain embodiments, the biologically active peptide can be a linear peptide epitope or a discontinuous peptide epitope. Furthermore, the biologically active peptide, when substituted for a CDR region, can have in addition to proline, one, two or more additional flanking amino acid residues proximate to the amino and/or the carboxyl termini of the peptide, which are positioned between the peptide and immunoglobulin framework region residues (i.e., at what was the junction between a CDR and the adjoining framework). The flanking amino acid residues are not typically present in the active peptide. If preferred flanking amino acid residues are already known, the flanking amino acid residues are encoded by codons which designate those specific amino acid residues. However, by initially utilizing codons, such as NNK, NNY, NNR, NNS and the like, which designate multiple amino acid residues, a collection of peptides that differ from one another merely by the flanking residues is generated. The flanking amino acid residues may determine the presentation of the peptide in the immunoglobulin molecule or fragment thereof and thus may influence the binding and/or biological activity exhibited by the peptide. This random collection of flanking amino acids allows for the selection of the best context to display the peptide sequence within the antibody framework that results in specific binding to the target molecule and the exhibition of optimal biological activity. Screening of libraries of immunoglobulins having a common peptide but different flanking amino acid residues can be carried out using binding, growth and activation assays known by those skilled in the art and as described herein.

The peptide replacing the amino acid residues comprising a CDR can be any peptide which specifically binds a target molecule and whose utility could be altered by incorporation in an antibody framework. The peptide could also exhibit a specific activity (e.g., agonist, antagonist, enzymatic, etc.). In a particular embodiment the peptide is an agonist or an antagonist for a cell surface receptor. For example, the cell surface receptor can be for a cytokine, a growth factor, or a growth inhibitor.

In particularly useful embodiments, replacement of at least a portion of a CDR with a peptide provides an antibody that acts as an agonist. The peptide used to replace at least a portion of a CDR may itself have agonist properties. Alternatively, the peptide (although specifically binding to a receptor) may not exhibit agonist activity. Rather, agonist activity might be exhibited only when the peptide is substituted for at least a portion of a CDR and is thus present in the engineered antibody. In such embodiments, the presence or absence of proline flanking the peptide is not critical, but can, in some instances, be preferred.

Thus, in one aspect the present disclosure provides for an agonist antibody comprising an antibody framework engineered to contain at least one biologically active peptide inserted at, or in place of at least a portion of, one or more CDRs. The biologically active peptide may or may not exhibit agonist activity prior to insertion into the antibody framework. In certain embodiments the antibody framework is engineered to contain two peptides capable of dimerizing with each other.

In yet another aspect, the present disclosure provides for an immunoglobulin molecule or fragment thereof comprising a region where amino acid residues corresponding to at least a portion of a complementary determining region (CDR) are replaced with a biologically active peptide, whereby the immunoglobulin molecule or fragment thereof exhibits agonist activity. The biologically active peptide may or may not exhibit agonist activity prior to insertion into the antibody framework. In particularly useful embodiments the immunoglobulin molecule or fragment thereof exhibits c-mpl agonist activity.

In yet another aspect, the present disclosure provides for an immunoglobulin molecule or fragment thereof comprising a biologically active peptide inserted at a complementary determining region (CDR), whereby the immunoglobulin molecule or fragment thereof exhibits agonist activity.

In yet another aspect, the present disclosure provides for an immunoglobulin molecule or fragment thereof comprising a region where amino acid residues corresponding to at least a portion of a complementary determining region (CDR) are replaced with a biologically active peptide, whereby the immunoglobulin molecule or fragment thereof exhibits c-mpl agonist activity.

In further particular embodiments, the peptide replacing the amino acids of a CDR is an agonist TPO mimetic peptide. One such agonist peptide has at least the sequence IEGPTLRQWLAARA (SEQ. ID. NO. 1). Other sequences are possible for TPO agonist mimetic peptides, which can be found using binding, growth and activation assays known by those skilled in the art and as described herein. Agonist TPO mimetic peptides when positioned in CDR regions can have one or more additional amino acid residues at the amino and/or carboxyl termini of the peptide which become covalently bonded to immunoglobulin framework residues. One such TPO mimetic peptide has an additional proline residue added to the carboxyl terminus; IEGPTLRQWLAARAP (SEQ. ID. NO: 2). Other immunoglobulin molecules or fragments thereof have a CDR region replaced by the TPO mimetic peptides comprising the amino acid sequence of SEQ. ID. NOs: 25, 27, 29, 31, 33, 35, 37, 39, 41, 43, 45, 47, and 49 (see FIG. 5).

Another biologically active peptide that can replace the amino acid residues of a CDR is an agonist EPO mimetic peptide. One such EPO agonist peptide has as its amino acid sequence DYHCRMGPLTWVCKPLGG (SEQ. ID. NO: 3). Other amino acid sequences are possible for EPO agonist mimetic peptides, which can be found using binding, growth and activation assays known by those skilled in the art and as described herein. Agonist EPO mimetic peptides when located in CDR regions can also have one or more additional amino acid residues at the amino and/or carboxyl termini of the peptide which become covalently bonded to immunoglobulin residues. Thus, in particular embodiments provided herein are immunoglobulin molecules (IgG) or fragments (e.g., Fab, scFv, heavy or light chains) that have a CDR region replaced with a TPO or EPO mimetic peptide. For example, the TPO peptide can include at least the sequence IEGPTLRQWLAARA (SEQ. ID. NO:1) and may further optionally have an additional proline at the immediate downstream position. The EPO mimetic encompasses at least the sequence DYHCRMGPLTWVCKPLGG (SEQ. ID. NO: 3). Likewise, it may optionally have an additional proline at the immediate downstream position.

In further particular embodiments, the peptide replacing the amino acids of a CDR is a human brain natriuretic peptide (hBNP). One such peptide is hBNP-32 which has at least the sequence CFGRKMDRISSSSGLGC (SEQ. ID. NO. 172). Other amino acid sequences are possible for hBN mimetic peptides, which can be found using assays known by those skilled in the art and as described herein. When positioned in CDR regions, hBN peptides can have one or more additional amino acid residues at the amino and/or carboxyl termini of the peptide which become covalently bonded to immunoglobulin framework residues.

In further particular embodiments, the peptide replacing the amino acids of a CDR is a peptide involved in insulin production. Such peptides include exendin -4, GLP-1 (7-36), GPL-2 (1-34), glucagons and PACAP-38 which have at least the following sequences at least the sequences (SEQ. ID. NOs. 173-177).

Exendin-4:
HGEGRFTSDLSKQMEEEAVRLFIEWLKNGGPSSGAPPPS
GLP-1:
HAEGTFTSDVSSYLEGQAAKEFIAWLVKGR
GLP-2:
HADGSFSDEMNTILDNLAARDFINWLIQTKITDR
Glucagon:
HSQGTFTSDYSKYLDSRRAQDRVQWLMNT
PACAP-38:
HSDGIFTDSYSRYRKQMAVKKYLAAVLGKRYKQRVKNK

Other amino acid sequences are possible for mimetic peptides of exendin-4, GLP-1 (7-36), GPL-2 (1-34), glucagons or PACAP-38, which can be found using assays known by those skilled in the art and as described herein. When positioned in CDR regions, exendin4, GLP-1 (7-36), GPL-2 (1-34), glucagons or PACAP-38 peptides can have one or more additional amino acid residues at the amino and/or carboxyl termini of the peptide which become covalently bonded to immunoglobulin framework residues.

In further particular embodiments, the peptide replacing the amino acids of a CDR is an adipocyte-specific secretory protein. Examples of peptides that can be employed include functional portions of Adiponectin (Acrp30), the globular region of which has at least the sequence:

| | |
|---|---|
| FSVGLETYVTIPNMPIRFTKIFYNQQNHYDGS | (SEQ. ID. NO. 178) |
| TGKFHCNIPGLYYFAYHITVYMKDVKVSLFKK | |
| DKAMLFTYDQYQENNVDQASGSVLLHLEVGDQ | |
| VWLQVYGEGERNGLYADNDNDSTFTGFLLYHD | |
| TN. | |

Other amino acid sequences are possible for adipocyte-specific secretory mimetic peptides, which can be found using assays known by those skilled in the art and as described herein. When positioned in CDR regions, adipocyte-specific secretory peptides can have one or more additional amino acid residues at the amino and/or carboxyl termini of the peptide which become covalently bonded to immunoglobulin framework residues.

Any immunoglobulin molecule (antibody) or fragment thereof could potentially provide the framework and have a CDR replaced with a peptide according to the present disclosure. For therapeutic or in vivo diagnostic use it is preferable that the antibody is of human origin or humanized, such as an anti-tetanus toxoid immunoglobulin. Furthermore, independent of or in conjunction with the presence of additional flanking amino acids bound to the peptide, one or more amino acid residues in other regions of the immununoglobulin, other CDR region(s) and/or framework regions, can be altered to modify the binding, activity and/or expression displayed by the peptide in the context of the immunoglobulin molecule.

It is contemplated that after construction of biologically active recombinant antibodies and/or fragments thereof, such recombinants can be subjected to randomization methods known in the art to introduce mutations at one or more points in the sequence to alter the biological activity of the antibodies. After generation of such mutants using randomization methods such as those described herein, the resulting recombinants may be assayed for activity using binding, growth, expression and activation assays.

Further provided are nucleic acid molecules encoding immunoglobulin molecules or fragments thereof which have the amino acids of one or more CDR regions replaced by a biologically active peptide. These nucleic acid molecules can be present in an expression vector, which can be introduced (transfected) into a recombinant host cell for expression of these molecules. Also provided are methods of producing an immunoglobulin molecule or fragment thereof containing a biologically active peptide, comprising culturing a recombinant host cell under conditions such that the nucleic acid contained within the cell is expressed.

Also provided are compositions, comprising an immunoglobulin molecule or fragment thereof which has amino acid residues corresponding to a CDR replaced with amino acid residues comprising a TPO or EPO mimetic peptide and a pharmaceutically acceptable carrier.

Further provided are EPO mimetic peptides with additional flanking residues which are suitable for replacement of CDRs. Also provided are nucleic acid molecules encoding these peptides.

Further provided are methods of engineering immunoglobulin molecules or fragments thereof to exhibit an agonist activity in which a biologically active peptide replaces at least a portion of one or more CDR regions of light and/or heavy chains. The methods encompass inserting a nucleic acid molecule encoding a biologically active peptide in place of at least a CDR region of a nucleic acid molecule encoding an immunoglobulin heavy or light chain or adding the molecule to the native CDR sequence and then expressing the nucleic acid molecule encoding the immunoglobulin heavy or light chain variable domain along with its complementary variable region domain, such that the two domains associate.

Further provided are methods of engineering immunoglobulin molecules or fragments thereof to exhibit an activity (property) of a biologically active peptide in which a biologically active peptide replaces one or more CDR regions of light and/or heavy chains. The methods encompass inserting a nucleic acid molecule encoding a biologically active peptide in place of at least a portion of a CDR region of a nucleic acid molecule encoding an immunoglobulin heavy or light chain or adding the molecule to the native CDR sequence; and expressing the nucleic acid molecule encoding the immunoglobulin heavy or light chain variable domain along with its complementarity variable region domain, such that the two chains associate.

In another aspect, this disclosure provides a method for producing in a polypeptide a binding site capable of binding a preselected agent, the method including the steps of introducing a nucleotide sequence that codes for an amino acid residue sequence defining said binding site into a CDR region of a nucleic acid comprising an immunoglobulin heavy or light chain gene by amplifying the CDR region of the immunoglobulin gene, the introduced nucleotide sequence having the formula -Xₐ-Y-X_{b}wherein X is the same or different at each occurrence and represents a randomizing trinucleotide, the sum of a and b is 4 or less and Y is a nucleotide sequence that encodes a minimum recognition domain of said binding site. In particularly useful embodiments, amplification is achieved using overlap PCR, however, any known amplification technique could be employed, such as, for example, the methods disclosed in W094/18221, the disclosure of which is incorporated herein by reference.

In yet another aspect, this disclosure provides methods for creation of a library of monoclonal antibodies that can be screened for a desired activity. These methods of making a library include the steps of inserting a nucleic acid molecule encoding a biologically active peptide into, or in place of at least a portion of, one or more CDR regions of a nucleic acid molecule encoding an immunoglobulin heavy or light chain, providing up to a pair of randomizing trinucleotides on either side of the inserted nucleic acid molecule, and expressing a library of monoclonal antibodies. In particularly useful embodiments, a pair of randomizing trinucleotides is provided on both sides of the inserted nucleic acid molecules. The library of monoclonal antibodies thus produced can then be screened for a desired activity.

In a specific embodiment, antibodies and fragments thereof have different amino acids flanking the peptide at the amino and the carboxyl termini where the peptide becomes bound to the antibody scaffold. This, results in a population of antibody molecules or fragments thereof that may differ in the presentation of the peptide. The population is screened for those antibodies that exhibit the biological activity of the peptide. In a preferred embodiment, the amino acid immediately adjacent the peptide is a proline.

If the activity of the biologically active peptide is to activate a target molecule, this may require dimerization of two target molecules (e.g. receptors in the hematopoietic superfamilies). For dimerization to occur, two peptides must be positioned to each bind a target molecule such that the two bound target molecules can then properly associate. This can be accomplished by having two peptides present on the same antibody or fragment thereof or by causing two antibody molecules each containing one peptide to bind together. Thus, for example, a single peptide can be inserted into or substituted for at least a portion of a CDR and then expressed as an immunoglobulin or a F(ab′)₂ fragment. As another example, two peptides can be inserted into or substituted for at least a portion of one or more CDRs and expresses as any antibody or antibody fragment.

The screening of antibodies or fragments thereof can be accomplished by panning with cells that have surface molecules to which the peptide specifically binds. Solid phase binding using purified target molecules or fragments thereof can also be used. Binding can also be carried out in solution using labeled target molecules. In addition, antibodies or fragments thereof can be screened by the use of biological assays for agonist or antagonist activity of the peptide.

Also provided are libraries of different immunoglobulin molecules or fragments thereof wherein amino acid residues corresponding to a complementarity determining region (CDR) are replaced with amino acid residues comprising a biologically active peptide which has at least one additional amino acid residue at the amino or the carboxyl terminus and the immunoglobulin molecules or fragments thereof differ by the additional amino acid residue of the peptide.

In specific embodiments the biologically active peptide is a TPO mimetic or an EPO mimetic. The antibodies of the library are displayed on phage.

Further provided are methods of stimulating proliferation, differentiation or growth of cells, which include contacting the cells with an effective amount of an immunoglobulin molecule or fragment thereof having one or more CDRs replaced with a biologically active peptide which binds to a receptor on the cells surface. In specific embodiments the biologically active peptide is a TPO mimetic or an EPO mimetic.

In other specific embodiments, is provided a method of stimulating proliferation, differentiation or growth of megakaryocytes by contacting megakaryocytes with an effective amount of an immunoglobulin molecule or fragment thereof having one or more CDRs replaced with a TPO mimetic peptide. Also provided is a method of increasing platelet production, which involves contacting megakaryocytes with an effective amount of an immunoglobulin molecule or fragment thereof having one or more CDR regions replaced with a TPO mimetic peptide. Also provided is a method of stimulating megakaryocytes and/or increasing platelet production in a patient, in which an effective amount of an immunoglobulin molecule or fragment thereof having one or more CDRs replaced with a TPO mimetic peptide is administered to a patient in need thereof. The immunoglobulin molecule and the megagakarocytes can also be contacted in vitro and the resultant cells can be introduced into the patient. In addition, an antibody or fragment thereof having at least one TPO mimetic peptide incorporated therein can be administered to a subject who intends to donate platelets, thus increasing the capacity of a donor to generate platelets to provide a more robust source of such platelets.

Also provided herein is a method of stimulating proliferation, differentiation or growth of hematopoietic cells, comprising contacting the cells with an effective amount of an immunoglobulin molecule or fragment thereof having one or more CDRs replaced with a EPO mimetic peptide.

Further provided are methods of treating congestive heart failure (CHF), which include administering an effective amount of an immunoglobulin molecule or fragment thereof having one or more CDRs replaced with a biologically active peptide which exerts diuresis, natriuresis, or vasodilatation. In specific embodiments the biologically active peptide is a hBNP or a hBNP mimetic.

Also provided are methods of diabetes, which include adminstering an effective amount of an immunoglobulin molecule or fragment thereof having one or more CDRs replaced with a biologically active peptide. In particularly useful embodiments the immunoglobulin molecule or fragment thereof exhibits NPR-A, GLP-1 receptor, AdipoR1, or AdipoR2 agonist activity. In specific embodiments the biologically active peptide is exendin-4, GLP-1 (7-36), GPL-2 (1-34), glucagons or PACAP-38 peptide or a peptide mimetic of one of the foregoing peptides.

Also embodied herein is a method of activating a homodimeric receptor protein, by contacting the receptor with an immunoglobulin molecule or fragment thereof having a CDR region replaced with a biologically active peptide that specifically binds the receptor and which has been dimerized. In a further embodiment the receptor is a thrombopoietin receptor.

### DESCRIPTION OF THE DRAWINGS

FIG. 1 is a diagrammatic representation of the vector pRL4.

FIGS. 2A and B show the sequence of the human tetanus toxoid antibody framework, light and heavy chains, respectively.

FIG. 3 is a diagram depicting the grafting of the TPO mimetic peptide AF12505 into the heavy chain CDR3 region of the tetanus toxoid framework antibody. XX represents flanking random amino acids.

FIG. 4 is a diagram of the construction of a peptide cloned into the heavy chain CDR3 region.

FIG. 5 represents the amino acid and nucleotide sequences of clones that encode TPO mimetic peptide AF1205 with different random flanking residues.

FIG. 6A-C depicts the nucleic acid sequence of plasmid pRL8 (SEQ. ID. NO: 60). pRL8 is a modified version of pRL4 (pRL4 is also known as pComb 3X). The pRL4 was modified between the Spe I and neighboring Sfi I restriction sites (shown by underlining) to include a flexible linker (murine kappa hinge region) followed by a Jun leucine zipper dimerization domain.

FIG. 7 is a schematic depiction of a portion of the plasmid pRL8.

FIG. 8 depicts the nucleic acid sequence of a portion of plasmid pRL8 (SEQ. ID. NO: 52) along with amino acid sequences corresponding to certain delineated nucleic acid sequences (SEQ. ID. NO: 53).

FIG. 9 is a chart showing sequences of certain TPO positive clones herein.

FIG. 10 is a bar graph showing activity of certain Fab clones containing 2 TPO mimetic peptides.

FIG. 11 is a bar graph showing activity of certain Fab clones containing 2 or 3 TPO mimetic peptides.

FIG. 12 graphically depicts the activity of Clone 59 as reflected by induction of luciferase activity.

FIG. 13A depicts the amino acid sequence and nucleic acid sequence of the 5G1.1-TPO heavy chain (SEQ. ID. NOS: 67 and 68, respectively).

FIG. 13B depicts the amino acid sequence and nucleic acid sequence of the 5G1.1 light chain (SEQ. ID. NOS: 69 and 70, respectively).

FIG. 14 is a bar graph showing FACS analysis of cMpl receptor binding of purified 5G1.1+TPO mimetic peptide compared to parental 5G1.1 antibody.

FIG. 15 is a bar graph showing comparative activity of 5G1.1 antibody containing the TPO mimetic peptide in connection with cells transfected with a control vector containing no cMpl-R and cells transfected with a vector containing cMpl-R.

FIG. 16 shows the sequence of clone 429/Xb4 (SEQ. ID. NO: 116)

FIG. 17 is a flow chart showing the initial steps for making vector pRL5-Kappa.

FIG. 18 is a flow chart showing additional steps for making vector pRL5-Kappa.

FIG. 19 is a map of vector pRL5.

FIG. 20 is a schematic of vector pRL5-Kappa.

FIG. 21A-I show the nucleic acid sequence of vector pRL5-Kappa.

FIGS. 22 and 23 show the human germlne sequences with the highest homology to the TT-TPO starting antibody.

FIGS. 24 and 25 show the nucleic acid and amino acid sequences of the pAXB116Fab′ heavy and light chain variable regions, respectively.

FIG. 26 shows the nucleic acid sequences of the primers used to generate the pAXB116 heavy chain.

FIG. 27 shows the nucleic acid sequences of the primers used to generate the pAXB116 light chain.

FIG. 28 schematically shows the construction scheme for the pING-pAXB116 vector.

FIG. 29 shows the amino acid sequences for the heavy and light chain of clone 116.

FIG. 30 shows the result of SDS-PAGE of pAXB116.

FIG. 31 shows the proliferative effect of TPO and pAXB116 on CD34+cord blood cells.

FIG. 32 shows the activity of clone 116.

FIG. 33 shows the sequences of heavy chain clones in accordance with an alternative embodiment of the present disclosure.

FIG. 34 shows the relative activity of various H2/H3-(X4b) clones in 6 cm luciferase assays.

FIG. 35 shows the effect of the addition of 3 original TT amino acids on the placement of the TPO peptide in the HC-CDR2.

FIGS. 36A-E show the nucleic acid sequence (SEQ. ID NO. 141) of clone pRL5-116F and the amino acid sequences of the 116 light chain (SEQ. ID NO. 142) and the 116 heavy chain (SEQ. ID NO. 143).

FIG. 37 shows the relative activity of 116 mutants in 6 cm luciferase assays.

FIG. 38 shows the amino acid sequences of various 116 variant clones.

### DETAILED DESCRIPTION

As used herein, “immunoglobulin” refers to an entire immunoglobulin molecule or molecules that contain immunologically active portions of whole immunoglobulin molecules and includes Fab, F(ab [symbol] )2, scFv, Fv, heavy chain variable regions and light chain variable regions. The terms immunoglobulin and antibody are used interchangeably herein.

Any peptide that exhibits a useful property is suitable for insertion in an antibody framework. Peptide activities and uses include, but are not limited to, binding a receptor, binding a membrane bound surface molecule, binding a ligand, binding an enzyme or structural protein, activating or inhibiting a receptor, targeted drug delivery, or any enzymatic activity. Those peptides whose utility can be increased from the enhanced stability conferred upon them when presented in the context of an immunoglobulin molecule are usually selected. It should be understood that “biological activity” as used herein includes any activity associated with a molecule having activity in a biological system, including, but not limited to, the stimulatory or inhibitory activity triggered by protein-protein interactions as well as the kinetics surrounding such interactions including the stability of a protein-porotein complex. Enhancing or increasing “biological activity” herein is meant to include an increase in overall activity or an increase in any component of overall activity. It should be understood that a peptide may exhibit one biological activity (such as, e.g., simply binding to a target) before insertion into the antibody framework, and a different or enhanced biological activity (such as, e.g., agonist activity) after insertion into the antibody framework.

Many peptides which could benefit from display in the context of an immunoglobulin have been identified and are known to those who practice the art, e.g., EPO and TPO mimetic peptides. Other examples include peptides that bind to receptors which are activated by ligand-induced homo-dimerization including active fragments displaying G-CSF activity, GHR activity and prolactin activity as described in Whitty and Borysenko, Chem Biol., (1999) Apr 6(4):R107-18; other examples of suitable peptides include a nerve growth factor mimetic from the CD loop as described in Zaccaro et al., Med. Chem. (2000) 43(19); 353040; an IL-2 mimetic as described in Eckenberg, et al., J. Immunol. (2000) 165(8):4312-8; glucogon-like peptide-1 as described in Evans et al., Drugs R.D. (1999) 2(2): 75-94; tetrapeptide I (D-lysine-L-asparaginyl-L-prolyl-L-tyrosine) which stimulates mitogen activated B cell proliferation as described in Gagnon et al., Vaccine (2000) 18(18):1886-92. Peptides which exhibit receptor antagonistic activity are also contemplated. For example, N-terminal peptide of vMIP-II as an antagonist of CXCR4 for HIV therapy as described in Luo et al., Biochemistry (2000) 39(44): 13545-50; antagonist peptide ligand (AFLARAA) of the thrombin receptor for antithrombotic therapy as described in Pakala et al., Thromb. Res. (2000) 100(1): 89-96; peptide CGRP receptor antagonist CGRP (8-37) for attenuating tolerance to narcotics as described in Powell et al., Br. J. Pharmacol. (2000) 131(5): 875-84; parathyroid hormone (PTH)-1 receptor antagonist known as tuberoinfundibular peptide (7-39) as described in Hoare et al., J. Pharmacol. Exp. Ther. (2000) 295(2):761-70; opioid growth factor as described in Zagon et al., Int. J. Oncol. (2000) 17(5): 1053-61; high affinity type I interleukin 1 receptor antagonists as disclosed in Yanofsky, et al., Proc. Natl. Acad. Sci. USA, Vol. 93, pp. 7381-7386, July 1996 and Vigers, et al., J. Biol. Chem., Vol 275, No 47, pages 36927-36933, 2000; and acid fibroblast growth factor binding peptide as described in Fan et al., IUBMB Life (2000) 49 (6) 545-48. Further examples of biologically active peptide which can be incorporated into antibodies or antibody fragments in accordance with this disclosure include proteins secreted by the heart as part of the body's response to congestive heart failure, such as, for example, human brain natriuretic peptide (hBNP) as described in Mukoyama, et al., J. Clin. Invest. 87(4): 1402-12 (1991) and Clemens, et al., J. Pharnacol. Exp. Ther. 287(1): 67-71(1998). Additional examples of biologically active peptide which can be used in accordance with this disclosure include proteins which have the potential to preserve or improve beta-cell function (e.g., by inducing glucose-dependent insulinotropic effect), such as, for example, exendin4, GLP-1 (7-36), GPL-2 (1-34), glucagons or PACAP-38 (see, Raufman, et al., J. Biol. Chem. 267(30): 21432-7 (1992).)

Peptides can also be discovered using methods familiar to those skilled in the art. In order to identify a region of a protein that is involved in a specific biological function, a survey of the shorter peptide fragments making up that protein may reveal the linear peptide epitope responsible. Alternatively by surveying libraries of random peptides, a peptide that represents an optimal linear epitope or a discontinuous epitope may be discovered that mimics the activity of the natural protein. One method for selection is termed peptide phage-display. In this approach, a random peptide epitope library is generated so that peptides are present on the surface of a bacteriophage particle. These collections, or libraries, of peptides can then be surveyed for those able to bind to a specific immobilized target protein. (Pasqualini, R. et al., j. Cell Biol., 130, 1995, 1189-1196; Wrighton, N. C., et al., Science, 273, 1996, pages 458-463; Cwirla, S. E., et al., Science, 276, 1997, pages 1696-1699; Koivunen et al, J. Biol, Chem., 268, 1993, pages 20205-20210; Koivunen et al., Bio/Technol., 13, 1995, pages 265-270; Healy et al., Biochem., 34, 1995, pages 3948-3955; Pasqualini et al., J. Cell Biol., 130, 1995, pages 1189-1196). Alternative peptide selection systems are also possible including cell surface display and ribosomal display.

Peptide mimetics used in accordance with this description are generally less than or equal to the number of amino acid residues that make up a CDR region, although they could be longer.

Any antibody can serve as a scaffold sequence, however typically human antibodies are chosen as human therapeutics is one of the ultimate objectives. Human or humanized antibodies are less likely to cause an adverse immune response in a human patient. The major criteria in selecting an antibody to serve as a framework for insertion of a peptide, is that the replacement of one or more CDRs of the antibody with the peptide must change the antigen specificity. The antibody can be a complete antibody or an Fab, scFv or F(ab′)₂ fragment or portion thereof.

Alternatively, a library of antibodies can have one or more heavy and/or light chain CDRs replaced with a desired peptide. The resulting library can then be screened to identify antibodies having a desired activity. It should be understood that randomization with in the substituted peptide can also be provided to generate an antibody library.

A useful antibody is the anti-tetanus toxoid (TT) Fab, as it is human and because modification of the HCDR3 is sufficient to change the antigen specificity of the antibody (Barbas et al., J. Am. Chem. Soc., 116, 1994, pages 2161-2162 and Barbas et al., Proc. Natl. Acad. Sci. USA, 92, 1995, pages 2529-2533).

Grafting of the DNA sequence of the peptide of choice into an antibody so as to replace the CDR(s) of an antibody with the peptide sequence is carried out using recombinant DNA techniques known to those skilled in the art.

Examples of methods which can be utilized to graft a desired peptide having biological activity in place of a CDR region include, but are not limited to, PCR overlap, restriction enzyme site cloning, site specific mutagenesis and completely synthetic means. For a description of techniques involving overlap PCR, see, e.g., Example 1 herein. Site specific mutagenesis can be accomplished in several ways. One is based on dut/ung Kunkel mutagenesis (Kunkel, T. A., Proc. Natl. Acad. Sci. (1985) vol. 82, pp.488-92). The Muta-Gene in Vitro Mutagenesis kit is available from BioRad based on this methodology (cat. # 170-3581 or 170-3580). Several PCR amplification based mutagenesis approaches are also commercially available such as Stratagene's QuickChange Site-Directed Mutagenesis Kit and the ExSite PCR-based Site-Directed Mutagenesis Kit. Another non-PCR method is available from Promega as the GeneEditor in vitro Site-Directed Mutagenesis System. Completely synthetic means are also well-known and described, e.g., in Deng, et al., Methods Mol. Biol. (1995) 51 :32942; Kutemeler et al., Biotechniques, (1994) 17(2): 242-246; Shi et al., PCR Methods Appl., (1993) 3(1): 46-53 and Knuppik et al., J. Mol. Biol., (2000) 11:296(1): 571-86 each incorporated herein by reference. In addition, the above methods used for replacing all or a portion of at least one CDR sequence can be utilized to graft a desired peptide into or adjacent to at least one native CDR sequence without replacing the original CDR sequence. In this manner, a CDR/biologically active peptide mimetic fusion construct is formed.

It is contemplated that flanking sequences may be added to the carboxyl and/or amino terminal ends of the biologically active peptide. Flanking sequences can be useful to reduce structural constraints on the grafted peptide to allow it to more easily adopt a conformation necessary for biological activity. In a preferred embodiment, a flanking region including a proline is covalently attached to the carboxy terminus of the biologically active peptide to create a proline extended biologically active peptide.

In one embodiment, a flanking region can be generated by randomizing two amino acid positions on each side of the peptide graft in order to determine the best sequence. In this manner, a library having members with multiple varied sequences can be generated. The resulting constructs are then tested for biological activity as described below by, e.g., panning techniques. Recombinant proteins can be generated that have random amino acids at specific positions. This can be accomplished by modifying the encoding DNA. When introducing randomization at a specific amino acid's codon position, a preferable deoxyribonucleotide “doping strategy” is (NNK)ₓ in order to cover all 20 amino acids and to minimize the number of encoded stop codons. Accordingly, N may be A, C, G, or T (nominally equimolar), K is G or T (nominally equimolar), and x is typically up to about 5, 6, 7, or 8 or more, thereby producing libraries of mono-, di-, tri-, quadra-, penta-, hexa-, hepta-, and octa-peptides or more. The third position may also be G or C, designated “S”. Thus, NNK or NNS (i) code for all the amino acids, (ii) code for only one stop codon, and (iii) reduce the range of codon bias from 6:1 to 3:1. There are 32 possible codons resulting from the NNK motif: 1 for each of 12 amino acids, 2 for each of 5 amino acids, 3 for each of 3 amino acids, and only one of the three stop codons. Other alternatives include, but are not limited to:

(NNN)ₓ which would provide all possible amino acids and all stops;

(NNY)ₓ eliminates all stops and still cover 14 of 20 amino acids;

(NNR)ₓ covers 14 of 20 amino acids; and

(NNS)ₓ covers all 20 amino acids and only one stop.

The third nucleotide position in the codon can be custom engineered using any of the known degenerate mixtures. However, the group NNK, NNN, NNY, NNR, NNS cover the most commonly used doping strategies and the ones used herein.

The collection of engineered antibodies that are created during this process can be surveyed for those that exhibit properties of the peptide as, e.g., phage displayed antibodies, essentially as has been described in Barbas, C. F., III, Kang, A. S., Lerner R. A., and Benkovic, S. J., Assembly of combinatorial antibody libraries on phage surfaces: the gene III site, Proc. Natl. Acad. Sci. USA, 88,1991, pages 7978-7982 incorporated herein by reference. This technology allows recombinant antibodies (as complete antibodies, Fab F(ab′)₂, or scFv) to be expressed on the surface of a filamentous bacteriophage. That same phage will have within it the genes encoding that specific antibody.

It is contemplated that any other known method of introducing randomization into a sequence may be utilized herein. For example, error prone PCR can introduce random mutations into nucleic acid sequences (See, e.g., Hawkins et al., J. Mol. Biol, (1992) 226(3): 889-96). Briefly, PCR is run under conditions which compromise the fidelity of replication, thus introducing random mutations in sequences as those skilled in the art would accomplish. After generation of such random mutants, they can be placed into phage display formats, panned and thus evaluated for activity. Likewise, particular bacteria known to provide random mutations of genes, such as Epicurian Coli® XL1-Red Competent cells (commercially available from Stratagen, La Jolla, Calif.), which do so during plasmid replication can be utilized to provide random mutants which are then screened for biological activity in accordance with the present disclosure.

It is also contemplated that randomization may be introduced at any point in the nucleotide sequence after incorporation of an active peptide into the antibody or fragment thereof to alter the overall biological activity of the antibody. In this manner, not only can alterations be made in the biological activity of a peptide mimetic by causing mutations within the peptide's sequence, but mutations in the surrounding scaffold can be incorporated with the resulting constructs being assayed for alterations in biological activity or expression. Indeed, it is contemplated that libraries having repertoires of multiple constructs resulting from such randomization can be generated and assayed.

Single chain libraries can be utilized in accordance with the present disclosure because an entire binding domain is contained on one polypeptide. The light chain variable region is separated from heavy chain variable region by a linker region. The use of short linkers (<11 amino acids) favors a dimeric complex where V_{H} of one ScFv associates with V_{L} of another ScFv molecule and visa versa, these molecules are termed diabodies (Kortt, A. A., Malky, R. L., Caldwell, J. B., Gruen, L. C., Ivanci, N., Lawrence, M. G. et al. Eur. J. Biochem. 221:151-157, 1994). This is because folding of monomeric ScFv is impaired with linkers <11 amino acids (Alfthan, K., Takkinen, K., Sizman, D., Soderlund, H., and Teeri, T. T. Protein-Eng. 8:725-731, 1995). Longer linkers (>11 amino acids) favors folding of monomeric ScFv into a single antigen binding domain, thus precluding dimer formation.

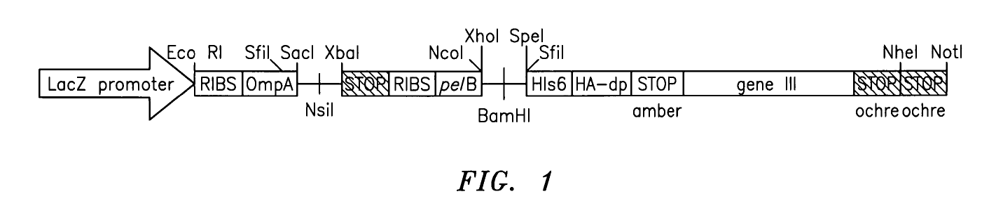

One useful phage display vector is pRL4 which is also known as pComb 3X (see FIG. 1). This vector enables display of chimeric expression products on the surface of packaged phagemid particles. pRL4 is a modified version of pComb3H (Barbas, C. F. III and Burton, D. R. 1994. Monoclonal Antibodies from Combinatorial Libraries. Cold Spring Harbor Laboratory Course Manual, Cold Spring Harbor, N.Y.; Burton, D. R.; Barbas, C. F. III. Advances in Immunology 57:191-280, 1994; Lang, I. M., Chuang, T. L., Barbas, C. F. 3^{rd}, Schleef, R. R. J. Biol. Chem. 271: 30126-30135,1996; Rader and Barbas, Phage Display, A Laboratory Manual, Cold Spring Harbor Laboratory Press, Cold Spring Harbor, N.Y. (2000). The design of pRL4 allows for dimerization of scFv antigen binding domains on the phage surface and in soluble form as detailed below. When the plasmid is transformed into a supE bacterial host such as ER2537 (F′ Sup E, New England Biolabs, Beverly, Mass.), the amber mutation is suppressed approximately fifty percent of the time. In this way half of the expressed scFvs are fused with the filamentous phage gene III protein (amino acids 230-406) and the other half will be terminated just prior to gene III to produce soluble scFv. Both the scFv-pIII fusion and soluble scFv products have the Omp A signal sequence and will be transported to the periplasm where they will be able to form dimeric scFv complexes, termed diabodies (Kortt, A. A., Malby, R. L., Caldwell, J. B., Gruen, L. C., ivanci, N., Lawrence, M. C. et al. Eur. J. Biochem. 221: 151-157, 1994). Diabodies are expected to fold such that the V_{H} of one scFv will pair with the V_{L} of a second scFv-pIII resulting in divalent antibody fragments. In a non-sup E host, such as TOP1OF′ (InVitrogen, Carlsbad, Calif.), the amber stop codon is recognized yielding soluble scFv diabodies.

In the final single chain expression construct in pRL4, the single chain antibody fragments are cloned downstream of the E. coli lacZ promoter, ribosome binding site, and omp A leader sequence. These elements allow induction of expression by IPTG, and the secretion out of the cell via the omp A leader sequence when expressed in the suppressor strain ER2537. The single chain fragments are fused in frame with filamentous phage gene III (gIII) sequences (amino acids 230-406). The gIII protein product, pIII, is a minor coat protein necessary for infectivity. Upon promoter induction by IPTG, the single chain antibody-pIII fusion is synthesized and transported to the bacterial periplasmic space. In the periplasmic space, the scFv-gene III fusion proteins are inserted into the membrane. Upon superinfection with helper phage, these fragments are exported out of the cell on the surface of phage as pIII-antibody fragments. Other possible proteins to be used for fusion on the surface of phagemids include filamentous coat protein pVIII and other coat proteins.

Fab fragment libraries, that maintain the native antigen recognition site, are useful to ensure that affinity is maintained.

In the final hybrid Fab expression construct in pRL4, the light and heavy chains are cloned as a single Sfil fragment. In this way, the light chain fragments are cloned downstream of the E. coli lacZ promoter, ribosome binding site, and omp A leader sequence. These elements allow induction of expression by IPTG, and secretion out of the cell via the omp A leader sequence. The light chain fragments are followed by a stop codon, a second ribosome binding site, the E. coli pel B leader sequence and heavy chain. Hybrid heavy chain genes are fused in frame with filamentous phage gene III (gIII) sequences (amino acids 230-406). An amber stop codon is present at the fusion junction. In a sup E bacterial host such as ER2357 (New England Biolabs, Beverly, Mass.), the amber mutation is suppressed. Upon promoter induction, a single polycistronic message is transcribed and translated as two polypeptides, a light chain and a heavy chain-gene III fusion protein. Following synthesis the polypeptides are transported to the bacterial periplasmic space as directed by the leader sequences. In the periplasmic space the heavy chain-pIII fusion proteins are inserted into the membrane, and the light and heavy chains are associated covalently through disulfide bonds, forming the antigen binding sites. The human constant region CH1 and C_{L} sequences include the cysteines that form the disulfide bond between heavy and light chains. Upon superinfection with helper phage, these fragments are exported out of the cell on the surface of phage as Fab-cpIII fusion. In a non-sup E host, such as TOP10F′ (Invitrogen, Carlsbad, Calif.), the amber stop codon is recognized yielding soluble Fab fragments. Important features of the pRL4 phage display system used include a purification His 6 tag, an HA epitope tag following the heavy chain, as well as a suppressible amber stop codon which is located between the heavy chain and the phage gene III. The HA tag is recognized by HA.11 antibody (Babco, Berkeley, Calif.) and 12CA5 antibody (Roche Molecular Biochemical, Indianapolis, Ind.). The His6 tag allows affinity purification of antibody fragments by Nickel-chelate chromatograph (Qiagen, Valencia, Calif.). The amber stop allows for quick conversion from a fusion Fab-cpIII product (for incorporation on the phage coat) when the stop is suppressed, to the soluble Fab which is made in a non-suppressor bacterial host.

Selection involves isolating from the library the best candidates that specifically bind to the peptides target molecule and display biological activity.

The phage expressing antibody fragments on their surface can be produced and concentrated so that all members of a library can be allowed to bind to the target molecule. The target molecule can be immobilized on a microtiter dish, on whole cells, the membranes of whole cells, or present in solution. Non-specific Ab-phage are washed away, and bound phage particles are released from the antigen, often by the use of low pH. The recovered Ab-phage are infectious and so can be amplified in a bacterial host. Typically, multiple rounds of this sort of selection are performed. Individual antibody fragment clones can then be analyzed as soluble Fabs or scFvs for identification of those that specifically recognize the target molecule.

Prior to any selection strategy, initial libraries are electroporated into host cells, such as ER2537. Library cultures are grown to log phase and superinfected with helper phage, such as VCSM13, a commercially available helper phage (Stratagene, La Jolla, Calif.). Superinfection provides the remaining phage components needed for packaging plasmids into phagemid particles. Alternatively, phage display without the use of helper phage may be utilized. Following overnight growth, phagemids in the culture supernate are precipitated with polyethylene glycol (PEG). PEG precipitated phage are used in panning (solid phase cell surface, internalization and membrane), FACS sorting, or magnetic sorting to purify specific binding antibodies from non specific binders.

In cell based panning, antibody-phage libraries are incubated with target cells, and the non-adherent phage are removed with multiple washes. A typical panning protocol is as follows:

1. Block phage particles with PBS+1 % BSA or 10% FBS+4% milk powder+NaN₃, (except when internalized antibodies are assayed).

2. Add target cells to blocked phages (approximately 5×10⁶ cells).

3. Mix and rotate slowly at 40° C. or 37° C.

4. Wash cells twice with 1 ml ice cold PBS/1 %BSA/NaN₃ or room temperature PBS/1%BSA/NaN₃.

5. Specific antibody-phage bound to cells can be eluted by low pH, for example with 76 mM citric acid ph 2.5 in PBS for 5 to 10 minutes at room temperature.

6. Neutralize eluted phage with 1M Tris-HCI pH 7.4.

7. After neutralization, antibody-phage can be used to infect ER2537 bacteria and amplify during overnight growth for the next round of panning.

Generally, 3-4rounds of panning are performed on each library. Phage ELISAs using commercially available secondary antibody (sheep anti-M13 antibody-HRP) or soluble antibody ELISAs using a commercially available HA. 11 antibody (Babco, Berkeley, Calif.) that recognizes the HA tag incorporated into each antibody from PRL4 sequences, can be performed following each round of panning to allow estimation of the enrichment of binding antibodies over non-binders. Following the last round of panning, the antibody-phage can be picked as single colonies from agar plates, grown as monoclonal antibody-phage and screened by ELISA for identification of specific binders. FACS analysis may also be utilized. Specifically the antibody-phage are infected into Top10F′ bacteria and plated for single colonies. Single colonies are picked form agar plates, grown and induced with IPTG. Soluble antibody is screened by ELISA for identification of specific binders. Screening can be done against live cells, against intact, mildly fixed target cells, or recombinant protein(s).

Methods for whole cell panning have been described previously (Siegel, D. L., Chang, T. Y., Russell, S. L., and Bunya, V. Y. 1997. J. Immunol. Methods 206:73-85 incorporated herein by reference). Other techniques for selection which can be applied include fluorescent activated cell sorting (FACs). Alternative methods for selection using libraries include, but are not limited to, ribosome display and plaque hybridization to a labeled antigen.

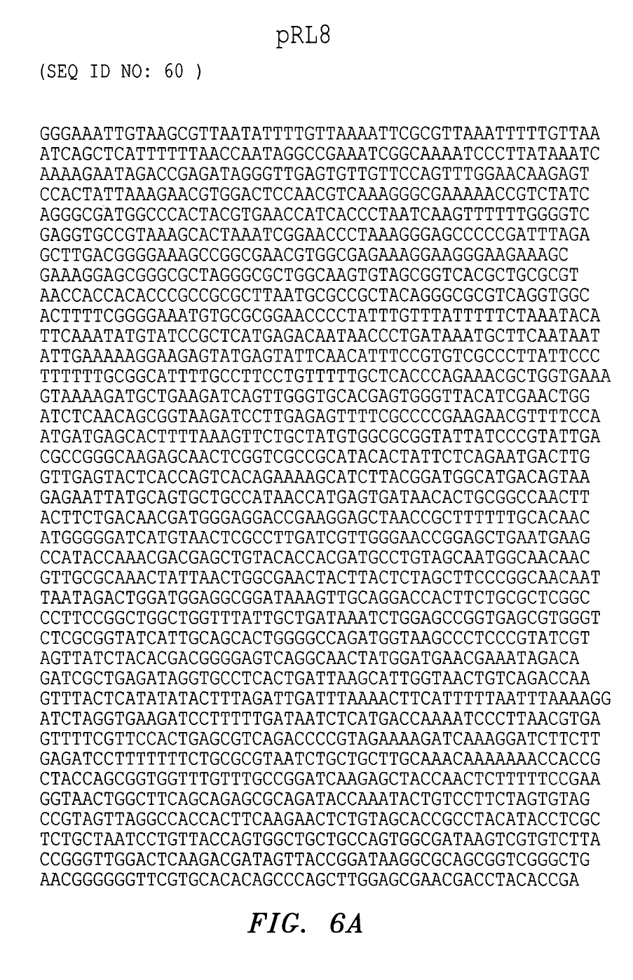

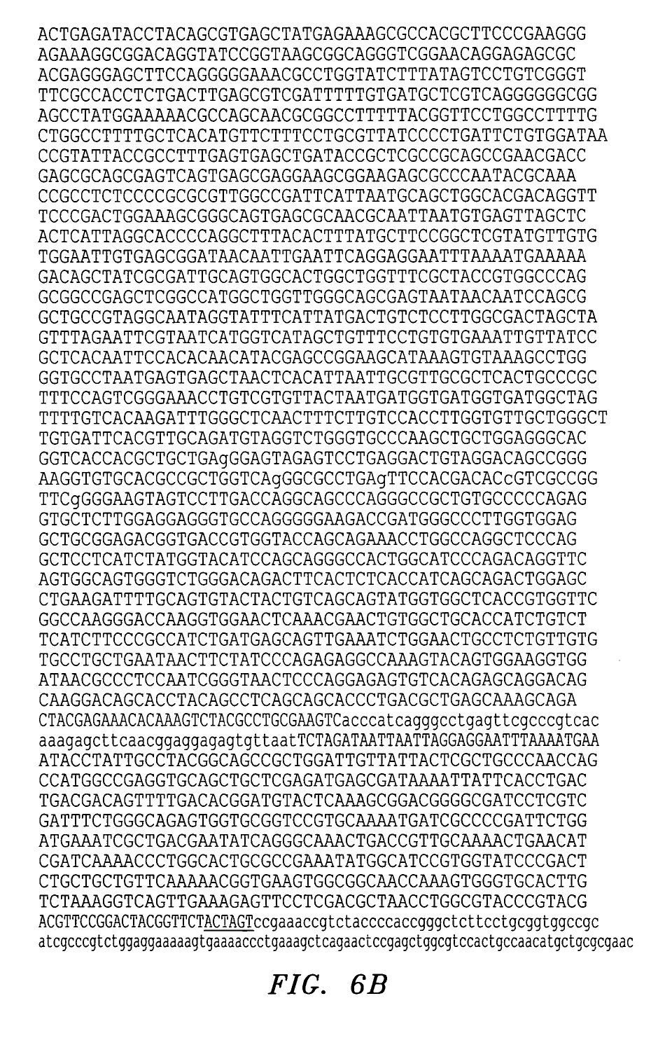

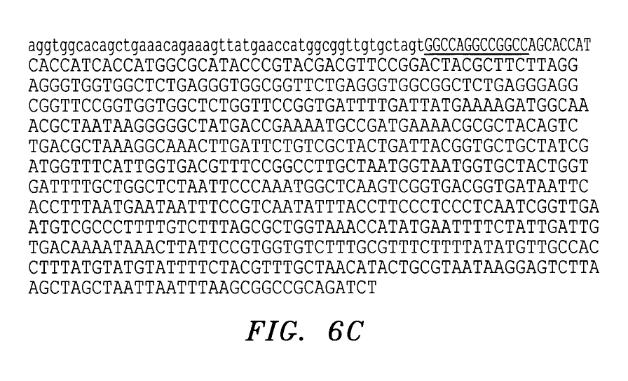

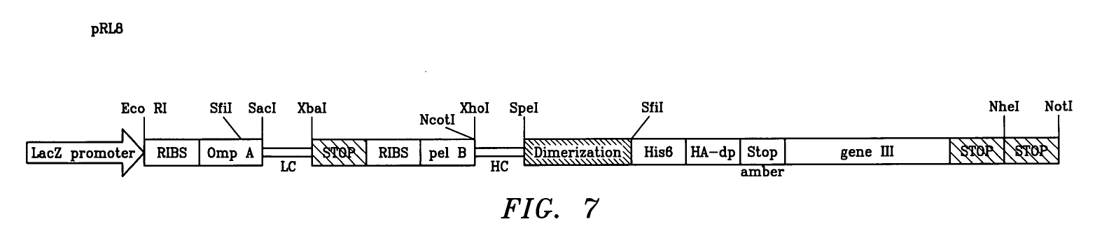

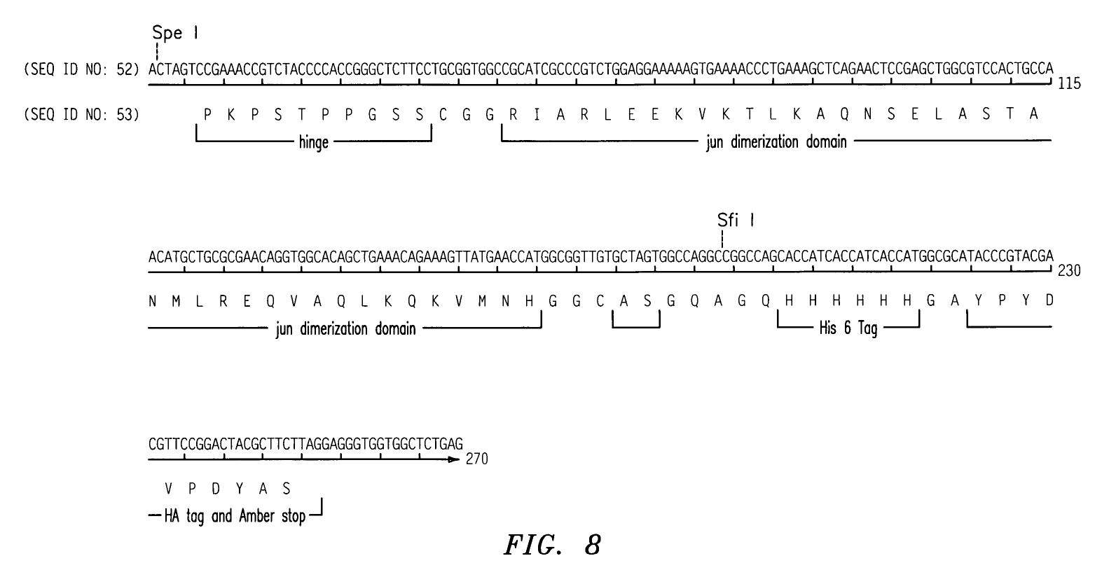

Following panning to isolate high affinity antibody binders, bioassays for functional screens of agonist antibodies can be carried out. Dimerization is often a prerequisite for activation of many receptors and thus bioassays focus on agonist antibodies that stimulate receptors via promotion of dimerization. As previously described, single chain multivalency is approached in linker design. Fab fragment multivalency can be approached in a number of ways. A number of recent reports in the literature have shown success in dimeric antibody fragment formation which is applicable to phage display (DeKruif, J., and Logtenberg, T. 1996. J. Biol. Chem. 271:7630-7634, Pack, P., and Pluckthun, A. 1992. Biochemistry 31:1579-1584, and Holliger, P., and Winter, G. 1993. Current Opin. Biotech. 4:446-449). Divalent Fabs can be created in at least two ways. In one approach dimerization is achieved by addition of a dimerization domain to pRL4, forming pRL8 (See FIGS. 6A-C, 7 and 8). There are a number of dimerization domains (lexA, Zn fingers, fos, jun etc.) that can be utilized in these vectors to obtain multivalency of Fab fragments. Dimerization domains are selected from, but not limited to, the following: jun (DeKruif, J. and Logtenberg, T. J. Biol. Chem. 271:7630-7634,1996; Kostelny, S. A., Cole, M. S., and Tso, J. Y. J.Immunol. 148:1547-1553,1992) the LexA dimerization region (Kim, B. and Little, J. W. Science 255:203-206,1992), the yeast GCN4 dimerization domain (van Heeckeren, W. J., Sellers, J. W., Struhl, K. Nucleic Acids Res. 20:3721-3724,1992), Gin invertase from the bacteriophage Mu (Spaeny-Dekking, L., Schlicher, E., Franken, K., van de Putte, P., Goosen, N. J. Bacteriol. 34:1779-1786,1995), E. coli NTRC protein dimerization domain (Klose, K. E., North, A. K., Stedman, K. M., Kustu, S. J. Mol. Biol. 241:233-245,1994), and HSV-1 ICP4 dimerization domain (Gallinari, P., Wiebauer, K., Nardi, M. C., Jiricny, J. J. Virol. 68:3809-3820,1994) all incorporated by reference. Also, a high temperature dimer domain from thermus organisms can be utilized (MacBeath, G., Kast, P., Hilvert, D., Biochemistry 37:100062-73, 1998 and MacBeath, G., Kast, P., Hilvert, D., Science 279:1958-61,1998). These are functional domains that when incorporated into a molecule allow for dimerization to occur. In addition, dimerization can be achieved in cells through the use of full lgG vectors, or dimerization domains such as CH3 dimerization domains. Those of ordinary skill in the art are familiar with these and other dimerization domains and their use to dimerize proteins.

Additional methods that may be utilized to generate antibody constructs which contain at least two binding sites are known. The antibody or antibody fragments created by each of these approaches could be utilized for testing agonistic antibody activity as described in Example 1 below for whole IgG produced in mammalian cells. These methods include chemical dimerization of Fab, pegylation of Fab, production of Fab′2, generation of whole IgG in bacterial cells, and use of diabodies (scFvs). Importantly, any of the antibody forms generated for analysis of agonistic activity could be used as the final therapeutic product.

Chemical dimerization may be also achieved using a variety of chemical crosslinking reagents. For example, SMCC (Succinimidyl trans-4 (maleimidylmethyl) cyclohexane-1-carboxylate) from Molecular Probes (Eugene, Ore.), Cat #S-1534. This reagent will modify primary amino groups in the antibody. After incubating the antibody with the SMCC at room temperature, the reaction is run over a PD-10 column. This maleimide derivitized Fab can be added to either a second Fab or a separate batch of the same Fab that has been treated with TCEP [(Tris (2-carboxyethyl) phosphine, hydrochloride): Molecular Probes Cat #T-2556] to reduce the thiol groups to SH. The reduction reaction is carried out in the dark for 15 minutes. The conjugation of the maleimide Fab and the thiol reduced Fab occurs at a 1:1 ratio. Dimers are isolated by passing the reaction over a sephadex 200 gel filtration column. Other chemical linkers known to those skilled in the art may be used for dimerization. Production of an Fab′ that has an extra cysteine residue engineered into the hinge region has been described, e.g., in U.S. Pat. No. 5,677,425 and Carter, et al.,Bio Technology, Vol 10, February. 1992, pages 163-167 the disclosures of which are hereby incorporated by reference. That thiol site can be used for conjugation to moieties such as polyethyleneglycol (PEG). Pegylation technology is known, for example, see Koumenis et al, Int. J. Pharm. (2000) 198(1): 83-95, incorporated herein by reference, which makes it possible to link two Fab′ molecules together using PEG coupling.

Technology for bacterially producing Fab′2 involves cloning the human IgG hinge region, and optionally part of the CH2, as part of the Fd which includes additional cysteines and is described, e.g., in Better, et al., PNAS USA (1993) 90(2): 457-61, incorporated herein by reference. The additional thiol groups on the Fd hinge can interact and cause two Fab′ molecules to dimerize, creating a Fab′2. Fab′2 can be purified directly from the bacterial cells. Additionally, undimerized Fab′ from the bacteria can be isolated and chemically converted to Fab′2.

As described earlier, the variable regions of the antibody can be cloned as a single chain wherein the variable light (VL) is connected to the variable heavy (VH) by a linker region. If that linker region is short (for example 5-7 amino acids), the folding of the scFv will favor association of two scFvs where the VL of one scFv paris with the VH of the second scFv. In this manner, two antigen binding sites are presented on the diabody.

Antibody constructs which contain two binding sites may be generated using any of these methods in order to test agonist activity and/or be used as the final therapeutic product.

Following the panning or sorting steps of Fab libraries, the library of panned molecules are restricted with Sac I and Spe I and cloned into pRL8. Subcloning to pRL8 vector individually or en masse following FACS sorting or panning allows expression of dimeric soluble binding Fabs for analysis in bioassays. In pRL8, the antibody fragments are transported to the periplasmic space and form dimers there. The advantage of this approach is that it permits panning of monomeric Fab fragments, favoring high affinity Fabs.

Another approach uses a secondary antibody. pRL4 has the hemagglutinin decapeptide tag recognized by the commercially available HA. 11 antibody (Babco, Berkeley, Calif.). Fabs identified in FACS sorting or panning to be tested in bioassay are preincubated with HA. 11 which will promote dimerization, prior to addition to bioassays.

Once binding scFv's or Fabs are identified by panning or another selection method, the individual clones, each expressing a unique dimerized antibody fragment on the phage surface are tested for proliferation, differentiation, activation or survival effects on target cells. In addition, soluble dimerized antibody are examined in bioassays.

Biological Assavs for Screening for TPO-like Activity

1. Colony formation assays—Megakaryocytic colonies from bone marrow (Megacult C Kit from Stem Cell Technologies Inc., Vancouver BC, Canada).

2. Proliferation assays—proliferation of Ba/F3 cells (Cwirla et al. 1997, Science, Vol. 276 pages 1696-1699). The Ba/F3-mpl cell line was established (F. de Sauvage et al., Nature, 369:533 (1994)) by introduction of the cDNA encoding the entire cMpl receptor into the IL-3 dependent murine lymphobiastoid cell line Ba/F3. Stimulation of proliferation of Ba/F3-mpl cells in response to various concentrations of antibodies or TPO was measured by the amount of incorporation of ³H-thymidine as previously described (F. de Sauvage et al., supra).

3. Phosphorylation assays—phosphorylation of JAK2 (Drachman et al., J. Biol. Chem., (1999), Vol. 274, pages 13480-13484).

4. Transcriptional based assays—Transiently co-transfect full length cMpl receptor with c-Fos promoter luciferase reporter construct. 24 hour post transfection starve the cells in 0.5% FCS for 24 hours. Stimulate the cells, harvest after 6 hours and take luciferase readings (see also Example 1, Biological Assays section).

Biological Assays for Screening for EPO-like Activity

1. Bone marrow erthroid colony formation in Methylcellulose (Wrighton et al., Science, 1996, Vol. 273 pages 458-463).

2. TF-1 cell (Human erythroleukemia cell line) proliferation. TF-1 cells express both full length and a truncated form of the Epo-R. (J.Cell Physiol., 1989, Vol 140, pages 323-334).

3. The EPO receptor couples directly to JAK2 kinase to induce tyrosine phosphorylation. Epo induces cFos in TF-1 cells. c-Fos transcriptional activation. (Witthuhn et al., Cell, (1993), Vol. 74, pages 227-236).

Biological Assays for Screening for hBNP-like Activity

Functional screening of isolated clones is conducted using a cell based assay system for the evaluation of human brain natriuretic peptide (hBNP) activity on natriuretic peptide receptor type A (NPRA)-bearing cells (neuroblastoma cell line, SK-N-SH).

Biological Assays for Screening for GLP-1-like or Exendin-like Activity

Functional screening of insulinotropic activity of isolated clones can be conducted by:

1. Rat pancreas perfusion experiments or stimulation of cyclic AMP production evaluated using cultured RINm5F insulinoma cells using the methods described in Watanabe, et al., J Endocrinol. Jan;140(1):45-52 (1994); Gallwitz ey al., J Mol Endocrinol. Jun;10(3):259-68 (1993); Richter, et al., J Endocrinol., Sep;126(3):445-50 (1990); Gallwitz et al., J Mol Endocrinol., Aug;5(1):33-9 (1990); Flatt et al., Diabetes Res., Feb;13(2):55-9 (1990); and/or Goke, J Endocrinol., Mar;116(3):357-62 (1988).

2. Evaluating binding affinity to GLP-1 receptor using techniques known to those skilled in the art.

Biological Assays for Screening for Adiponectin-like Activity 1. Oleate oxidation in isolated muscles and in mouse C2C12 skeletal muscle cells and Hepa-1-6 hepatocytes as described in Fruebis, et al., Proc. Natl. Acad. Sci. USA 98(4): 2005-10 (2001).

2. Cell proliferation assay using human aortic smooth muscle cells (HASMCs) (Clonetics) and human aortic endothelial cells (HAECs) (Clonetics) with or without PDGF-BB, HB-EGF, FGF, and EGF as described by Matsuda, et al., J. Biol. Chem. 277(40): 37487-91 (2002).

3. Inhibition of TNF-α-induced THP-1 adhesion and expression of VCAM-1, E-selectin, and ICAM-1 on HAECs as described by Ouchi, et al., Circulation 100 (25): 2473-6 (1999).

4. Binding to PDGF-BB and suppression of PDGF-BB-induced proliferation of HASMCs as disclosed by Arita, et al., Circulation 105 (24): 2893-8 (2002).

A number of bioassays can be used in high-throughput screening. Those of ordinary skill in the art are familiar with these and other suitable bioassays. Several non-radioactive assays have been developed in which either DNA synthesis or enzyme activity can be analyzed. For example, an MTT cell proliferation assay (Promega Corporation, Madison, Wis.) that is based on an assay described by Mosmann (Mossman, T. 1983. J. Immunol. Methods 65:55-57 incorporated herein by reference) can be used. This protocol is fast and easy. In the assay, MTT (3-[4,5-dimethylthiazol-2-yl]-2,5-diphenyl-tetrazolium bromide), a tetrazolium salt, is converted into a blue formazan product by mitochondrial dehydrogenase activity in living cells. The dehydrogenase content, and therefore the amount of colored product produced, is proportional to cell number. The colored product is detectable in an ELISA plate reader at 570nm. Assays are performed in triplicate, en masse in 96 well microtiter plates. Briefly, target cells are plated in 100 μl aliquots in culture medium in 96-well plates. Following addition of various concentrations of antibodies, cells are incubated for 48-72 hours at 37° C. and 5% CO₂ in a fully humidified atmosphere. MTT is added to each well, and proliferation monitored via ELISA plate reader.

For example, in proliferation assays using TF-1 cells, bacterial cells containing phagemids expressing antibodies are grown overnight at 37° C. in 96 well deep well plates in 1 ml of a media that is a mixture of mammalian cell media and bacterial media (in the case of TF-1 cells: RPMI 2.7/SB 0.3/Carb 100ug/ml). TF-1 cells are a human bone marrow erythroleukemia cell line that responds to multiple cytokines (Kitamura, T., Tange, T., Terasawa, T., Chiba, S., Kuwaki, T., Miyagawa, K., Piao, Y. F., Miyazono, K., Urabe, A., Takaku, F., Cell Physiol. 140:323-334,1989; Kitamura, T., Tojo, A., Kuwaki, T., Chiba, S., Miyazono, K., Urabe, A., Takaku, F., Blood 73:375-380,1989; Kitamura, T., Takaku, F., Miyajima, A., Int. Immunol. 3:571-577, 1991) On the following day, the overnight cultures are subcultured 1/10 to fresh trays, and placed at 37° C. for 2 hours. Following induction with IPTG at 37° C. for 4 hours, the plates are centrifuged at 2000 rpm/15′ at room temperature. 50 ul each culture supernate are filtered in 96 well filter trays (Millipore) to sterile 96 well assay plates. Mammalian cells are prewashed to remove growth factor and resuspended at a concentration of 1×10⁵ cells/ml. 50 ul cells are added to each well. Assay plates are incubated in 37° C./5% CO₂ incubator for 72 hours. At 72 hours, the trays are developed by adding 40 ul media/MTS/PMS per well. MTS is an improved more soluble version of MTT. Both assays are based on the cellular conversion of tetrazolium salt. A MTS proliferation assay kit (catalogue number G5421) can be purchased from Promega, Inc. (Madison, Wis.). Plates are kept at 37°/CO₂ incubator and read at OD₄₉₀ at 1 hour, 4 hours, 8 hours with microplate reader.

The activities of cytokines are often synergistic. Synergy could be manifested through the binding of ligands to two different receptors which then sends the correct signal, or via a priming effect whereby interaction of ligand/receptor primes the cell to respond to a second cytokine. Furthermore, cytokines that act early in lineage development are more often synergistic than cytokines that act at later stages in a developmental pathway. Therefore, suboptimal concentrations of growth factors can be used in these bioassays to examine synergism. Conditions for suboptimal concentrations are determined for each assay. This is done by adding serial dilutions of growth factors, individually and as a mixture, to the assays and determining the levels below which a single factor does not promote a response compared to the mixture, and the level below which the mixture does not promote a response in the bioassay. Bone marrow stromal cells can also be added in bioassays to provide other necessary factors that may play a role in a synergistic response.

In addition, cell proliferation can be examined by monitoring DNA synthesis. A non-radioactive, colorimetric assay that examines 5-bromo-2′-deoxy-uridine (BrdU) incorporation (Roche Molecular Biochemicals, Indianapolis, Ind.) can be performed in microtiter plate format. Here, cells are cultured in 96-well plates and incubated with BrdU and sub-optimal concentrations of cytokines. The amount of BrdU is determined after labeling with a peroxidase labeled anti-BrdU antibody. Final results are analyzed by ELISA plate reader at 405 nm.

A radioactive mitogenesis assay that measures the rate of DNA synthesis as an indication of proliferation (Raines and Ross, Methods of Enzymol. 109: 749-773, 1985) can also be used. In these assays, changes in rate of incorporation of [³H]thymidine in target cells is examined. Again, these assays permit concurrent and rapid screening of many antibody fragments. They have been widely used as a convenient method of assessing the stimulatory and inhibitory effects on the growth of many different cells. Cells are cultured in suspension until they reach exponential growth rate. Cells are then washed free of the medium in which they were cultured, and replated in fresh medium. Cells are aliquoted into 96 well plates in a total volume of 100 ul at a concentration of about 1-2×10⁵ cells/ml. Dilutions of phage supernatant, soluble dimerized Fab or ScFv antibodies are added and cells are incubated for 18-48 hours in a gassed CO₂ incubator at a temp of 37° C. Following incubation, [³H]thymidine (937 kBq) is added to each well and incubated for a further 4 hours. The cells are then removed from the incubator and counted directly in a bench top microplate scintillation counter such as Packard Top Count NXT Instrument (Packard, Meriden, Conn.). Alternatively cells can be serially transferred to GF/C filters on a Millipore cell harvester (Millipore, Bedford, Mass.) or similar apparatus. Radioactivity associated with acid-insoluble material retained on the filter is then determined. Dilutions of commercially available growth factors are applied to positive control wells. Negative controls would include supernatants from cells carrying non-insert containing plasmids or irrelevant antibodies treated similarly. The relative growth promoting activities of the standard and the diluents of the phage supernatants under test are compared to quantify the growth promoting activity in the sample.

Activation can be tested for by assaying second messengers or by transcriptional readout assays.

Survival can be assayed, for example, by monitoring apoptosis using assays such as tunnel assays or by other methods known to those who practice the art.

Other useful assays to analyze cellular signal transduction, the activity of kinases and phosphatases and ultimately cellular activities as a result of agonist activity include measurement of the generation of second messengers, e.g. cAMP, Ca++, diacylglycerol (DAG), and inositol 1,4,5-triphosphate (IP3). Measurement of spikes in intracellular calcium concentration, intracellular pH and membrane potential in high throughput screening assays can be performed using instruments such as the FLIPR Fluormetric Imaging Plate Reader System (Molecular Devices, Sunnyvale, Calif.). A number of fluorescent probes are available for examination of second messenger concentrations (Molecular Probes, Eugene Oreg.). Measurement of concentrations of second messengers can also be done on the single cell level (DeBernardi, M. A. and Brooker, G. Proc. Natl. Acad. Sci USA 93:45774582, 1996). In addition, assays that examine other signaling events such as phosphorylation, apoptosis or levels of RNA or protein of specific genes would be useful. For example. most cytokines have been shown to activate the enzyme PI13-K (reviewed in Silvennoinen, O., Ihle, J. N. Signaling by the Hematopoietic Cytokine Receptors, R. G. Landes company, Austin, Tex. 1996). Furthermore, the Jak family of tyrosine kinases have been shown to be central mediators for cytokine receptor signaling (Ihle, J. N., Witthuhn, B. A., Quelle, F. W. Annu. Rev. Immunol. 13:369-398, 1995). In addition, several other tyrosine kinases, e.g., members of the Src family, are activated in response to certain cytokine stimulations. In the case of RNA or proteins, e.g., c-Jun and c-Fos are rapidly and transiently upregulated upon cytokine stimulation, while c-Myc induction is slower. These proteins are required for G1 transition and proliferation (reviewed in Silvennoinen, O., Ihle, J. N. Signaling by Hematopoietic Cytokine Receptors, R. G. Landes Company, Austin, Tex. 1996). High throughput screens that detect increases in these transcripts could be utilized.

In transcriptional read out assays, changes in the transcription of specific genes are observed following exposure of cells to a growth factor′ or growth factor mimetic (agonist or inhibitory antibody). For example, in a myc read-out assay, cells such as IL-3 dependent FDCP-mix cell line is starved of IL-3 growth factor for 8 hours, followed by exposure to growth factor mimetics, or native growth factors for 2 hours at 37° C. At this time, the cells are harvested, RNA is isolated, and reverse transcriptase-polymerase chain reactions (RT-PCR) are performed with primers specific for the myc gene. The RT-PCR reactions are electrophoresed in horizontal agarose gels for quantitation of PCR product. In this case expression of a single gene is being monitored.

Alternatively assay for changes in expression of genes can be monitored using CHIP technology, agonist antibodies could be identified under conditions of high probe sensitivity and a dynamic range. In this way, up to 10,000 or more could be analyzed for changes in expression. Desired genes that could be monitored could include c-myc, c-jun, NF-κB, among others. These genes are downstream of various signal transduction pathways and their expression should change upon a mitogenic response. In one type of commercially available CHIP (Affymetrix, Santa Clara, Calif.), oligonucleotides from desired test genes can be printed out onto glass surface. Target cells are exposed to test agonist antibodies. RNA is isolated from the cells exposed to test agonist antibodies, copied to cDNA, and in vitro transcribed in the presence of biotin. Hybridization of in vitro transcribed, biotinylated mRNA is used as probe in the arrays. Chips are then scanned to determine genes that show increases in transcription upon exposure to test agonist antibodies. In another version of CHIP technology (Incyte, Palo Alto, Calif.), the amount of DNA is not normalized on the glass, therefore, one would set up a competitive hybridization. RNA is isolated from the cells before and after exposure to agonist. cDNA is made from each sample whereby one cDNA reaction has one label incorporated, for example, Cy-3, and the other cDNA population has a different label incorporated, for example Cy-5. Signals are detected and compared on a dual laser scan to collect images.

Visual assays can also be used such as traditional methylcellulose colony forming assays (Stem Cell Technologies, Vancouver BC, Canada). In these assays, colony growth, and morphological changes are scored via light microscope. Visual examination for proliferation or differentiation effects in semi-solid agar cultures or methylcellulose can be performed using the appropriate cell line. Williams Hematology 5 (eds. E. Beutler, M. A. Lichtman, B. S. Coller L T. J. Kipps), McGraw-Hill, Inc., pp L22-L26, 1995). Addition of methylcellulose allows clonal progeny of a single progenitor cell to stay together and facilitates the recognition and enumeration of distinct colonies. All necessary components are added to a basic methylcellulose medium (such as Iscove's MDM, BSA, (-mercaptoethanol, L-glutamine) except colony-stimulating factor supplements and test antibodies (phage supernatants, soluble antibodies) are added to see if they can substitute for growth factors. Cells in methylcellulose culture are incubated for 10-12 days following the addition of antibodies in a 37° C. humidified atmosphere of 5% CO₂ in air. After 10-12 days of incubation, colonies are counted using an inverted microscope. After another 8-10 days, colonies are counted again. Comparisons are made between media containing antibodies and controls with and without growth factors. In addition, colonies can be picked from methylcellulose and individual cells examined cytologically by staining with Wright's stain (see Atlas of Hematological Cytology, F. G. J. Hayhoe and R. J. Flemans, Wiley-InterScience 1970).

The receptor-binding affinities of antibody fragments can be calculated (Lfas & Johnson, 1990) from association and dissociation rate constants measured using a BIACORE surface plasmon resonance system (Pharmacia Biosensor). A biosensor chip is activated for covalent coupling of gD-mpl receptor using N-ethyl-N′″-(3-dimethylaminopropyl)-carbodiimide hydrochloride (EDC) and N-hydroxysuccinimide (NHS) according to the supplier's (Pharmacia Biosensor) instructions. gD-mpl is buffer-exchanged into 10 mM sodium acetate buffer (pH 4.5) and diluted to approximately 30 μg/mL. An aliquot (5 μl) is injected at a flow rate of 1 μL/min to achieve approximately 400 response units (RU) of coupled protein. Finally, 1 M ethanolamine is injected as a blocking agent. For kinetics measurements, 1.5 serial dilutions of antibody are injected in PBS/Tween buffer (0.05% Tween-20 in phosphate buffered saline) at 25° C. using a flow rate of 20 μL/min. Equilibrium dissociation constants, K_{d} s, from SPR measurements are calculated as k_{off}/Kₒₙ. Standard deviations, sₒₙ for kₒₙ and s_{off} for k_{off}, are typically obtained from measurements with >4 protein concentrations (kₒₙ) or with >7 protein concentrations (k_{off}). Dissociation data are fit to a simple AB→A+B model to obtain koff +/−s.d. (standard deviation of measurements). Pseudo-first order rate constant (ks) are calculated for each association curve, and plotted as a function of protein concentration to obtain kon +/−s.e. (standard error of fit).

For conversion of antibody clones into full IgGs, the coding regions for both the light and heavy chains, or fragments thereof, can be separately cloned out of a bacterial vector and into mammalian vector(s). A single vector system, such as pDR1 or its derivatives, can be used to clone both light and heavy chain cassettes into the same plasmid. Alternatively, dual expression vectors where heavy and light chains are produced by separate plasmids can be used. Mammalian signal sequences need to be either already present in the final vector(s) or appended to the 5′ end of the light and heavy chain DNA inserts. This can be accomplished by initial transfer of the chains into a shuttle vector(s) containing the proper mammalian leader sequences. Following restriction enzyme digestion, the light chain and heavy chain regions, or fragments thereof, are introduced into final vector(s) where the remaining constant regions for IgG1 are provided either with or without introns. In some cases where introns are used, primer design for PCR amplifying the light and heavy chain variable regions out of pRL4 may need to include exon splice donor sites in order to get proper splicing and production of the antibodies in mammalian cells.

With either vector expression system (single or dual plasmid), the production of antibody heavy and light chains can be driven by promoters that work in mammalian cells such as, but not limited to, CMV, SV40, or IgG promoters. Additionally, the vector(s) will contain a selectable marker for growth in bacteria (such as, but not limited to, ampicillin, chloramphenicol, kanamycin, or zeocin resistance). Selectable markers for mammalian cells (such as, but not limited to, DHFR, GS, gpt, Neomyocin, or hygromyocin resistance) may also be present in the IgG vector(s), or could be provided on a separate plasmid by co-transfection.

Those of ordinary skill in the art using known techniques would be able to synthesize antibodies in other organisms such as yeast, mammalian, insect, and plants (Carlson, J. R. and Weissman, I. L., Mol. Cell. Biol., 8:2647-2650,1988; Trill, J. J., Shatzman, A. R., Ganguly, S. Curr. Opin. Biotechnol. 6:553-560,1995; Hiatt, A., Cafferkey, R. Bowdish, K. Nature 342: 76-78,1989).

As stated previously, antibodies made in accordance with the disclosure herein provide increased half-life (duration of action) to the activity of small peptides or peptide mimetics such as the TPO mimetic described herein. In another aspect, the serum half-life of an antibody can itself be prolonged by making derivatives that are pegylated. See, e.g., Lee, et al., Bioconjug. Chem (1999) 10(6): 973-81, incorporated herein by reference. Another advantage, e.g., of the TPO mimetic antibody described herein is that normal TPO treatment may result in generation of TPO neutralizing antibodies in patients which interfere with the activity of a patient's naturally occurring TPO. The present TPO mimetic antibody substantially reduces the likelihood that a detrimental immune response will be produced toward native TPO because it has a different amino acid sequence.

The molecules encompassed by the claims can be used in diagnostics where the antibodies or fragments thereof are conjugated to detectable markers or used as primary antibodies with secondary antibodies that are conjugated to detectable markers. Detectable markers, include radioactive and non-radioactive labels and are well-known to those with skill in the art. Common non-radioactive labels include detectable enzymes such as horseradish peroxidase, alkaline phosphatase and fluorescent molecules. Fluorescent molecules absorb light at one wavelength and emit it at another, thus allowing visualization with, e.g., a fluorescent microscope. Spectrophotometers, fluorescence microscopes, fluorescent plate readers and flow sorters are well-known and are often used to detect specific molecules which have been made fluorescent by coupling them covalently to a fluorescent dye. Fluorochromes such as green flurorescent protein, red shifted mutants of green fluorescent protein, amino coumarin acetic acid (AMCA), fluorescein isothiocyanate (FITC), tetramethylchodamine isothiocyanate (TRITC), Texas Red, Cy3.0 and Cy5.0 are examples of useful labels.

The molecules can be used in cell isolation strategies such as fluorescence-activated cell sorting (FACS) if fluorescent markers are used, In fluorescence-activated cell sorting, cells tagged with fluorescent molecules are sorted electronically on a flow cytometer such as a Becton-Dickinson (San Jose, Calif.) FACS IV cytometer or equivalent instrument. The fluorescent molecules are antibodies that recognize specific cell surface antigens. The antibodies are conjugated to fluorescent markers such as fluorescein isothiocyanate (FITC) or Phycoerythrin (PE).

Magnetic sorting is also possible. In magnetic sorting procedures, the antibody is linked directly or indirectly to magnetic microbeads. Cells are precoated with antibodies that recognize cell surface molecules, e.g., receptors involved in proliferation, differentiation, activation or survival. The antibodies are attached to magnetic beads conjugated with a secondary immunoglobulin that binds to the primary antibody displaying the peptide, such as to the HA molecular tag engineered into each antibody. The cells are then removed with a magnet. Magnetic sorting can be positive selection where cells of interest are bound by the antibody and hence the magnet, or negative selection where undesired cells are isolated onto the magnet.

Alternatively, radiolabeled antibodies can be used for diagnostic purposes.

Antibodies and fragments thereof disclosed herein are useful for the amplification of a variety of clinically relevant cell types. Treatment can be in vivo or ex vivo. For example, agonist antibodies are useful to treat patients suffering from a deficiency in a cell population caused by disease, disorder or treatment related to for example suppression of hematopoiesis where less than the normal number of cells of a given lineage or lineages are present in a patient. The following represent only some examples of the conditions that can be treated with the antibodies containing biologically active peptides disclosed herein, those who practice the art would be able to identify other diseases and conditions that would benefit from such treatment. For example, HIV-infected patients, patients undergoing chemotherapy, bone marrow transplant patients, stem cell transplant patients, and patients suffering from myeloproliferative disorders show subnormal levels of specific hematopoietic lineages.

Thrombocytopenia can be a result of chemotherapy, bone marrow transplantation or chronic disease such as idiopathic thrombocytopenia (ITP) which all result in low platelet levels. The present TPO mimetic antibodies can be used to treat such patients.

Patients undergoing renal dialysis often suffer from treatment related anemia with subnormal levels of red blood cells. In aplastic anemia, bone marrow suppression can cause pancytopenia or may affect only the red blood cells, the white cells, or the platelets. The disclosed antibodies will augment the armamentarium of therapeutic agents for these and other diseases and disorders characterized by deficiencies in specific cell populations, such as hematopoietic cells.

The molecules encompassed by the present disclosure can also be used for ex vivo proliferation and differentiation of cells. This is useful for gene therapy purposes, for example for traditional viral vector approaches, and for autologous bone marrow transplants.

In addition, certain antibodies in accordance with the present disclosure can be radiolabeled for radioimmunotherapy or conjugated to toxins to deliver such toxins to specific cell types and result in the killing of those cells.

A biologically active c-mpl agonist antibody capable of stimulating proliferation, differentiation and maturation of hematopoietic cells may be used in a sterile pharmaceutical preparation or formulation to stimulate megakaryocytopoietic or thrombopoietic activity in patients suffering from thrombocytopenia due to impaired production, sequestration, or increased destruction of platelets. Thrombocytopenia-associated bone marrow hypoplasia (e.g., aplastic anemia following chemotherapy or bone marrow transplant) may be effectively treated with the disclosed antibodies as well as disorders such as disseminated intravascular coagulation (DIC), immune thrombocytopenia (including HIV-induced ITP and non HIV-induced ITP), chronic idiopathic thrombocytopenia, congenital thrombocytopenia, myelodysplasia, and thrombotic thrombocytopenia.

The biologically active c-mpl agonist antibodies disclosed herein containing the TPO mimetic peptide may be used in the same way and for the same indications as thrombopoietin (TPO). Thrombopoietin (TPO) stimulates megakaryocytopoiesis and platelet production. These antibodies are expected to have a longer half-life than native or pegylated TPO and thus are used in indications where a longer half-life are indicated.

An example of an assay useful for determining activity of TPO mimetics is the rebound thrombocytosis assay which involves administering to mice a single injection of goat anti-mouse platelet serum to induce acute thrombocytopenia (day 0). On days 5 and 6 mice are injected with test samples. On day 8 platelet counts are determined (³⁵S incorporation into platelets).

EPO mimetic antibodies herein stimulate hematopoiesis in a manner similar to naturally occurring EPO. Such therapy is useful in treating conditions where red blood cell production is compromised such as in chronic renal failure. The biological activity of EPO mimetic antibodies may be determined using in vitro or in vivo assays.

One in vitro assay measures the effect of erythropoietin mimetic antibodies on erythropoiesis in intact mouse spleen cells according to the procedure of Krystal, G., Exp. Hematol. 11 :649-660 (1983). To screen various embodiments of the EPO mimetic antibodies for activity, for example, in vitro or in vivo, the EPO mimetic antibodies can be evaluated for the extent of erythropoiesis or receptor binding. Tests to determine biological activity are well-known to those of skill in the art. For example, the biological activity of erythropoietin can be measured as described in, e.g., U.S. Pat. No. 5,614,184 and U.S. Pat. No. 5,580,853 herein incorporated by reference.

In another aspect, this disclosure contemplates the treatment of congestive heart failure (CHF), either prophylactically or during CHF. According to the American Heart Association's 2001 Heart and Stroke Statistical Update, approximately 4.7 million Americans currently suffer from chronic congestive heart failure and 550,000 new cases of CHF will be diagnosed in the United States this year. In the early stages of CHF the body activates several hormonal pathways that help the heart compensate in the short-term, but have adverse long-term effects. These hormones, which include adrenalin, angiotensin II, aldosterone and endothelin, stimulate the heart to beat faster and stronger, thicken the wall of the heart and maintain blood pressure by constricting blood vessels and stimulating the kidney to retain sodium. If these pathways remain activated over a sustained period of time, the beneficial effects are lost and injurious effects develop, contributing to an eventual deterioration of heart function. Anitibodies in accordance with this disclosure can be used for treating CHF by regulating one or more of these hormonal pathways.

Antibodies having hBNP incorporated therein in accordance with this disclosure can be administered intravenously into acutely decompensated CHF patients, to exert diuresis, natriuresis, and vasodilatation in a dose dependent manner. The present hBNP-containing antibodies also regulate activity of the highly selective and specific natriuretic peptide receptor A (NPR-A) which has cytoplasmic guanylyl cyclase (GC) domains that are stimulated when the receptors bind a ligand as well as the more abundantly expressed receptor (NPR-C or C-type) which has a short cytoplasmic domain without GC activity.

For the treatment of acute decompensated CHF, the mimetic antibody can be administered in bolus injection as performed for BNP peptide. However, for the subsequent follow-up of the patient, continuous intravenous injection will generally not be necessary since the half-life of the antibody will be much longer compared with that of the peptide. Therefore, it may considerably shorten the hospitalization of the patients. For the treatment of chronic CHF patients, on the other hand, the use of BNP was not practical because of the short half-life of the peptide necessitating the continuous injection to be an effective treatment, therefore, leading to hospitalization. With the presently desctibed mimetic antibody, the interval between the injections can be prolonged. The patient can be treated in an outpatient facility and the number of episodes of decompensation and hospitalizations can be reduced.

In yet another aspect, the present disclosure contemplates methods of treating diabetes by administering antibodies having biologically active peptides incorporated therein. Thus, for example, in one embodiment, the antibody is engineered to contain the glucagon-like peptide (GLP)-1, a potent insulinotropic hormone. Administration of the GLP-1 containing antibody binds to the GLP-1 receptor thereby producing a glucose-dependent insulinotropic effect. Thus, in another aspect, the present disclosure contemplates methods to preserve or improve beta-cell function by administering an antibody containing GLP-1 to a patient susceptible to or afflicted with diabetes. In yet another aspect, the present disclosure contemplates methods to halt or delay the progressive deterioration of the diabetic state associated with type 2 diabetes by administering an antibody containing GLP-1 to a patient afflicted with type 2 diabetes.

The GLP-1 containing antibodies prepared in accordance with the present disclosure can also be administered to induce a dose-dependent and time-reversible endothelial-dependent relaxation of preconstricted pulmonary artery rings. The exendin antibodies described herein are agonists to the human islet GLP-1 receptor. The antibodies containing pituitary adenylate cyclase-activating polypeptide 38 (PACAP-38) also advantageously can be used in methods to treat diabetes since such an antibody potentiates and arouses beta-cell responses to glucose, thereby amplifying glucose-induced insulin secretion in islets.

The Adiponectin antibodies described herein can also be used as a diabetes treatment. Methods of increasing systemic insulin sensitivity by administering adiponectin antibodies are also contemplated. In another aspect, methods of increasing glucose uptake by muscle cells by administering antibodies having adiponectin incorporated therein are provided. In yet another aspect, administration of adiponectin antibodies prepared in accordance with the present disclosure decrease hepatic glucose output.

In addition, reduction of insulin resistance and hyperinsulinemia can be treated by administering the present adiponectin-containing antibodies, thereby reducing obesity and the development of diabetes. In particualrly useful embodiments Acrp30(C39S) or wild-type Acrp30 treated with dithiothreitol are engineered into antibodies in accordance with the present disclosure.

In another aspect, the present disclosure contemplates methods of improving insulin resistance, lowering blood sugar, and exerting antiatherogenic effects by administering antibodies engineered to contain thiazolidinedione derivatives such as synthetic PPAR (peroxisome proliferator-activated receptor)γ ligands to subjects afflicted with or susceptible to type 2 diabetes. Methods of regulating adiponectin expression and plasma concentrations in-mammals in vivo and in vitro using antibodies engineered to contain thiazolidinedione derivatives are also contemplated herein. In particularly useful embodiments, Rosiglitazone is the synthetic PPAR-γ agonist that is engineered into an antibody and used to increase plasma levels of adiponectin in subjects afflicted with type 2 diabetes.

The route of antibody administration is in accord with known methods, e.g., injection or infusion by intravenous, intraperitoneal, intracerebral, intramuscular, subcutaneous, intraocular, intraarterial, intrathecal, inhalation or intralesional routes, topical or by sustained release systems as noted below. The antibody is preferably administered continuously by infusion or by bolus injection. One may administer the antibodies in a local or systemic manner.

The antibodies in accordance with this disclosure may be prepared in a mixture with a pharmaceutically acceptable carrier. Techniques for formulation and administration of the compounds of the instant application may be found in “Remington's Pharmaceutical Sciences,” Mack Publishing Co., Easton, Pa., latest edition. This therapeutic composition can be administered intravenously or through the nose or lung, preferably as a liquid or powder aerosol (lyophilized). The composition may also be administered parenterally or subcutaneously as desired. When administered systematically, the therapeutic composition should be sterile, pyrogen-free and in a parenterally acceptable solution having due regard for pH, isotonicity, and stability. These conditions are known to those skilled in the art.

Briefly, dosage formulations of the compounds of the present disclosure are prepared for storage or administration by mixing the compound having the desired degree of purity with physiologically acceptable carriers, excipients, or stabilizers. Such materials are non-toxic to the recipients at the dosages and concentrations employed, and may include buffers such as TRIS HCI, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol.

When used for in vivo administration, the antibody formulation must be sterile and can be formulated according to conventional pharmaceutical practice. This is readily accomplished by filtration through sterile filtration membranes, prior to or following lyophilization and reconstitution. The antibody ordinarily will be stored in lyophilized form or in solution. Other vehicles such as naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

Pharmaceutical compositions suitable for use include compositions wherein one or more rationally designed antibodies are contained in an amount effective to achieve their intended purpose. More specifically, a therapeutically effective amount means an amount of antibody effective to prevent, alleviate or ameliorate symptoms of disease or prolong the survival of the subject being treated. Determination of a therapeutically effective amount is well within the capability of those skilled in the art, especially in light of the detailed disclosure provided herein. Therapeutically effective dosages may be determined by using in vitro and in vivo methods.

An effective amount of antibody to be employed therapeutically will depend, for example, upon the therapeutic objectives, the route of administration, and the condition of the patient. In addition, the attending physician takes into consideration various factors known to modify the action of drugs including severity and type of disease, body weight, sex, diet, time and route of administration, other medications and other relevant clinical factors. Accordingly, it will be necessary for the therapist to titer the dosage and modify the route of administration as required to obtain the optimal therapeutic effect. Typically, the clinician will administer antibody until a dosage is reached that achieves the desired effect. The progress of this therapy is easily monitored by conventional assays.

For any antibody containing a peptide, the therapeutically effective dose can be estimated initially from cell culture assays. For example, a dose can be formulated in animal models to achieve a circulating concentration range that includes the EC₅₀ as determined in cell culture (e.g., the concentration of the test molecule which promotes or inhibits cellular proliferation or differentiation). Such information can be used to more accurately determine useful doses in humans.

Toxicity and therapeutic efficacy of the antibody molecules described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Molecules which exhibit high therapeutic indices are preferred. The data obtained from these cell culture assays and animal studies can be used in formulating a range of dosage for use in human. The dosage of such molecules lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p.1).

Dosage amount and interval may be adjusted individually to provide plasma levels of the antibody which are sufficient to promote or inhibit cellular proliferation or differentiation or minimal effective concentration (MEC). The MEC will vary for each antibody, but can be estimated from in vitro data using described assays. Dosages necessary to achieve the MEC will depend on individual characteristics and route of administration. However, HPLC assays or bioassays can be used to determine plasma concentrations.

Dosage intervals can also be determined using MEC value. Antibody molecules should be administered using a regimen which maintains plasma levels above the MEC for 10-90% of the time, preferably between 30-90% and most preferably between 50-90%.

In cases of local administration or selective uptake, the effective local concentration of the antibody may not be related to plasma concentration.

A typical daily dosage might range from about 1 μ/kg to up to 1000 mg/kg or more, depending on the factors mentioned above. Typically, the clinician will administer the molecule until a dosage is reached that achieves the desired effect. The progress of this therapy is easily monitored by conventional assays.

Depending on the type and severity of the disease, from about 0.001 mg/kg to abut 1000 mg/kg, more preferably about 0.01 mg to 100 mg/kg, more preferably about 0.010 to 20 mg/kg of the agonist antibody might be an initial candidate dosage for administration to the patient, whether, for example, by one or more separate administrations, or by continuous infusion. For repeated administrations over several days or longer, depending on the condition, the treatment is repeated until a desired suppression of disease symptoms occurs or the desired improvement in the patient's condition is achieved. However, other dosage regimes may also be useful.

The present antibodies can also be used in diagnostic assays, e.g., for detecting expression of certain proteins in specific cells, tissues, or serum. Various diagnostic assay techniques known in the art may be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp.147-158). The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

The present antibodies also are useful for the affinity purification of proteins from recombinant cell culture or natural sources. In this process, the antibodies are immobilized on a suitable support, such a Sephadex resin or filter paper, using methods well known in the art. The immobilized antibody then is contacted with a sample containing the protein to be purified, and thereafter the support is washed with a suitable solvent that will remove substantially all the material in the sample except the protein, which is bound to the immobilized antibody. Finally, the support is washed with another suitable solvent that will release the protein from the antibody.

### EXAMPLES

The following examples are included for illustrative purposes only and are not intended to limit the scope of the invention.

### Example 1

### Library Construction Of TPO Mimetic Sequences Grafted Into A Human Antibody Framework

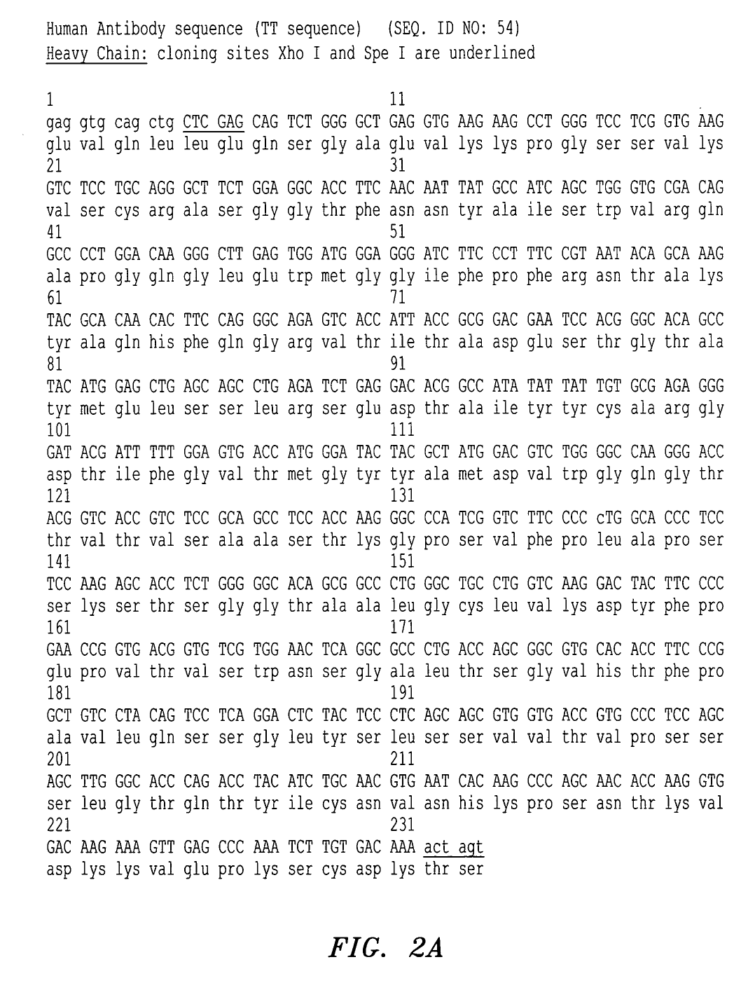

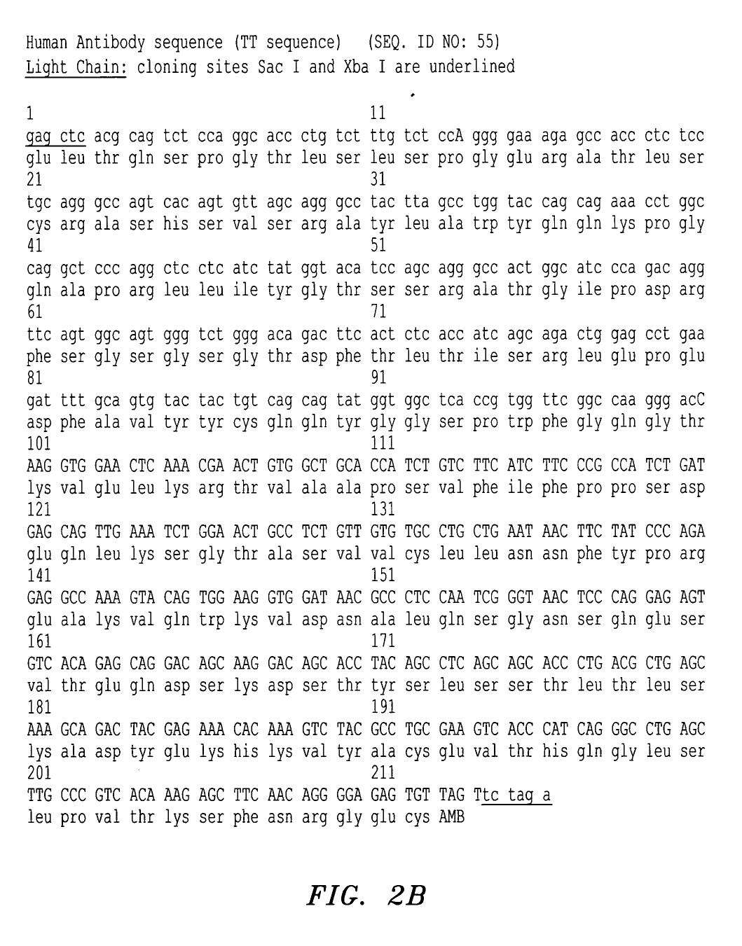

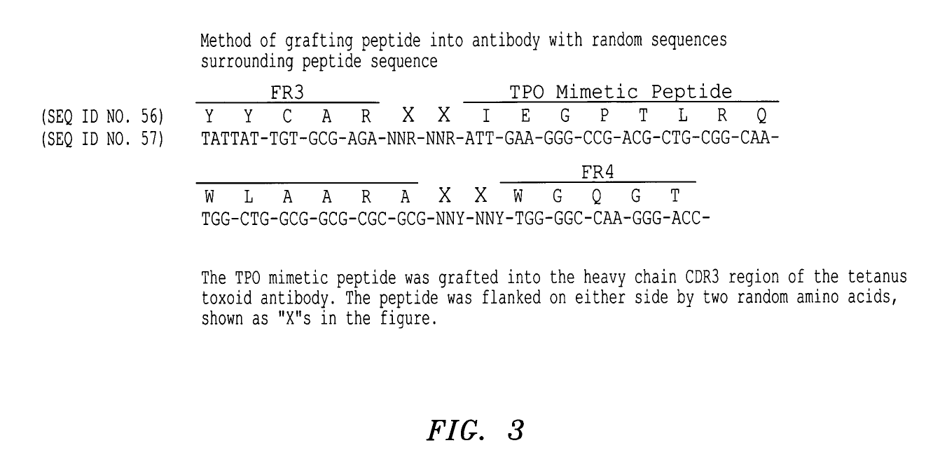

An agonist TPO mimetic-peptide IEGPTLRQWLAARA (SEQ. ID. NO: 1) was grafted into the anti-tetanus toxoid (TT) Fab heavy chain CDR3 (HCDR3), replacing the entire HCDR3 sequence GDTIFGVTMGYYAMDV (SEQ. ID. NO: 4). FIG. 2A shows the sequence for the human tetanus toxoid antibody employed. Two grafting approaches were taken. In the first approach the agonist peptide was inserted into the H-CDR3 region with two glycines flanking each side. This was to reduce structural constraints on the grafted peptide so that it could more easily adopt the needed conformation. In the second approach, two amino acid positions on each side of the peptide graft were randomized in order that the best presentation of the peptide could be achieved (FIG. 3). These two approaches were taken in order to determine whether the peptide alone was sufficient or if specific residues were required for proper presentation of the agonist peptide on the antibody scaffold, thereby conferring its activity to the antibody.

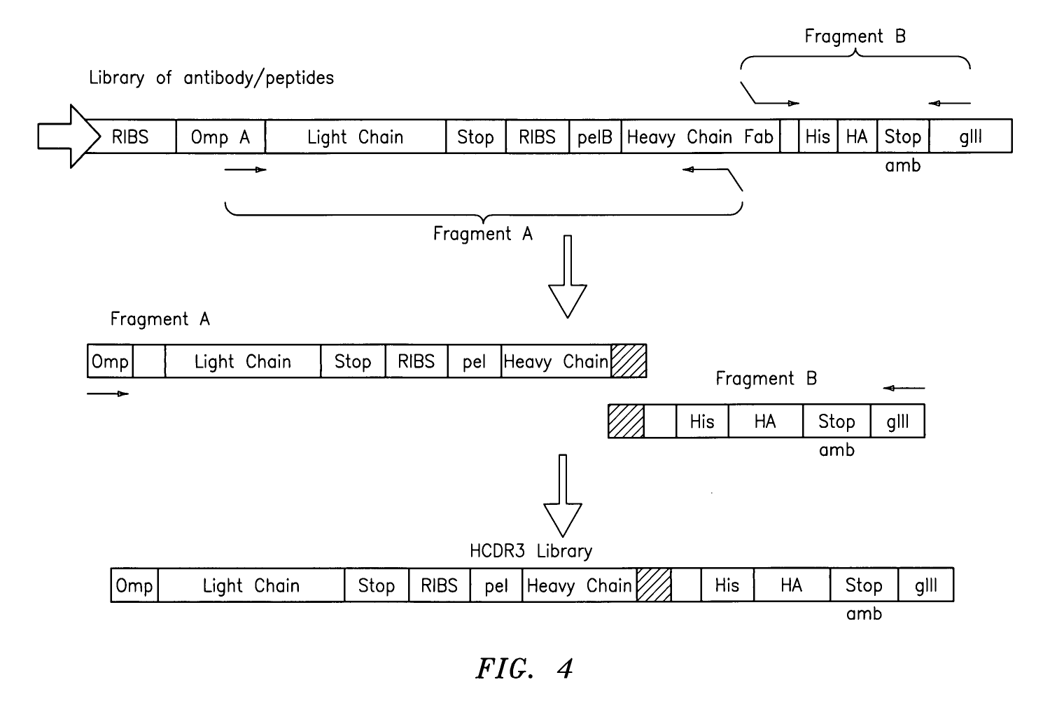

FIG. 4 outlines the library construction process. Briefly, the anti-tetanus toxoid Fab was amplified as two fragments. Fragment A was amplified using a forward primer (N-Omp: 5′ TAT CGC GAT TGC AGT GGC ACT GGC 3′)(SEQ. ID. NO: 5) that annealed to the Omp A leader for the light chain in combination with a backward primer (TPOCDR3-B: 5′ GC CAG CCA TTG CCG CAG CGT CGG CCC TTC AAT YNN YNN TCT CGC ACA ATA ATA TAT GGC 3′)(SEQ. ID. NO: 6) that annealed at the end of the heavy chain framework region (FR) 3. The reverse primer contained a tail encoding the new CDR3. Fragment B was generated using a forward primer (TPOCDR3-F: 5′ CCG ACG CTG CGG CM TGG CTG GCG GCG CGC GCG NNY NNY TGG GGC CAA GGG ACC ACC GT 3′)(SEQ. ID. NO:7) that annealed at the FR4 and the reverse primer Seq-G3Rev (5′ TCA AAA TCA CCG GM CCA GAG C 3′) (SEQ. ID. NO: 8) which annealed in the gene III region of the plasmid, downstream of the heavy chain stop signal. The TPOCDR3-F primer also had a tail of bases that encoded the new CDR3 region. TAQ DNA Polymerase (Perkin Elmer) was used in the following PCR program: 94° 30 seconds, then 30 cycles of 94° for 15 sec, 55° for 15 seconds, and 72° for 90 seconds, followed by an extension period at 72° for 10 minutes, and a hold at 4°. After the fragments were generated by PCR and gel purified, they were combined for an overlap extension PCR. The new CDR3 primer encoded regions were complementary and provided 23 bases of overlap. Primers N-Omp and SeqG3Rev were used in the overlap PCR protocol to generate the full Fab DNA product. Taq DNA Polymerase (Perkin Elmer) was used in the following PCR program: 94° 30″, then 20 cycles of 94° 30″, 56° 30″, and 72° 3′15″, then an extension period of 72° for 15′ followed by a 4° hold. After gel purification of the Fab product, an Sfi 1 digest was performed at 50° for 5 hours. Inserts were ligated into Sfi 1digested pRL4 vector overnight. Ligation products were ethanol precipitated, resuspended in H₂O, and then electroporated into competent ER2537 bacteria (suppressor strain, New England Biolabs). Following one hour of shaking in 5 mis SOC, an equal volume of SB was added. Carbenicillin was added to 20 ug/ml and the culture shaken for one hour at 37°, followed by one hour at 37° in 50ug/ml carbenicillin. The library culture was transferred into a flask containing 100 mis fresh SB, 50 ug/ml Carbenicillin, and 10¹² VCS M13 helper phage. After two hours at 37°, kanamycin was added to select for those bacteria that had been infected with helper phage. The following day, the overnight cultures were spun down and the phage in the supernate were precipitated on ice using 4% PEG/0.5 M NaCl. After spinning down the phage, the pellet was resuspended in 1 %BSA/PBS, filtered and dialyzed against PBS. Library phage were stored at 4°.

The construction of Fabs containing the non-randomly linked peptide was performed as described above by substituting primers TPOCDR3-B and TPOCDR3-F with alternate specific primers. For PP-(IEGPTLRQWLAARA)-GG (SEQ. ID. NO: 25) grafted antibody, primers used were TPOCDR3g-B (5′ GC CAG CCA TTG CCG CAG CGT CGG CCC TTC AAT NGG NGG TCT CGC ACA ATA ATA TAT GGC 3′)(SEQ. ID. NO: 9) and TPOCDR3g-F (5′ CCG ACG CTG CGG CM TGG CTG GCG GCG CGC GCG GGN GGN TGG GGC CAA GGG ACC ACC GT 3′)(SEQ. ID. NO: 10). For GG-(IEGPTLRQWLAARA)-GG (SEQ. ID. NO: 29) grafted antibody, primers used were TPO-CDR3-ggB (5′ GC CAG CCA TTG CCG CAG CGT CGG CCC TTC AAT NCC NCC TCT CGC ACA ATA ATA TAT GGC 3′)(SEQ. ID. NO: 11) and TPOCDR3g-F (5′ CCG ACG CTG CGG CAA TGG CTG GCG GCG CGC GCG GGN GGN TGG GGC CAA GGG ACC ACC GT 3′)(SEQ. ID. NO: 12).

Selection of the TPO Mimetic Peptide Heavy Chain CDR3 Library

In order to select for the optimal peptide display, panning was performed on human platelets. Because platelets express approximately 1800 TPO receptors per cell on their surface (cMpl receptors), they represented a good cell target. In addition, platelets are readily available from a local Blood Bank. To 1 ml of concentrated indated human platelets from the Blood Bank, 50 uls of freshly prepared Fab-phage were added in a 15 ml conical tube with 0.1% NaN₃. The tube was mixed at room temperature for 1-2 hours. Typically, 10 mls of 50% human serum (taken off the remaining platelets)+50%{IMDM/10% FBS/0.1% azide/2 mM EDTA} was added to the phage/cells. Platelets were pelleted at 5500×g for 5 minutes at room temperature. Supernatant was drained and the pellet was left resting under ˜500 uls of the wash for 20 minutes. The platelets were very gently resuspended and then 10 mis of 25% human serum (taken off the remaining platelets) +75%{IMDM/10% FBS/0.1% azide/2 mM EDTA} was added to the phage/cells. The centrifugation, pellet rest, and resuspension of the platelets was repeated. In panning rounds 3 and 4, a third wash was performed. The washed phage/cells were transferred to an eppindorf tube and spun at 5200×g. Phage were eluted from the platelets 10 minutes at room temperature using acid elution buffer (0.1M HCl, 1 mg/ml BSA, and glycine to pH 2.2). Platelets were pelleted at max speed and the eluted phage transferred to a 50 ml conical tube, neutralized with 2M Tris Base. Phage were then allowed to infect fresh ER2537 bacteria for 15 minutes at room temperature and amplified overnight as described above. Four rounds of platelet panning were performed.

After the fourth round of panning, pools of 3 Fab clones expressed as soluble proteins in nonsuppressor bacterial strain TOP10F′ (Invitrogen, Carlsbad, Calif.) were tested by Facs for binding to platelets by utilizing the Fabs′ HA epitope tag with rat high affinity anti-HA followed by anti-Rat-FITC (Sigma, St. Louis, Mo.). 25 uls indated concentrated human platelets (washed once with PBS/5 mM EDTA/2% FBS) were incubated with 100 uls bacterial supernate (60 uls bacterial supernate from three pooled Fab clones were pre-incubated with 40 uls 5% Milk/PSS at 4° for 15 minutes) at room temperature for 20-30 minutes. 1 ml of FACS buffer (PBS/2% FBS/5 mM EDTA) was added and cells spun down at 5200×g for 5 minutes. Pelleted cells were resuspended in 50 uls of 1:10 diluted (in PBS/1% BSA/0.1% NaN₃) 2° anti-HA antibody [Rat IgG anti-HA High Affinity clone 3F10 (Roche Molecular Biochemicals)] was added. After 30 minutes at room temperature, the cells were washed with 1 ml FACS buffer as above. Following centrifugation, cells were resuspended in 100 uls of 1:160 diluted (in PBS/1% BSA/0.1% NaN₃)3° anti-Rat IgG-FITC antibody (Sigma) and incubated 20 minutes at room temperature in the dark. Cells were washed with 1 ml FACS buffer then resuspended in 200 uls FACS buffer for analysis. As a positive control a commercially available sheep anti-IIb/IIIa Ab followed by anti-sheep FITC was used. Many pools of Fabs were clearly positive for binding to platelets by Facs. Follow up Facs analysis was then performed to identify individual clones that bound to the platelets.

Examination Of Individual Candidates Bv Binding Activity

Several Fabs, as bacterial supernatants, were tested for reactivity to the original antigen tetanus toxoid in order to determine if that binding specificity was retained. The antibody scaffold anti-TT Fab does bind to its antigen TT, but not to BSA. However, four TPO-mimetic peptide grafted Fab clones did not show significant binding to TT or BSA. As seen in previous experiments, the replacement of anti-TT Fab HCDR3 was sufficient to change the specificity of the antibody.

To further examine the binding capabilities of Fabs, Facs analysis was performed on CMK cells, a Megakaryocytic cell line (from German Collection of Microorganisms and Cell Cultures) which also expresses the cMpl receptor. Fab clones that bound CMK cells were then analyzed to verify that the platelet and CMK cell binding was occurring via the cMpl receptor. For that experiment, 293 EBNA cells were transfected with or without the cMpl-R, which had been cloned from Tf-1 cells by RT-PCR. 1×10⁶ transfected cells were incubated with bacterial supernate from each Fab clone (pre-blocked as described above) for 20-30 minutes at room temperature. Cells were spun down at 2000 rpm for 5 minutes. Pelleted cells were resuspended in 90 uls FACS buffer (PBS/2% FBS/1 mM EDTA) then 10 uls of 2° anti-HA antibody [Rat IgG anti-HA High Affinity clone 3F10 (Boehringer Mannheim Biochemicals) was added for a final 1:10 dilution. After 20 minutes at room temperature, the cells were washed with 1 ml FACS buffer. Following centrifugation, cells were resuspended in 100 uls of 1:50 diluted (in PBS/1% BSA/0.1% NaN₃) 3° anti-Rat IgG-PE antibody (Research Diagnostics Incorporated, RDI) and incubated 20 minutes at room temperature in the dark. Cells were washed with 1 ml FACS buffer then resuspended in 200 uls FACS buffer for analysis. Fabs selected during panning demonstrated strong binding to cells transfected with the cMpl-R but not to control vector transfected cells lacking the cMpl-R. This indicates that cell surface binding was occurring specifically through the cMpl receptor. Anti-TT Fab does not bind to control vector or cMpl-R transfected 293 cells. However, Fab clone X1c shows a shift from 3% binding of control (non-cMpl receptor) transfected cells to 95% binding of cells expressing the cMpl-R.

Examination Of Individual Candidates BY Sequence

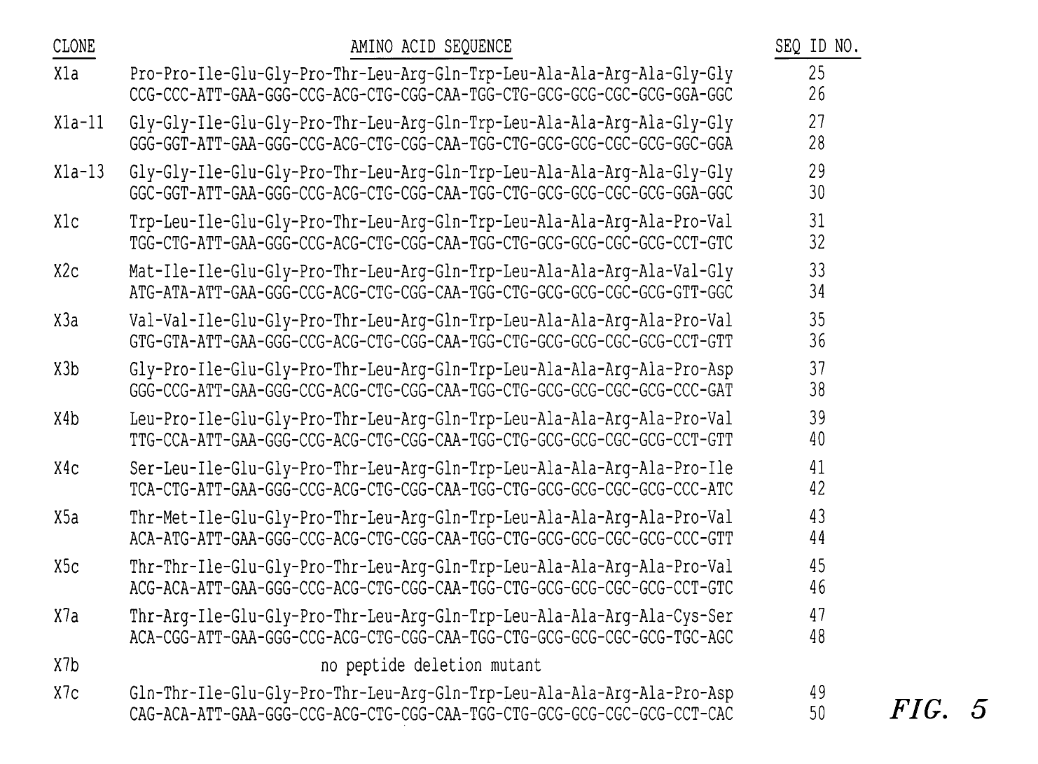

Sequence analysis of Fab clones which specifically bound to the cMpl receptor (see FIG. 5), revealed the selection of preferred amino acids at the downstream linkage site. The DNA sequence data was analyzed and the amino acid and DNA sequences are as follows:

!? Binding? SEQ.? ? ? !Clone? Properties? ID. NO? Sequence

| | | | |
|---|---|---|---|
| X1a | weak | 25 | Pro Pro (14 aa peptide) |
| | | | Gly Gly |
| X1a-11 | weak | 27 | Gly Gly (14 aa peptide) |
| | | | Gly Gly |
| X1a-13 | weak | 29 | Gly Gly (14 aa peptide) |
| | | | Gly Gly |
| X1c | strong | 31 | Trp Leu (14 aa peptide) |
| | | | Pro Val |
| X2c | weak | 33 | Met Ile (14 aa peptide*) |
| | | | Val Gly |
| X3a | strong | 35 | Val Val (14 aa peptide) |
| | | | Pro Val |
| X3b | strong | 37 | Gly Pro (14 aa peptide) |
| | | | Pro Asp |
| X4b | strong | 39 | Leu Pro (14 aa peptide) |
| | | | Pro Val |
| X4c | strong | 41 | Ser Leu (14 aa peptide) |
| | | | Pro Ile |
| X5a | strong | 43 | Thr Met (14 aa peptide) |
| | | | Pro Val |
| X5c | strong | 45 | Trp Leu (14 aa peptide) |
| | | | Pro-Val |
| X7a | weak | 47 | Thr Arg (14 aa peptide*) |
| | | | Cys Ser |
| X7b | weak | | deletion mutant this clone |
| | | | has lost the peptide |
| X7c | strong | 49 | Gln Thr (14 aa peptide) |
| | | | Pro Asp |

All clones which demonstrated strong binding, were found to contain a proline just downstream of the 14 amino acid TPO mimetic peptide. Selection by panning of a proline in the downstream linker position represents determination of a surprising amino acid choice which confers improved binding characteristics to the grafted TPO mimetic peptide. Weak binders did not contain this proline although they still contained the TPO mimetic peptide. It should be noted that clone X7a had a silent mutation in this peptide (GCG to GCA retaining the Ala at position 11 in the peptide) and that clone X2c had a mutation in this peptide switching a Thr for Ala at position 11 of the peptide.

Biological Assays

Clones were tested for agonist activity using a transcriptional based assay measuring luciferase activity driven by the c-Fos promoter. Dimerization of the cMpl receptor activates Jak which stimulates the MAP kinase pathway. Thus activation can be measured by assaying luciferase production and activity stimulated by MAP kinase via the cFos promoter. Since dimerization of the cMpl receptor is required for activation, either full IgG or dimerized Fab fragments capable of dimerizing the receptor, could be used to stimulate cMpl receptor activity. Fabs produced in bacteria were dimerized via the HA tag utilizing the 12CA5 anti-HA antibody. Increasing amounts of 12CA5 was added to the bacterial Fabs to dimerize the Fab clones in order that they might in turn dimerize and activate the cMpl receptor. For measurement of agonist activities Fab containing bacterial supernatants (2 mls) mixed with 12CA5 were applied to NIH3T3 cells which had been co-transfected with either a control vector or the cMpl receptor and the Fos promoter/luciferase reporter construct. Co-transfections of 3T3 cells were performed by plating NIH 3T3 cells at 3×10⁵ cells per 6 cm dish and then transfecting the following day. NIH 3T3 cells were transfected using the Effectine lipofection reagent (Qiagen), transfecting each plate with 0.1 ug pEGFP (a tracer to measure transfection efficiency), 0.2 ug of the Fos promoter/luciferase construct and 0.7 ug of either the empty control vector or the plasmid expressing the cMpl receptor. 3T3 cells were placed in 0.5% serum 24 hours post transfection and incubated for an additional 24 hours in this low serum media to reduce the background activation of the Fos promoter. Antibody supernatants were then applied to these cells for 6 hours. Cells were harvested and luciferase assays performed using 50 ug of cell lysate. No activation was stimulated by the antibodies in the absence of cMpl receptor expression. However, agonist activity was observed in cMpl receptor co-transfected 3T3 cells. This allowed us to demonstrate that the agonist activity observed was through the cMpl receptor and its interaction with the antibody. The data is as follows:

Bacterial Supernatants with 12CA5

| | Relative Fold Luciferase activity | |
|---|---|---|
| CELL Treatment | CMpl-R transfected | Control transfected |
| Untreated: | 1.0 | 1.0 |
| +10% FCS | 3.43 | 3.0 |
| +TPA | 2.3 | 2.42 |
| +TPO | 2.32 | 0.76 |
| +X4c Sup (alone) | 1.03 | 0.94 |
| +X4c + 60 ul α-HA | 1.97 | 0.84 |
| +X4c + 30 ul α-HA | 1.52 | 0.87 |
| +X4c + 10 ul α-HA | 1.22 | 0.86 |
| +X4c + 3 ul α-HA | 0.94 | 0.68 |
| +X4c + 1 ul α-HA | 0.91 | 1.05 |
| +X4c + 0.3 ul α-HA | 1.01 | 0.95 |

Activation of cMpl receptor can be tested in a similar manner using full IgGs (converted from Fab as described herein) produced by transient or stable transfection of mammalian cells rather than bacterially produced Fabs dimerized by anti-HA 12CA5. Experimentally transient transfection can be performed essentially as described here. For transfections 2×10⁶ cells (such as 293 EBNA) would be plated in 6 cm dishes for each test sample. The following day each plate would be transfected with 2.5 ug of total DNA (2 ug total of the light chain and heavy chain plasmid(s), 0.25 ug of pAdVAntage (Promega, Madison, Wis.), and 0.25 ug of pEGFP) using the Effectine reagent (Qiagen). The 293 cells would be placed in 0.5% serum 24 hours post transfection and incubated for an additional 24 hours in this low serum media to obtain full IgG. Residual growth factors are negligible in this media in stimulating receptors as seen in controls experiments. After 24 hours supernatants would be collected and spun for 5 minutes at 3000 rpm to remove any residual cells. For measurement of agonist activities of the full lgGs, 3mis of the conditioned 293 cell supernatants would be applied to NIH3T3 cells as described above.

### Example 2

### Additional Libraries Containing TPO Mimetic Sequences Grafted Into A Human Antibody Framework

Another approach to linking two agonistic peptides together in an antibody framework is to insert the agonist peptide in more than one position within a single Fab fragment. In order to do that, additional libraries containing TPO mimetic sequences grafted into a human antibody framework were constructed. Following selection of peptides properly presented in the context of CDR1, CDR2 or CDR3 of the light chain, or CDR2 of the heavy chain, the binding sequences were combined into a single Fab molecule, for example as listed in Table 1 below, and analyzed for enhanced activity.

TABLE 1

| Peptide 1 | Peptide 2 |
|---|---|
| H-CDR3 | H-CDR2 |
| H-CDR3 | L-CDR1 |
| H-CDR3 | L-CDR2 |
| H-CDR3 | L-CDR3 |
| H-CDR2 | L-CDR2 |
| H-CDR2 | L-CDR3 |
| L-CDR3 | L-CDR2 |

Four additional libraries have been constructed separately replacing the heavy chain CDR2 as well as the light chain CDR1, CDR2 and CDR3 with the TPO mimetic peptide flanked by 2 random amino acids using an NNK doping strategy. The generation of the libraries was similar to that described for the heavy chain CDR3 TPO peptide library, except that only the chain (heavy or light) being modified to form a library was amplified by PCR and used as the insert. PCR was performed using Expand High Fidelity PCR System (Roche) which contains a mixture of Taq and Pwo Polymerases. The first round of PCR was performed using the program: 94° 30″, then 30 cycles of 94°15 ″, 56° 30″, and 72° 2′, followed by elongation for 10′ at 72° and a 4° hold. Overlap PCR was performed for 10 cycles without primers using the program listed above to allow the full DNA template to be generated by the polymerases. Primers were then added to the PCR reaction tubes for 20 cycles of the same program for amplification.

For the HCDR2 library, the fragment A was created using the forward primer lead V_{H} (5′ GCT GCC CM CCA GCC ATG GCC 3′)(SEQ. ID. NO: 13), which annealed at the pel B leader signal located in front of the heavy chain, and the reverse primer HR2 CMPL ANTI (5′ AGC CAG CCA CTG GCG CAG GGT TGG GCC TTC GAT MNN MNN TCC CAT CCA CTC AAG CCC TTG 3′)(SEQ. ID. NO: 51) that annealed at the end of the heavy chain FR2. The reverse primer contained a tail encoding the new CDR2. Fragment B was created using forward primer HR2 cMpl CODE (5′ CCA ACC CTG CGC CAG TGG CTG GCT GCT CGC GCT NNK NNK AGA GTC ACC ATT ACC GCG GAC 3′)(SEQ. ID. NO: 14) which annealed at FR3 of the heavy chain and reverse primer N-dp (5′ AGC GTA GTC CGG AAC GTC GTA CGG 3′)(SEQ. ID. NO: 15) which annealed in the HA epitope tag region of the plasmid, downstream of the heavy chain constant region. The HR2 cMpl CODE primer also had a tail of bases that encoded the new CDR2 region. After the fragments were generated by PCR and gel purified, they were combined for an overlap extension PCR. The new CDR2 primer encoded regions were complementary and provided 24 bases of overlap. Primers leadVH and N-dp were used in the overlap PCR protocol to generate the full heavy chain DNA product. Following gel purification of the heavy chain product, a Xho I/Spe I digest was performed at 37° for 3 hours. Inserts were gel purified and then ligated into Xho I/Spe I digested pRL4 vector containing the anti-TT light chain. Ligation products were precipitated and electroporated into ER2537 bacteria as described above for the generation of the Fab-phage library.

The light chain CDR3 library was similarly made using primers for Fragment A of forward primer N-omp and reverse primer LR3 cMpl ANTI (5′ AGC CAG CCA CTG GCG CAG GGT TGG GCC TTC GAT MNN MNN ACA GTA GTA CAC TGC AAA ATC 3′)(SEQ. ID. NO: 16) and for Fragment B of forward primer LR3 cMpl CODE (5′ CCA ACC CTG CGC CAG TGG CTG GCT GCT CGC GCT NNK NNK TTC GGC CAA GGG ACC AAG GTG 3′)(SEQ. ID. NO: 17) and reverse primer leadB (5′ GGC CAT GGC TGG TTG GGC AGC 3′)(SEQ. ID. NO: 18). Primer leadB annealed to the pelB leader sequence located before the VH. The LR3 cMpl ANTI reverse and LR3 cMpl CODE forward primers annealed to the FR3 and FR4 of anti-TT light chain respectively. Both LR3 cMpl primers contain a tail of nucleotides encoding the new CDR3 peptide library, which provides the 24 basepair overlap region for the fusion PCR of Fragment A and Fragment B. Following purification of the light chain PCR products, a Sac I/Xba I digest was performed at 37° for 3 hours. The light chain fragments were then ligated into Sac I/Xba I digested pRL4 containing the anti-TT heavy chain overnight at room temperature. Ligation products were precipitated and electroporated into ER2537 bacteria as described above for the generation of the Fab-phage library.

The construction of the light chain CDR2 library was carried out as described above for the light chain CDR3 library with the exception that specific primers LR2 cMpl ANTI (5′ AGC CAG CCA CTG GCG CAG GGT TGG GCC TTC GAT MNN MNN ATA GAT GAG GAG.CCT GGG AGC 3′)(SEQ. ID. NO: 19) which annealed at the end of light chain FR2 and primer LR2 cMpl CODE (5′ CCA ACC CTG CGC CAG TGG CTG GCT GCT CGC GCT NNK NNK GGC ATC CCA GAC AGG TTC AGT 3′)(SEQ. ID. NO: 20) which annealed at the beginning of light chain FR3 were used in place of the LR3 cMpl primers.

The construction of the light chain CDR1 library was also carried out as previously described for the light chain CDR3 library with the exception that specific primers TPOLR1CODE

(5′ CCAACCCTGCGCCAGTGGCTGGCTGCTCGCGCTNNKNNKTGGTACCAGCAGA AACCTGGC 3′)(SEQ. ID. NO: 58) which annealed at the beginning of the light chain FR2 and the primer TPOLR1ANTI

(5′AGCCAGCCACTGGCGCAGGGTTGGGCCTTCGATMNNMNNGCAGGAGAGGGT GGCTCTTTC 3′)(SEQ. ID. NO: 59) which annealed to the end of the light chain FR1 were used in place of the LR3 cMpl primers.

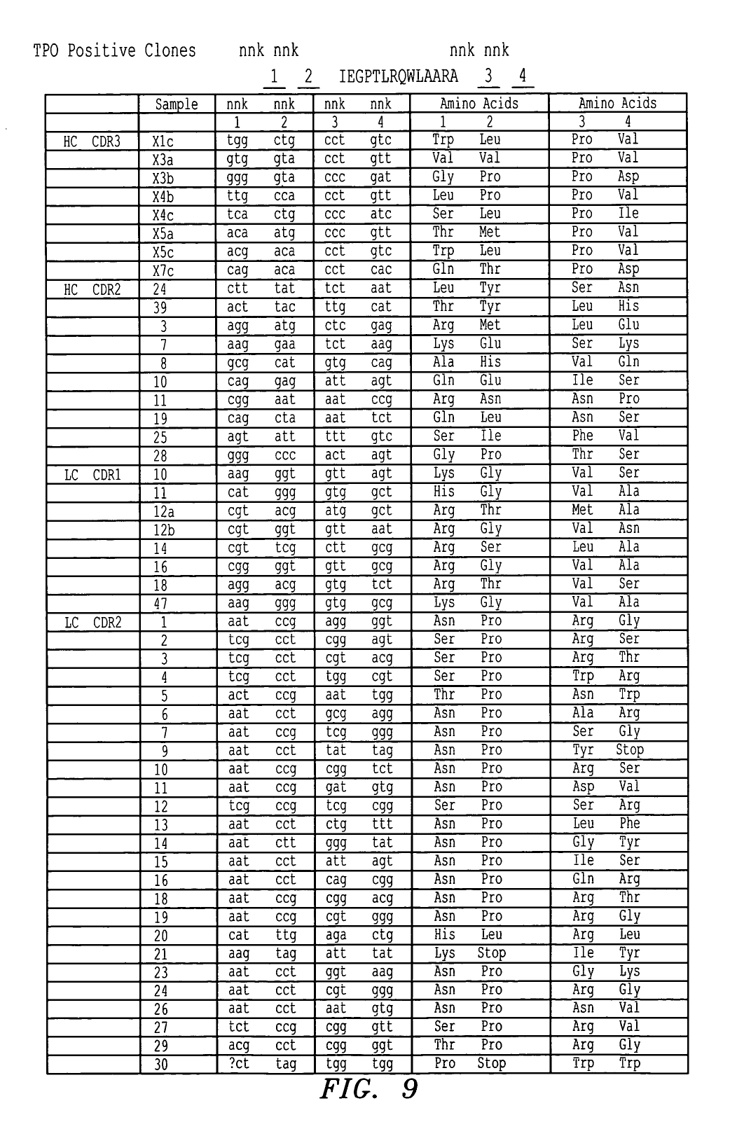

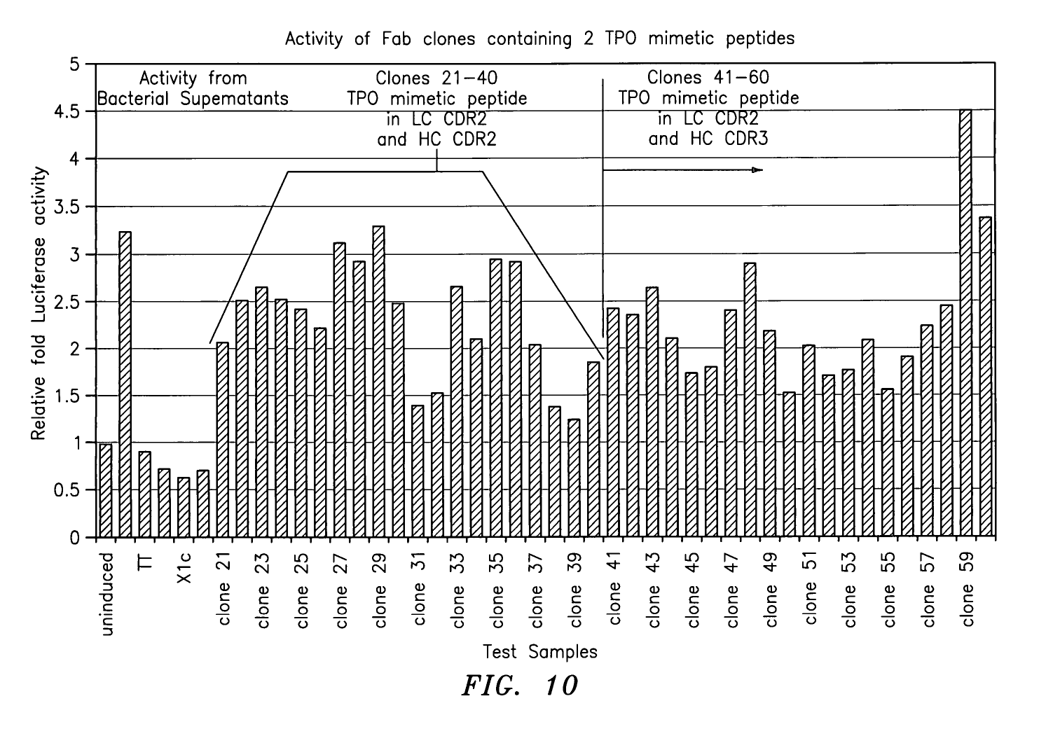

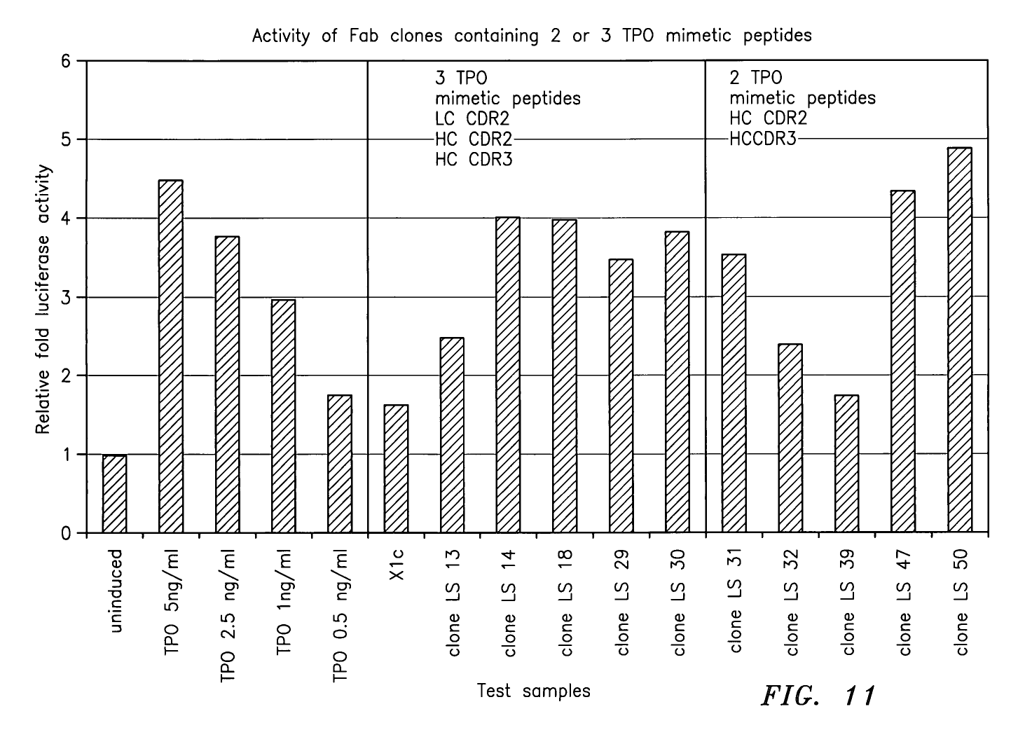

The three additional libraries, which separately replace the heavy chain CDR2 and the light chain CDR2 and CDR3 with the TPO mimetic peptide flanked by 2 random amino acids using the NNK doping strategy, were separately panned on platelets, as was previously described in Example I for the heavy chain CDR3 replacement library. Four rounds of panning were performed and clones were screened by FACS on platelets and cMpl receptor transfected 293 cells as previously described. Two positive clones were obtained from these screens. These clones had the TPO mimetic peptide in the heavy chain CDR2. Unlike with the heavy chain CDR3 clones neither of the heavy chain CDR2 clones had a proline in downstream position. Instead both were found to contain a tyrosine in the upstream position (See FIG. 9-clones HC-CDR2 No. 24 and No. 39).

The libraries, including LCDR1, were separately subjected to another panning experiment using cMpl receptor transfected 293 cells instead of platelets during the panning. The 293 cells were observed to reproducibly transfect at a high efficiency and express very high levels of the functional cMpl-receptor on their surface. Thus these cells represented a good cell target for use in panning. For these experiments different groups of plates of 293 cells were separately and sequentially transfected four days in a row. Each group of plates was then sequentially used for the four separate rounds of panning. Each round of harvesting of the cells and panning occurred two days after transfection. For harvesting, cells were removed from the plates using cell disassociation buffer, spun down at 1500 rpm for 5 minutes and re-suspended in IMDM supplemented with 10% FCS, 0.1% sodium azide and 5 mM EDTA at a concentration of 1×10⁶ cell per ml (3×10⁶ for LC-CDR1). In the round one pan, 3×10¹¹ phage from each library were separately applied to 2 ml of cells (6×10⁶ for LC-CDR1 and 2×10⁶ cells for all others) and rotated in a 15 ml conical tube for two hours at room temperature. Cells were washed twice using 10 mis of the IMDM/10% FCS/0.1% sodium azide/5 mM EDTA buffer. Phage were eluted in acid and amplified as previously described in Example 1. In round two 4×10⁶ cells (6×10⁶ for LC-CDR1) were used in 2 ml of buffer and 3×10¹¹ phage from the amplified round one eluted phage was combined with 3×10¹¹ phage from the un-panned library and added to the cells. Washing, elution and amplification proceeded similar to round one. In round three 4×10⁶ cells (6×10⁶ for LC-CDR1) were used in 2 ml of buffer and 3×10¹¹ phage from the amplified round two eluted phage were used. Cells were washed three times prior to elution. In round four, 4×10⁶ cells (6×10⁶ for LC-CDR1) were again used in 2 ml of buffer and 3×10¹¹ phage from the amplified round three eluted and amplified phage were used. Cells were again washed three times prior to elution. At least thirty individual clones were screened by FACS on cMpl receptor transfected 293 cells as previously described. 12 positive clones were obtained from the heavy chain CDR2 library and 25 positive clones were obtained from the light chain CDR2 library, and 14 positive clones were obtained from the light chain CDR1 library. Clones were further analyzed by DNA sequence. The selected flanking amino acid residues for the positive clones are depicted in the attached FIG. 9. It is of interest to note that the light chain CDR2 grafted Fabs have a strong selection for a proline (Pro) upstream of the TPO mimetic peptide.

### Example 3

Combinations of the TPO mimetic peptide grafted Fab clones from FIG. 9 have been generated. Thus a single antibody might contain multiple copies of the TPO mimetic peptide within a single light or heavy chain. Alternatively, both the light and heavy chains might contain peptide grafts giving multiple copies within a single Fab. Positive clones selected from the same CDR library were pooled. New libraries were constructed by combining the pool of one TPO mimetic peptide containing CDR with the pool of another. Combinations where one of the TPO mimetic peptides is in the light chain and the other is in the heavy chain are made using simple cloning techniques using the pooled plasmid DNAs, and the unique restriction sites flanking the heavy (Xho I-Spel) and light chains (Sac I-Xba I). For example, the plasmid DNA for the H-CDR3 peptide grafted heavy chains were combined and digested by Xho I and Spe I. The purified heavy chain inserts were ligated into the Xho I/Spe I digested plasmid containing the L-CDR2 grafts. The resulting library contained heavy chains with CDR3 peptide grafts and light chains with CDR2 peptide grafts. It should be understood that individual clones could also be combined rather that using pools of clones for the pairing of two peptide containing CDRs. For example, a single heavy chain clone with a CDR3 peptide graft was paired with several individual light chain CDR1 clones to create Fabs with multiple copies of TPO mimetic peptides.

Combinations where two TPO mimetic peptides were combined within a given heavy chain was performed using overlap PCR to generate the fragment for cloning. Two overlapping primers which bind between CDR2 and CDR3, and flanking primers, such as “N omp” and “lead B” primers from the light chain and “Lead VH” and “Ndp” primers for the heavy chain were used. For example, to combine H-CDR2 and H-CDR3 the first PCR was performed using lead VH (a primer that anneals in the vector at the heavy chain pelB leader signal) and a reverse primer annealing at FR3 using the H-CDR2 pooled plasmid DNA as the template. The sequence of that primer was 5′ CCA TGT AGG CTG TGC CCG TGG ATT 3′ (SEQ. ID. NO: 63). In a separate reaction, the pooled plasmids containing the H-CDR3 grafts underwent PCR with a forward primer annealing in FR3 (which is complementary to the above FR3- reverse primer) and N-dp (which anneals in the vector at an epitope tag sequence). The sequence of that primer was 5′ CCA CGG GCA CAG CCT ACA TGG AGC 3′ (SEQ. ID. NO: 64). The first PCR products were purified then combined in an overlap PCR reaction, where fusion of the two fragments occurred through the complementary FR2 sequences. The full heavy chain product was cloned by Xho I/Spe I into a plasmid containing the anti-tetanus toxoid light chain. In all, five classes of Fabs bearing multiple copies of TPO mimetic peptides were created as detailed in Table 2 below.

TABLE 2

| Peptide 1 | Peptide 2 | Peptide 3 |
|---|---|---|
| H-CDR3 | H-CDR2 | |
| H-CDR3 | L-CDR2 | |
| H-CDR2 | L-CDR2 | |
| H-CDR3 | H-CDR2 | L-CDR2 |
| H-CDR3 | L-CDR1 | |

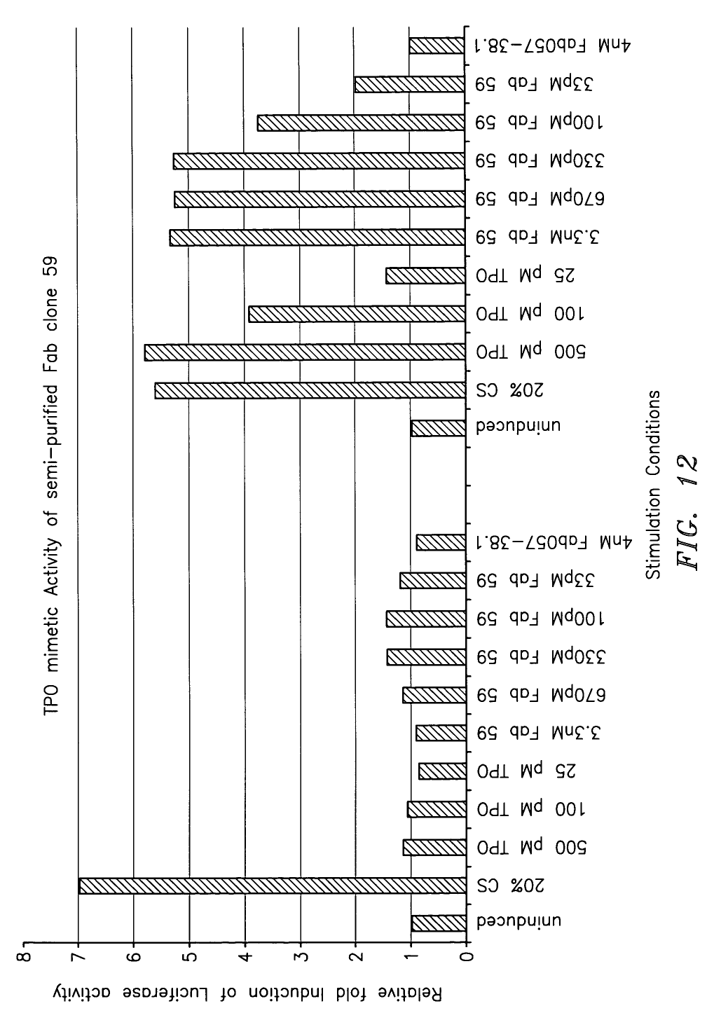

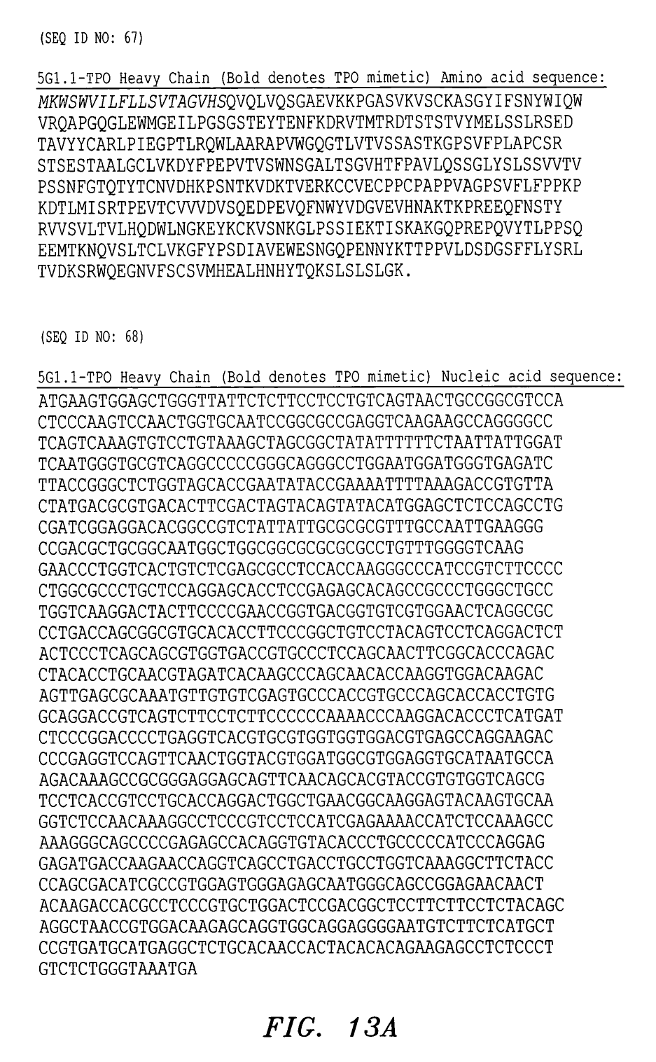

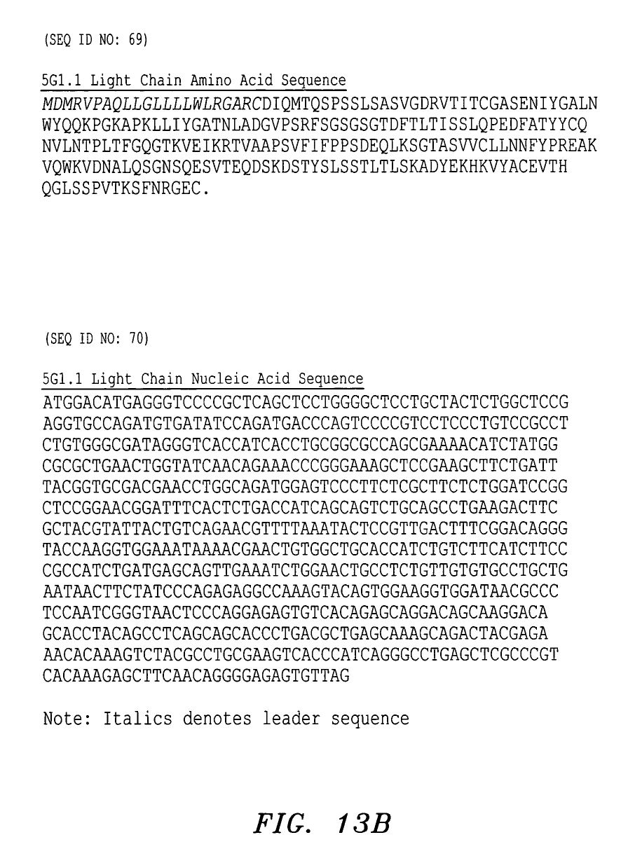

20 clones from each combination library and four individual clones of H-CDR3 plus L-CDR1 were tested for biological activity in a luciferase reporter assay as previously described. See FIGS. 10 and 11. In both experiments, the negative controls can include uninduced cells, cells treated with an irrelevant Fab (anti-tetanus toxoid), cells treated with a Fab clone that only weakly binds cMpl receptor, and X4b and/or X1c Fabs which do bind the cMpl receptor but only have a single binding domain and so can not activate the receptor. The positive control was the addition of TPO. All remaining samples were from the newly formed combination libraries. As can be observed, several clones have significant activity as Fabs. This indicates that incorporation of multiple TPO mimetic peptides into a single Fab molecule is able to bind two receptors and cause receptor activation.

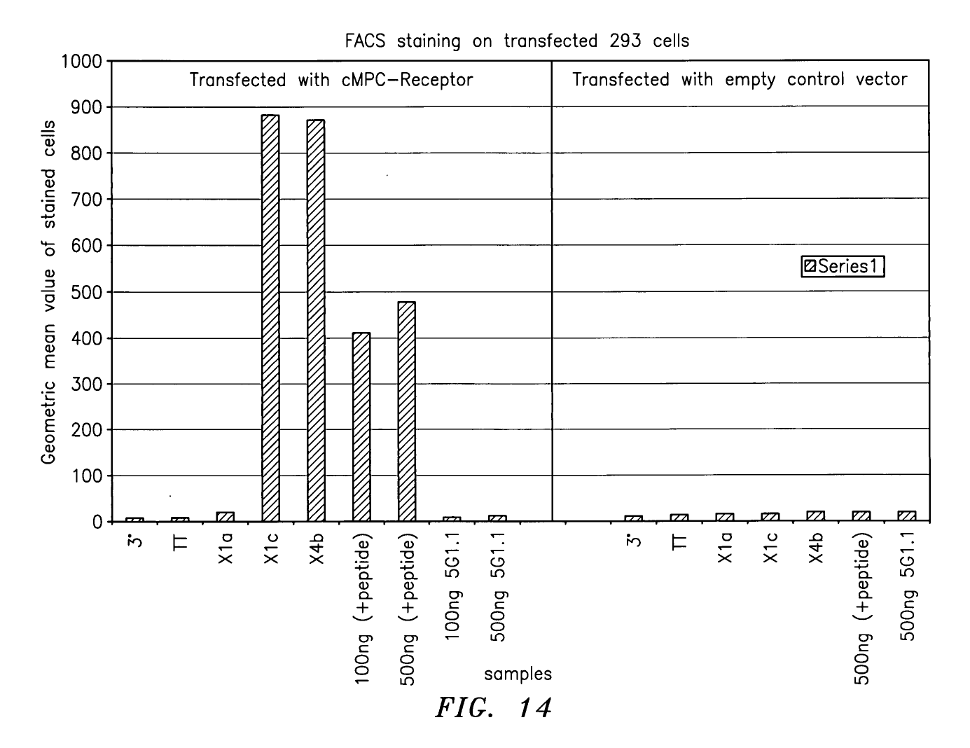

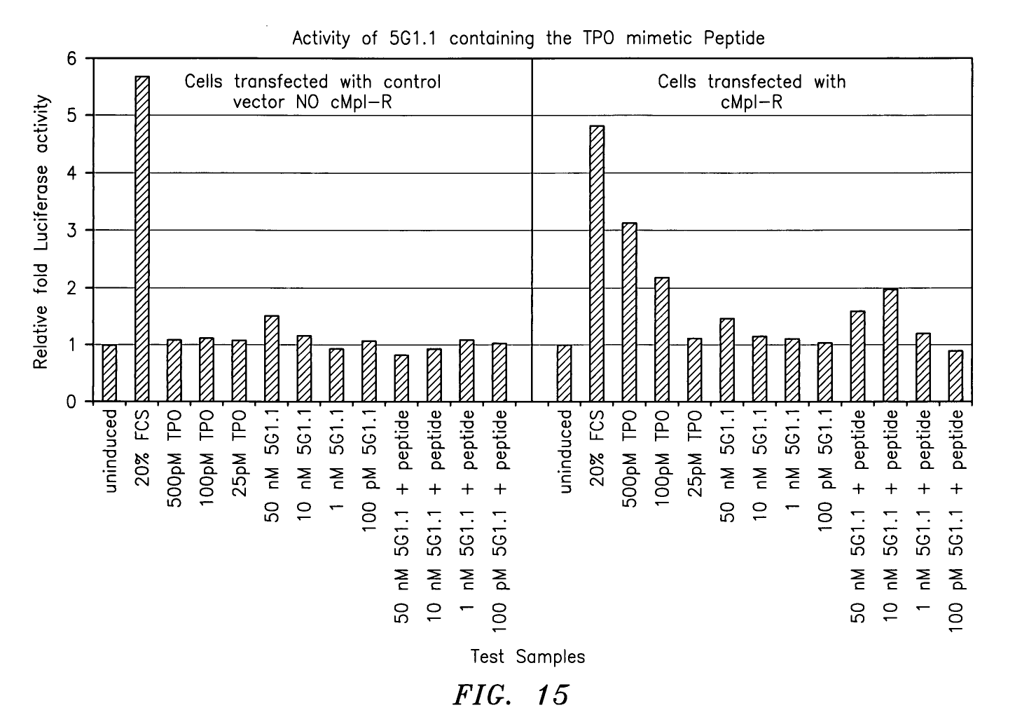

In fact, using Fab 59 the agonistic activity obtained can be as high as native TPO activity. Clone 59 containing two TPO mimetic peptides (HC CDR3 sample x4c and LC CDR2 sample 19 as identified in FIG. 9) and a His6 tag was partially purified from a bacterial periplasmic prep by FPLC using nickel column chromatography. The activity of this Fab was measured and found to be approximately equivalent to that of TPO (see FIG. 12), as estimated by cMpl-R specific induction of luciferase activity in direct comparison to known concentrations of TPO. A quantitative western blot was performed in order to determine the Fab 59 antibody concentrations.

These Fabs, or various other two CDR combinations, could be used as a therapeutic product. Alternatively, these clones could be converted to framework germline sequences (either with or without codon optimization) for use as a therapeutic agent so long as activity was maintained.

### Example 4

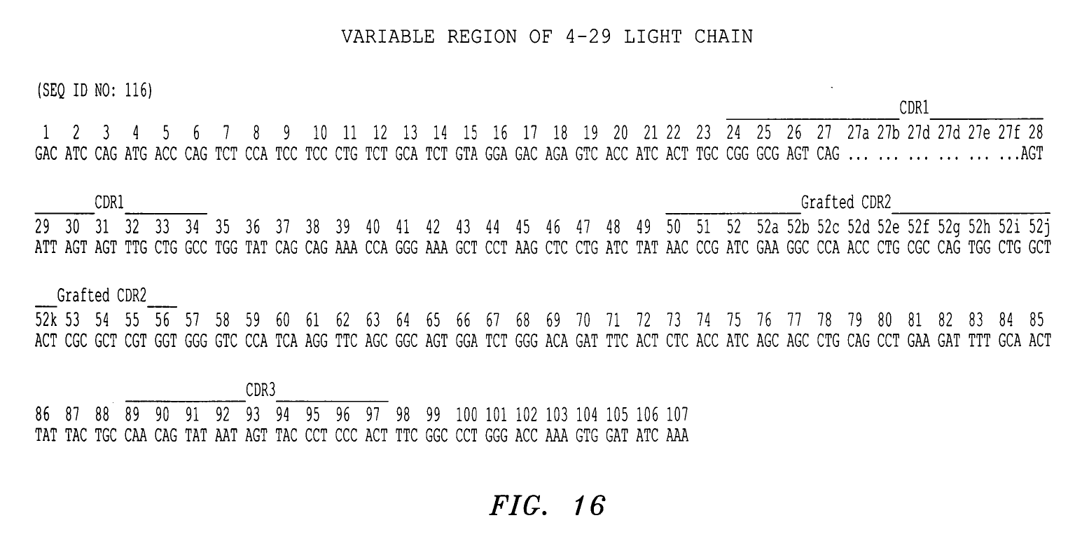

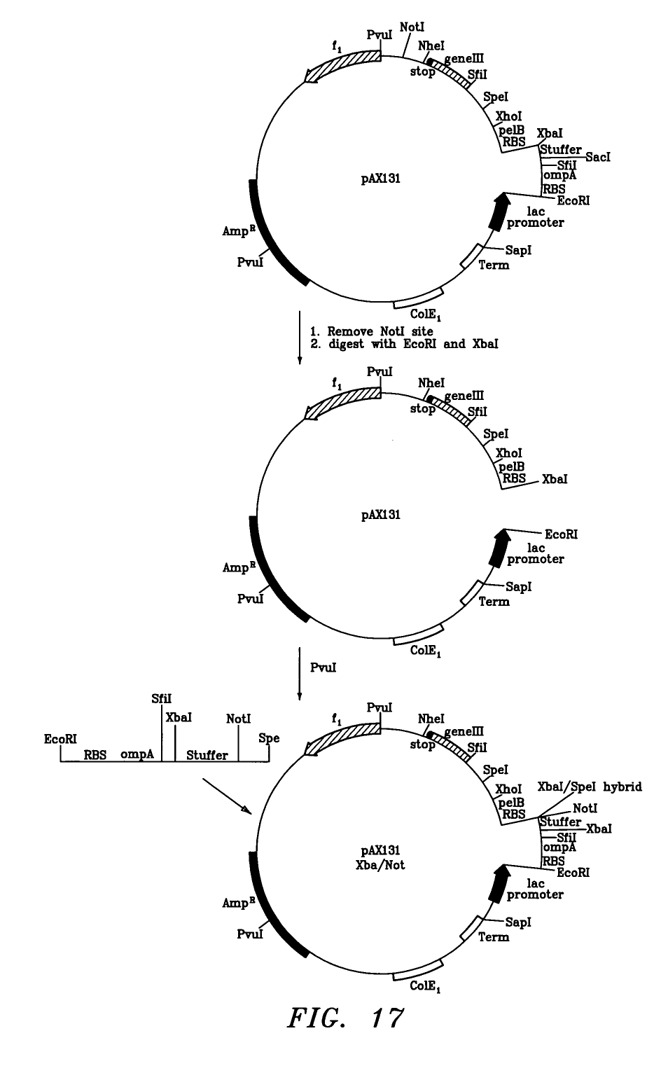

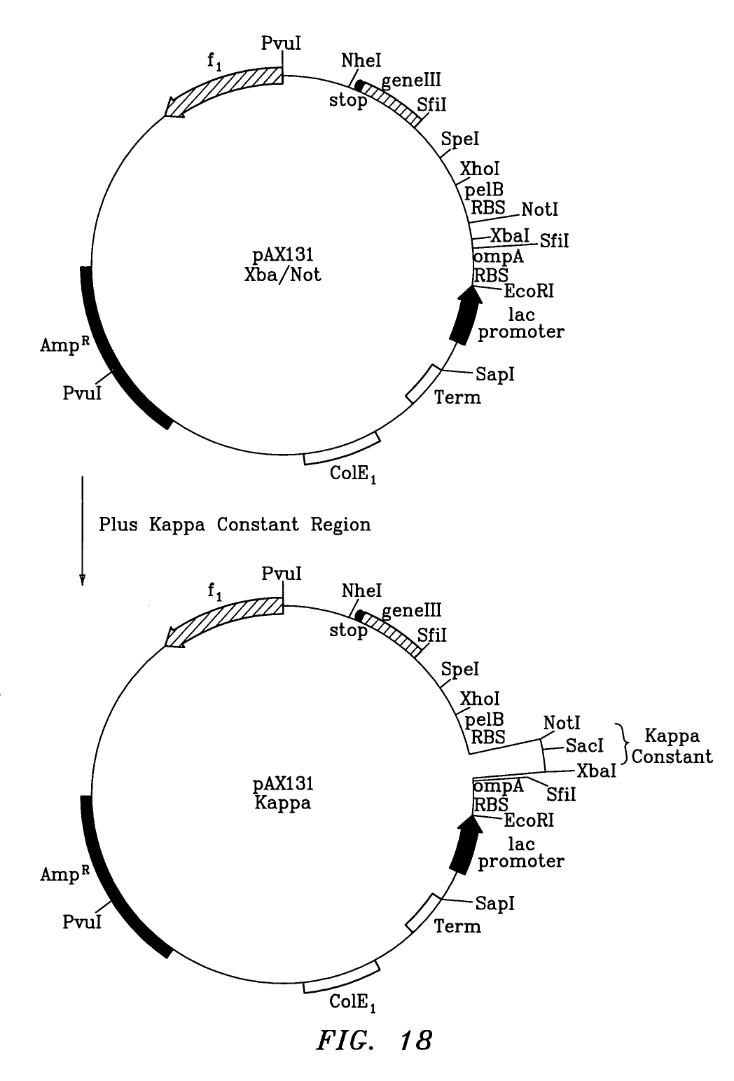

The TPO mimetic peptide graft in Fab clone X4b has been transplanted into the heavy chain CDR3 of another antibody framework, 5G1.1. Construction of 5G1.1 is described in U.S. Appln. Ser. No. 08/487,283, incorporated herein by reference. The sequence was cloned into 5G1.1 in such a fashion as to replace the native CDR3 with 5′ ttg cca ATT GAA GGG CCG ACG CTG CGG CAA TGG CTG GCG GCG CGC GCG cct gtt 3′ (SEQ. ID. NO: 65). The peptide graft translated into amino acids is Leu Pro Ile Glu Gly Pro Thr Leu Arg Gln Trp Leu Ala Ala Arg Ala Pro Val (SEQ. ID. NO: 66). The 5G1+ peptide was produced as a whole IgG antibody (See FIGS. 13A and 13B).

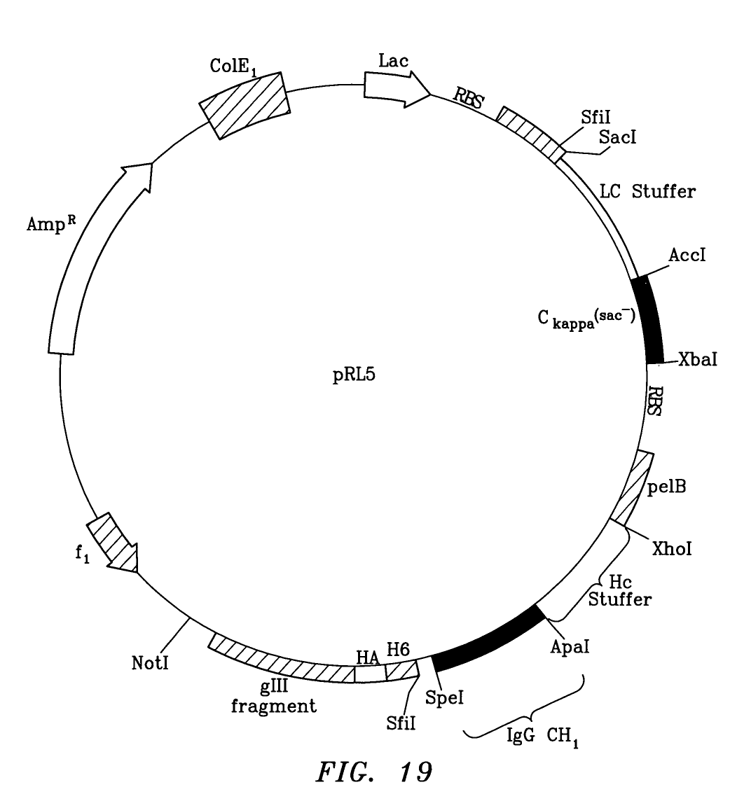

Purified 5G1.1+ peptide antibody as well as the parental 5G1.1 were analyzed for their ability to bind to cMpl receptor by FACS analysis. Binding to receptor expressing and non-receptor expressing 293 cells was compared. See FIG. 14. The FACS staining was performed essentially as described previously herein, with the exception that the detection was done using PE conjugated F(ab′)2 fragment of goat anti-human IgG (H+L). The negative controls of 3° only anti-tetanus toxoid irrelevant Fab, and Fab X1a which binds weakly to cMpl receptor all showed very little staining. However, binding Fabs X1c and X4b showed strong staining as did the 5G1.1+ peptide. None of those clones demonstrated binding to the non-receptor expressing cells indicating that the cell staining is occurring through specific recognition of the cMpl receptor. The parental 5G1.1 without the TPO mimetic peptide did not show staining to any of the cells tested.

The ability of the 5G1.1+ peptide whole IgG to activate the cMpl receptor using the luciferase reporter assay has been determined (see FIG. 15). The results herein indicate that the configuration of a whole IgG causes steric limitations in its ability to productively bring the two cMpl receptors together for activation. The activity of the 5G1.1 full IgG construct containing the TPO mimetic peptide in the heavy chain CDR3 positions, was only weakly activating and required approximately 100-200 fold higher molar concentrations as compared to TPO, to stimulate equivalent activity. As was previously observed with the binding experiments, activation by the 5G1.1 containing the peptide was observed only when the cMpl-R was expressed on the cell surface. No receptor specific binding or activity was observed with the parental 5G1.1 not containing the peptide. These results demonstrate that binding and activity of the TPO mimetic peptide and selected amino acid flanking sequences is not limited to or specific for the Tetanus Toxoid antibody framework, but can be applied to other antibody frameworks. Thus the flanking amino acid sequences that were selected during panning are specific for presentation of the TPO mimetic peptide within a given CDR position, but not for amino acid sequence of the antibody framework.

### Example 5

Library Construction Of EpO Mimetic Sequences Grafted Into A Human Antibody Framework

An agonist EPO mimetic-peptide DYHCRMGPLTWVCKPLGG (SEQ. ID. NO: 3) (designated EMP2 in Wrighton et al. 1996) was grafted separately into the anti-tetanus toxoid Fab heavy and light chain CDR3 region creating two antibody libraries as XXDYHXRMGPLTWVXKPLGGXX (SEQ. ID. NO: 71). Randomized positions were generated using an NNK doping strategy. As with the TPO mimetic peptide, two amino acids flanking the EPO mimetic peptide were randomized in order to select for the optimum presentation of the peptide. In addition the cysteine residues, which formed a disulfide bridge in the original cyclic peptide, were randomized. This was done not only because the CDRs already form loop structures and so the disulfide bridge was not necessary to constrain the peptide, but also because the cysteines might in fact disrupt the normal disulfide bonds of the antibody.

The CDR 3 of the anti-TT antibody heavy chain was completely replaced by the EPO peptide library graft. The generation of the library was essentially as described for the TPO heavy chain CDR2 library. Two alternate primers were used for the HCDR3 library: the reverse primer HR3 EPO ANTI (5′ CAC CCA GGT CAG TGG GCC CAT GCG MNN ATG ATA GTC MNN MNN TCT CGC ACA ATA ATA TAT GGC 3′)(SEQ. ID. NO: 21) that annealed at the end of the heavy chain FR3, and forward primer HR3 EPO CODE (5′ CGC ATG GGC CCA CTG ACC TGG GTG NNK AAA CCA CTG NNK NNK TGG GGC CAA GGG ACC ACG GTC 3′)(SEQ. ID. NO: 22) which annealed at FR4 of the heavy chain.

The light chain CDR3 EPO peptide library was constructed essentially as described above for the light chain CDR3 TPO peptide library using reverse primer LR3 EPO ANTI (5′ CAC CCA GGT CAG TGG GCC CAT GCG MNN ATG ATA GTC MNN MNN ACA GTA GTA CAC TGC AAA ATC 3′)(SEQ. ID. NO: 23) that annealed at the end of light chain FR3 and forward primer LR3 EPO CODE (5′ CGC ATG GGC CCA CTG ACC TGG GTG NNK AAA CCA CTG NNK NNK TTC GGC CM GGG ACC MG GTG 3′)(SEQ. ID. NO: 24) which annealed to FR4 of the light chain.

Selection Of The Epo Mimetic Peptide Heavy Chain Cdr3 Library And Light Chain Cdr3 Library

Selection for peptide presentation was performed by solid phase panning on the soluble EPO receptor. In this method, 1 ug of purified human EPO-soluble R (hEPO-sR from R&D Systems, Minneapolis, Minn. cat#307-ER-050) was immobilized on a microtiter dish overnight at 4°. After washing off free hEPO-sR, the plates were blocked with 1% BSA/PBS for one hour at 37°. Phage, prepared as described above, were added to the wells and incubated two hours at 37°. Washes were performed using PBS/0.5% Tween 20 for 5′ at room temperature per wash. 1, 5, 10, and 10 washes were performed in the first, second, third and fourth panning rounds respectively. After the washing steps were completed, the bound Fab-phage were eluted with 30 uls of elution buffer for 10′ at room temperature. Eluted phage were then neutralized and amplified as described in Example 1.

### Example 6

A library was generated by the insertion of a TPO mimetic peptide and previously selected flanking amino acids (NP-IEGPTLRQWLAARA-RG) (SEQ. ID. NO: 61) into a collection of human kappa gene fragments, in this case the CDR2 of the light chain. Stocks of human kappa light chains from multiple human peripheral blood lymphocyte (PBL) donors had been previously generated and cloned into pBluescript II SK+. Those constructs served as the source of antibody gene fragments.

Antibody Gene Banks

Total RNA from human PBLs was isolated using TRI Reagent (Molecular Research Center, Cincinnati, Ohio.) followed by mRNA purification with Oligotex mRNA purification System (QIAGEN, Valencia, Calif.) according to kit instructions. First strand cDNA was made using SuperScript RTase II cDNA Synthesis Kit (Life Technologies, Rockville, Md.) with a modified oligo dT primer. The sequence of the primer was 5′ TAGGATGCGGCCGCACAGGTC(T₂₀) 3′(SEQ. ID. NO: 62). Samples were cleaned up over a PCR purification Kit spin column (QIAGEN, Valencia, Calif.) according to kit directions. Light chain products were amplified using the reverse “Not I” primer and forward primers which annealed at the framework 1 (FR1) position of Kappa chains on the 1^{st} strand cDNA. The “Not I” primer had sequence which was identical to the 5′ end of the modified oligo dT primer (5′ TAGGATGCGGCCGCACAGGTC 3′)(SEQ. ID. NO: 72). The set of Kappa FR1 primers used were:

| | |
|---|---|
| XVB Vk1a | |
| CACGCGCACAACACGTCTAGA | (SEQ. ID. NO: 73) |
| RACATCCAGATGACCCAG | |
| XVB Vk1b | |
| CACGCGCACAACACGTCTAGA | (SEQ. ID. NO: 74) |
| GMCATCCAGTTGACCCAG | |
| XVB Vk1c | |
| CACGCGCACAACACGTCTAGA | (SEQ. ID. NO: 75) |
| GCCATCCRGATGACCCAG | |
| XVB Vk1d | |
| CACGCGCACAACACGTCTAGA | (SEQ. ID. NO: 76) |
| GTCATCTGGATGACCCAG | |
| XVB Vk2a | |
| CACGCGCACAACACGTCTAGA | (SEQ. ID. NO: 77) |
| GATATTGTGATGACCCAG | |
| XVB Vk2b | |
| CACGCGCACAACACGTCTAGA | (SEQ. ID. NO: 78) |
| GATRTTGTGATGACTCAG | |
| XVB Vk3a | |
| CACGCGCACAACACGTCTAGA | (SEQ. ID. NO: 79) |
| GAAATTGTGTTGACRCAG | |
| XVB Vk3b | |
| CACGCGCACAACACGTCTAGA | (SEQ. ID. NO: 80) |
| GAAATAGTGATGACGCAG | |
| XVB Vk3c | |
| CACGCGCACAACACGTCTAGA | (SEQ. ID. NO: 81) |
| GAAATTGTAATGACACAG | |
| XVB Vk4a | |
| CACGCGCACAACACGTCTAGA | (SEQ. ID. NO: 82) |
| GACATCGTGATGACCCAG | |
| XVB Vk5a | |
| CACGCGCACAACACGTCTAGA | (SEQ. ID. NO: 83) |
| GAAACGACACTCACGCAG | |
| XVB Vk6a | |
| CACGCGCACAACACGTCTAGA | (SEQ. ID. NO: 84) |
| GAAATTGTGCTGACTCAG | |
| XVB Vk6b | |
| CACGCGCACAACACGTCTAGA | (SEQ. ID. NO: 85) |
| GATGTTGTGATGACACAG | |

A typical amplification reaction contained 2 μls cDNA reaction, dNTPs, “Not I” reverse primer, one of the XVB forward primers, Opti-prime buffer #5 (Stratagene, La Jolla, Calif.), and Expand High Fidelity polymerase mixture (Roche Molecular Biochemicals, Indianapolis, Ind.). Samples were heated to 94° C. for 2 minutes, then carried through 10 cycles of 94° C. for 15 seconds, 56° C. for 30 seconds, and 72° C. for 1 minute, followed by 20 cycles of 94° C. for 15 seconds, 56° C. for 30 seconds, and 72° C. for (1 minute+5 seconds/cycle). The cycles were followed by an extended incubation at 72° C. (7 minutes) prior to 4° C. hold. Products were ethanol precipitated and then gel purified. Fragments of approximately 850bp were isolated and then digested with Xba I and Sac I. The resulting kappa products were ligated into pBluescript II SK+ that had likewise been digested with Xba I and Sac I. The ligation products were electroporated into Top10F′ (Invitrogen, Carlsbad, Calif.) and grown overnight. The bacterial pellet was used to isolate the Kappa library DNA with QIAGEN's MAXlprep DNA isolation kit.

Construction Of Framework Library With TPO Mimetic Peptide

For construction of the TPO light chain framework library, equal amounts of four different kappa light chain libraries from four different patients were used as the starting template for the PCR reactions (25ng total per reaction). The TPO mimetic peptide and selected flanking amino acids were incorporated into the light chains by overlap PCR. In the first round of PCR a set of reverse primers (VK ANTI primers) which bound to the kappa light chain FR2 were separately combined with the forward T7 seq-F primer (5′-ATTMTACGACTCACTATAGGG-3′)(SEQ. ID. NO: 86) to synthesize the N terminal piece of the light chain and part of the TPO mimetic peptide within the LC CDR2 position. A second set of forward primers (VK CODE primers), which bound to FR3, were combined separately with the T3 reverse primer (5′-AATTAACCCTCACTAAAGGG-3′) (SEQ. ID. NO: 87) to synthesize the rest of the TPO mimetic peptide within the LC CDR2 position and the C terminal half of the light chain by PCR. Separate reactions were performed for each pair of primer combinations in duplicate.

| | |
|---|---|
| VK6ANTI | |
| 5′AGCCAGCCACTGGCGCAGGGTTGGGCCTTC | (SEQ. ID. NO: 88) |
| GATCGGGTTCCTGATGAGGAGCTTTGGRG-3′ | |
| VK5ANTI | |
| 5′AGCCAGCCACTGGCGCAGGGTTGGGCCTTC | (SEQ. ID. NO: 89) |
| GATCGGGTTTTGAATAATGAAAATAGCAG-3′ | |
| VK4ANTI | |
| 5′AGCCAGCCACTGGCGCAGGGTTGGGCCTTC | (SEQ. ID. NO: 90) |
| GATCGGGTTGTAAATGAGCARCTTAGGAG-3′ | |
| VK3ANTI | |
| 5′AGCCAGCCACTGGCGCAGGGTTGGGCCTTC | (SEQ. ID. NO: 91) |
| GATCGGGTTATAGATGAGGAGCCTGGGMG-3′ | |
| VK2AANTI | |
| 5′AGCCAGCCACTGGCGCAGGGTTGGGCCTTC | (SEQ. ID. NO: 92) |
| GATCGGGTTATAAATTAGGCGCCTTGGAG-3′ | |
| VK2BANTI | |
| 5′AGCCAGCCACTGGCGCAGGGTTGGGCCTTC | (SEQ. ID. NO: 93) |
| GATCGGGTTATAGATYAGGAGCTGTGGAG-3′ | |
| VK1AANTI | |
| 5′AGCCAGCCACTGGCGCAGGGTTGGGCCTTC | (SEQ. ID. NO: 94) |
| GATCGGGTTATAGATCAGGAGCTTAGGA-3′ | |
| VK1BANTI | |
| 5′AGCCAGCCACTGGCGCAGGGTTGGGCCTTC | (SEQ. ID. NO: 95) |
| GATCGGGTTRTAGATCAGGAGCTTAGGG-3′ | |
| VK1CANTI | |
| 5′AGCCAGCCACTGGCGCAGGGTTGGGCCTTC | (SEQ. ID. NO: 96) |
| GATCGGGTTATAGATCAGGGACTTAGGG-3′ | |
| VK1DANTI | |
| 5′AGCCAGCCACTGGCGCAGGGTTGGGCCTTC | (SEQ. ID. NO: 97) |
| GATCGGGTTATAGATCAGGYGCTTAGGG-3′ | |
| VK6CODE | |
| 5′CCAACCCTGCGCCAGTGGCTGGCTGCTCGC | (SEQ. ID. NO: 98) |
| GCTCGTGGTGGGGTCCCCTCGAGGTTCAG-3′ | |
| VK5CODE | |
| 5′CCAACCCTGCGCCAGTGGCTGGCTGCTCGC | (SEQ. ID. NO: 99) |
| GCTCGTGGTGGAATCCCACCTCGATTCAG-3′ | |
| VK4CODE | |
| 5′CCAACCCTGCGCCAGTGGCTGGCTGCTCGC | (SEQ. ID. NO: 100) |
| GCTCGTGGTGGGGTCCCTGACCGATTCAG-3′ | |
| VK3ACODE | |
| 5′CCAACCCTGCGCCAGTGGCTGGCTGCTCGC | (SEQ. ID. NO: 101) |
| GCTCGTGGTGGCATCCCAGMCAGGTTCAG-3′ | |
| VK3BCODE | |
| 5′CCAACCCTGCGCCAGTGGCTGGCTGCTCGC | (SEQ. ID. NO: 102) |
| GCTCGTGGTGGTATCCCAGCCAGGTTCAG-3′ | |
| VK2ACODE | |
| 5′CCAACCCTGCGCCAGTGGCTGGCTGCTCGC | (SEQ. ID. NO: 103) |
| GCTCGTGGTGGAGTSCCAGAYAGGTTCAG-3′ | |
| VK2BCODE | |
| 5′CCAACCCTGCGCCAGTGGCTGGCTGCTCGC | (SEQ. ID. NO: 104) |
| GCTCGTGGTGGGGTCCCWGACAGRTTCAG-3′ | |
| VK1ACODE | |
| 5′CCAACCCTGCGCCAGTGGCTGGCTGCTCGC | (SEQ. ID. NO: 105) |
| GCTCGTGGTGGGGTCCCATCAAGGTTCAG-3′ | |
| VK1BCODE | |
| 5′CCAACCCTGCGCCAGTGGCTGGCTGCTCGC | (SEQ. ID. NO: 106) |
| GCTCGTGGTGGGGTCCCATCTCGGTTCAG-3′ | |

Fragments from the first rounds of PCR were gel purified. Those purified fragments were then combined, in an antibody family specific manner, in overlap PCR reactions to generate the full light chain. Reactions for each family were performed in triplicate using 40 ng of both the N-terminal and C-terminal piece of the light chain in each reaction. The reactions were run for 10 cycles prior to the addition of the T3 and T7 Seq-F primers, followed by an additional 25 cycles after primer addition. The full length LC fusion PCR products were gel purified, digested with Sac I and Xba I, and then again gel purified. The light chain inserts were then ligated into an appropriate phage display vector, which had been similarly digested with Xba I and Sac I and gel purified. The pRL5-kappa vector used had restriction sites which were compatible with the LC fragments and contained the remaining Kappa constant region from the native Sac I site to the C-terminal Cys. In addition, the anti-tetanus toxoid heavy chain was inserted into the vector by Xho I and Spe I for Fab production.

The ligation mixture was transformed by electroporation into XL-1 Blue bacteria (Stratagene, La Jolla Calif.) and amplified. The library was panned four rounds on 293 EBNA cells transfected with the cMpl-R in a manner similar to that previously described. Clones obtained during panning were screened for binding by FACs analysis on 293 EBNA cells transfected with or without the cMpl-R as previously described. A number of clones, which specifically bound the cMpl-R, were obtained. DNA fingerprinting of the resulting light chains by digestion with Bst N1 indicated that the clones could be divided into 5 different groups. Partial sequencing of 8 of these clones showed that frameworks from at least two different kappa light chain families were selected during the panning (VK1 and VK3). In an initial test, 3 of the light chain framework clones were combined with the heavy chain of X4b to create a Fab with 2 TPO mimetic peptides. These clones induced activation of the cMpl-R in luciferase assays as previously described. The level of activation using bacterial supernatants of one such clone 429/X4b (see FIG. 16 ) was approximately 10-20 fold lower than that observed with TPO, as estimated by comparing activity to known concentrations of TPO and using quantitative western blots to determine the concentration of the antibody in the supernatant. Additional clones can be screened in a similar fashion on order to identify clones with greater activity.

These Fabs, or various other LC, HC or intrachain CDR combinations, could be used as a therapeutic product. Alternatively, these clones could be converted to framework germline sequences (either with or without codon optimization) for use as a therapeutic agent so long as activity was maintained.

Modification of the Phage Display Vectors PAX131 and pRL5

The Not I site in pAX131 was removed by digesting the vector with Not I, using Klenow polymerase to fill in the Not I overhangs, and then re-ligating the vector. See FIG. 17. Further modification was made by digesting pAX 131 with EcoR I and Xba I. An insert that replaced the elements removed by such digestion was generated using overlapping oligonucleotides with the following changes: conversion of the Sac I site to a new Xba I site (single underline in primer sequences below), conversion of the original Xba I site to a Not I site (double underline in the primer sequences below), and ending the insert with a Spe I overhang which is compatible with the vector's Xba I digest generated overhang. Ligation of the EcoR I/Spe I insert into the EcoR I/Xba I cut vector resulted in an Spe I/Xba I hybrid which will no longer cut with either Spe I or Xba I at that site. The sequence of the oligos used.were: “EcoSpe” 5′ AA TTC AAG GAG TTA ATT ATG AAA AAA ACC GCG ATT GCG ATT GCG GTG GCG CTG GCG GGC TTT GCG ACC GTG GCC CAG GCG GCC TCT AGA ATC TGC GGC CGC a 3′(SEQ. ID. NO: 107), and “SpeEco” 5′ ct agt GCG GCC GCA GAT TCT AGA GGC CGC CTG GGC CAC GGT CGC AAA GCC CGC CAG CGC CAC CGC AAT CGC AAT CGC GGT TTT TTT CAT AAT TAA CTC CTT G 3′ (SEQ. ID. NO: 108).

The intermediate vector created was pAX131Xba/Not. The human kappa constant region was inserted between the Xba I and Not I sites generating pAX131-kappa (see FIG. 18). The human kappa constant region was PCR amplified from human cDNA using primers that introduced the upstream Xba I site and in the downstream position a TAA stop codon followed by a Not I site. The primers used were CKXba I (5′ GGA GTC TAG ATA ACT GTG GCT GCA CCA TCT GTC TTC 3′) (SEQ. ID. NO: 109) and CKNotI (5′ AGG AGC GGC CGC TTA ACA CTC TCC CCT GTT GAA GCT C 3′)(SEQ. ID. NO: 110).

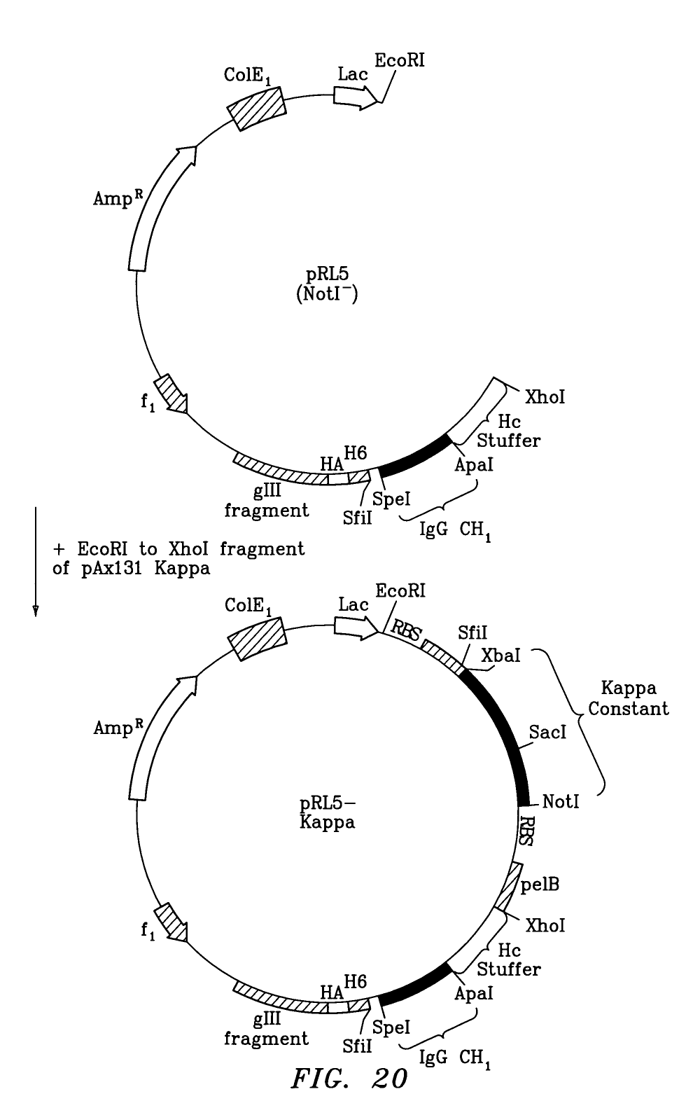

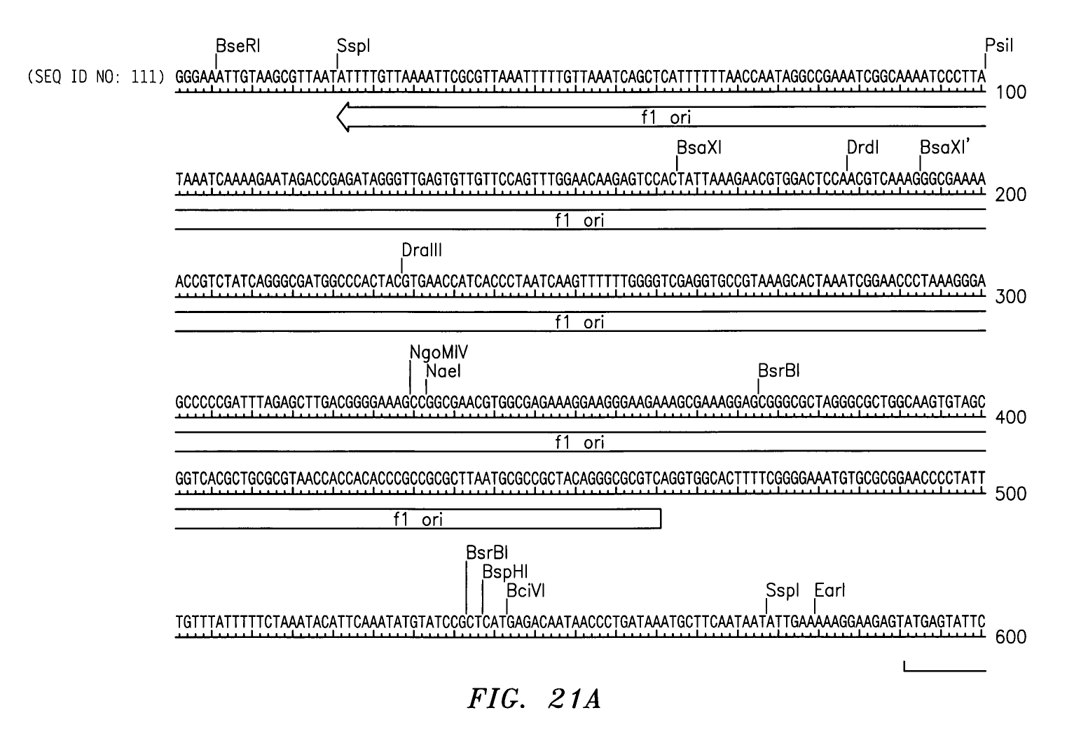

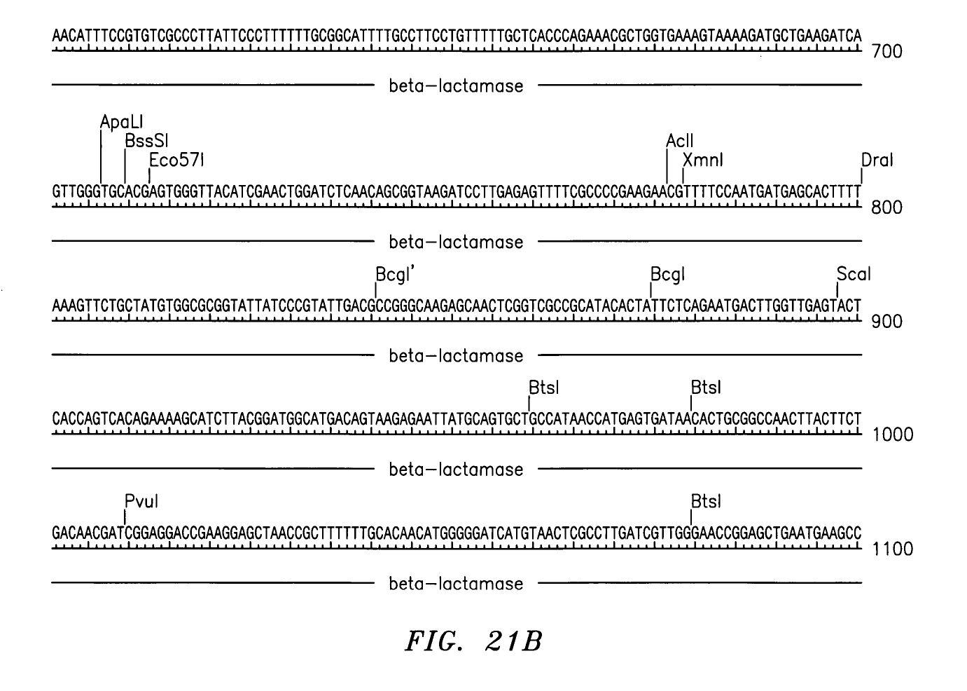

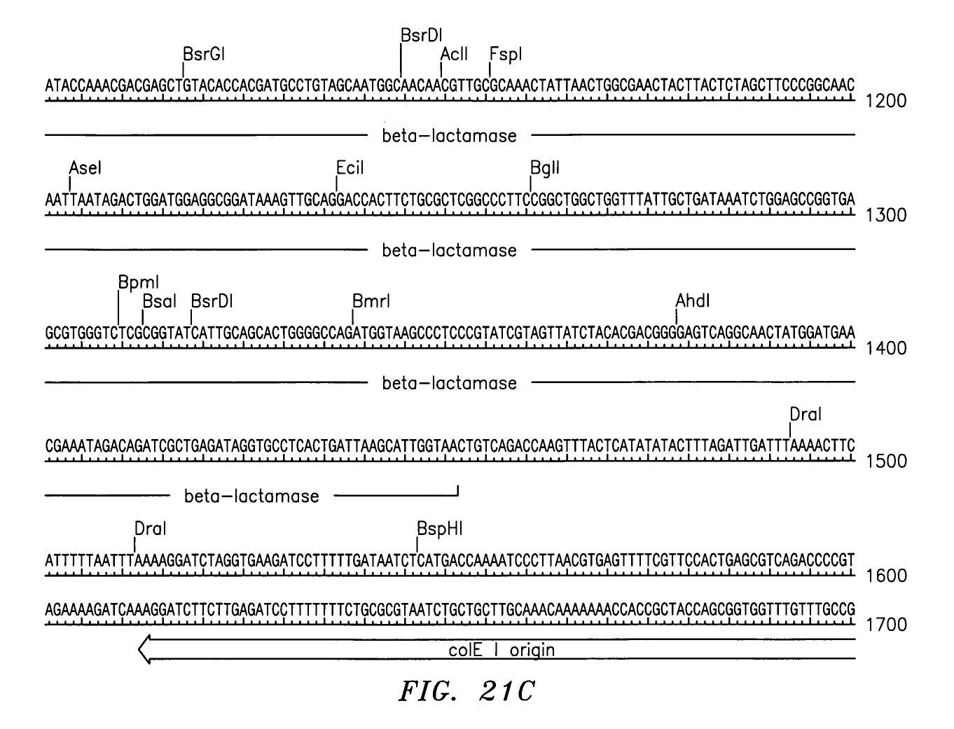

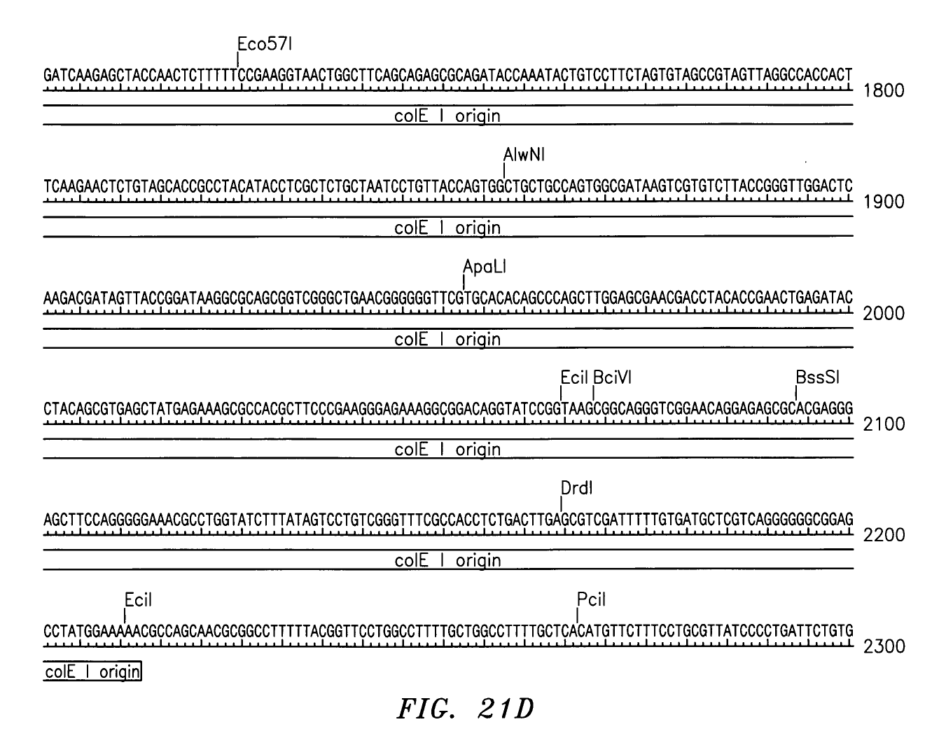

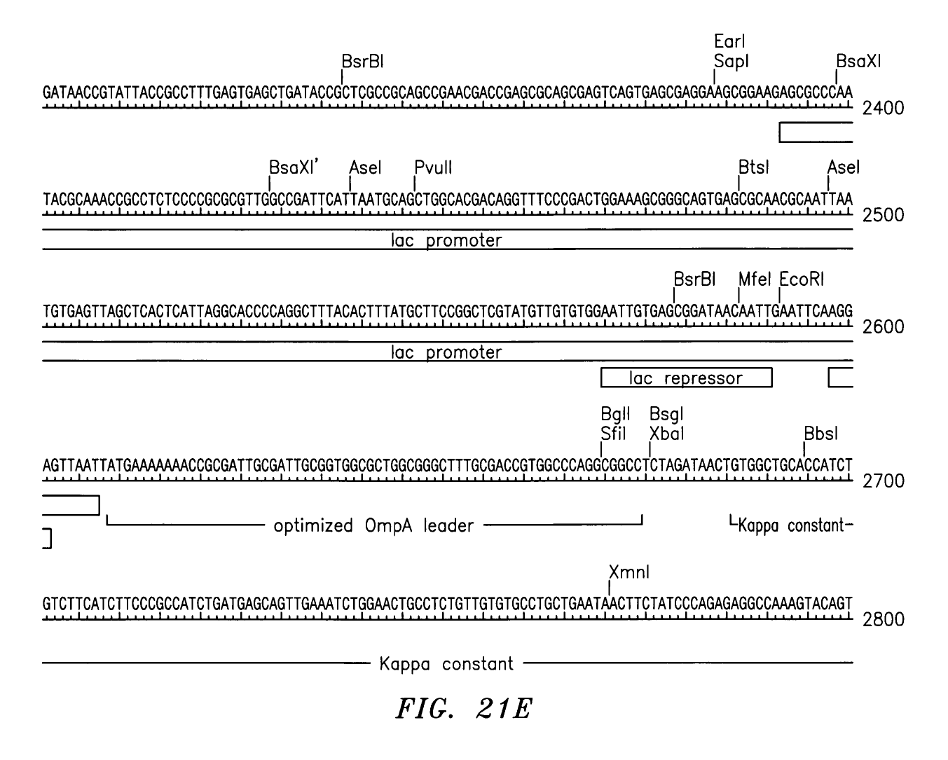

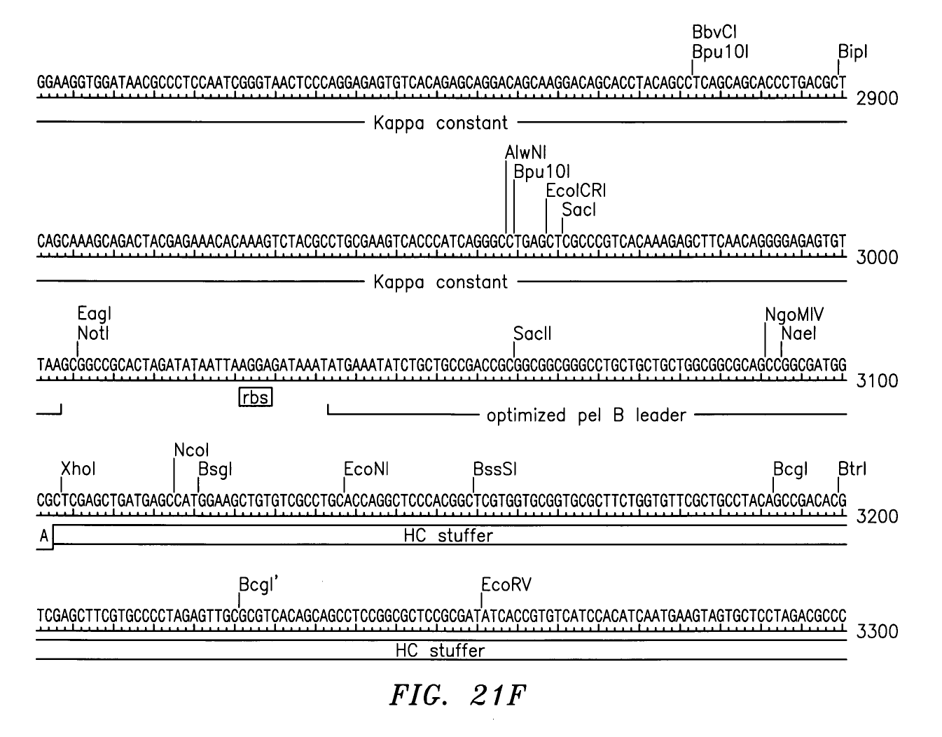

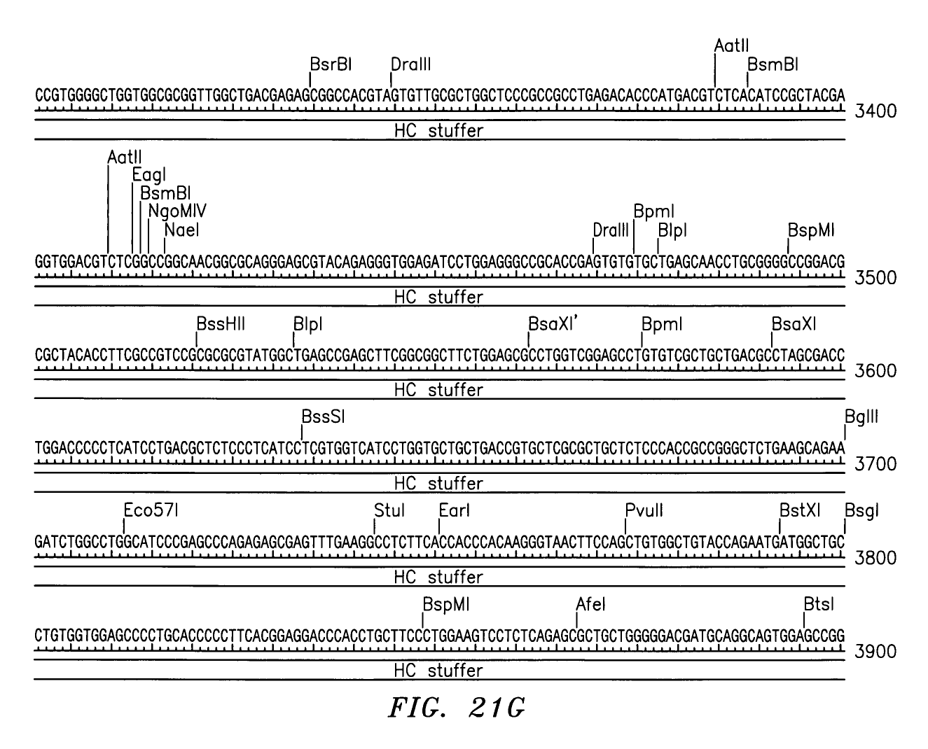

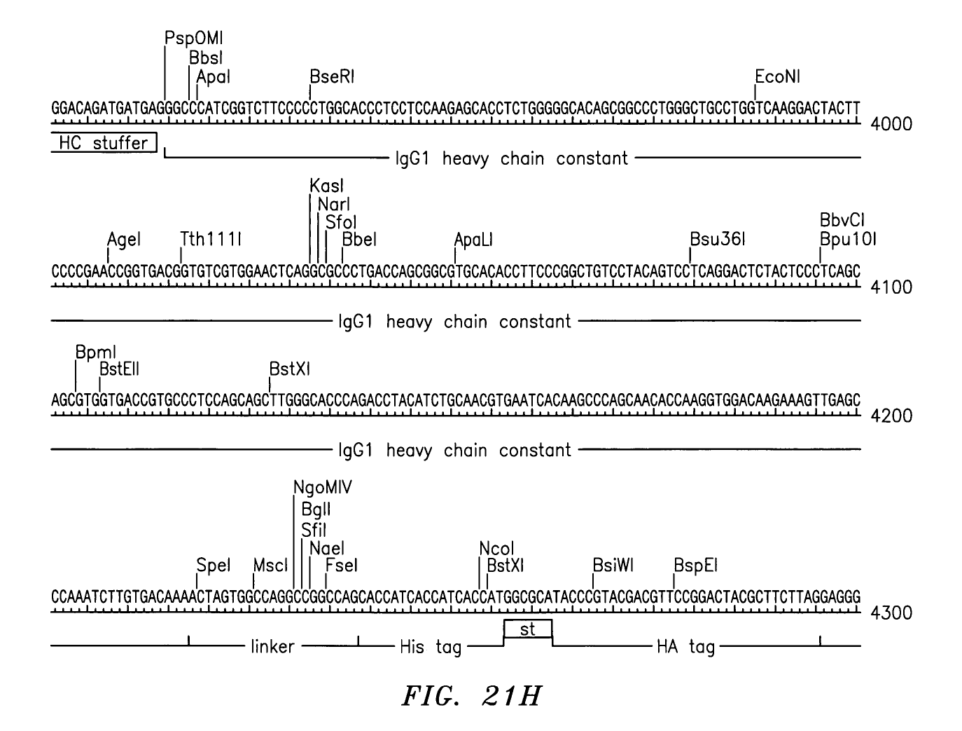

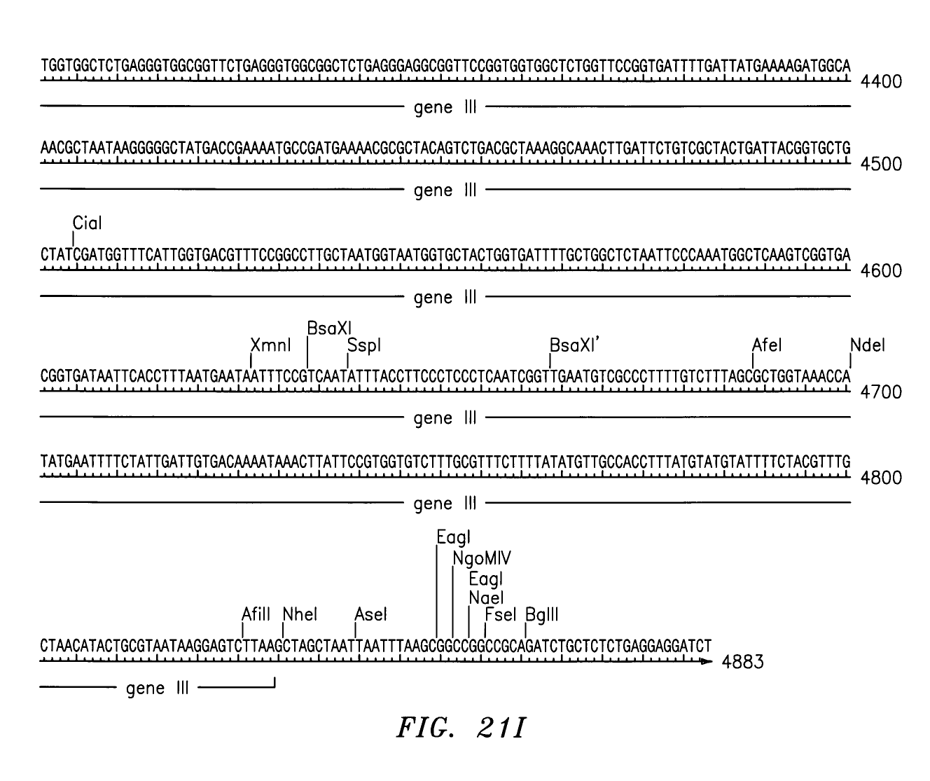

The light chain cloning modifications incorporated into pAX131-kappa were shuttled into the pRL5 vector (modified to have its Not I site removed as described above) by moving the EcoR I to Xho I fragment. See FIGS. 19 and 20. This vector was designated as pRL5-kappa. (See FIGS. 21A-I.)

### Example 7

### (Core H-CDR2 Library)

An additional HC CDR2 library was constructed where a core sequence of the TPO mimetic peptide (GPTLRQWL) (SEQ. ID. NO: 112) flanked by a single random amino acid on each side was inserted into the heavy chain partially replacing the CDR2 (GXGPTLRQWLXYAQKFQG) (SEQ. ID. NO: 113). The construction of the heavy chain partial CDR2 replacement library was also carried out as previously described for the heavy chain CDR2 library with the exception that the specific primer 8HCR2anti0

(5′ CAGCCACTGGCGCAGGGTTGGGCCMNNCCCTCCCATCCACTCMGCCC-3′) (SEQ. ID. NO: 114) which annealed at the end of the heavy chain FR2 and the primer 8HCR2code(5′GGCCCMCCCTGCGCCAGTGGCTGNNKTACGCACAGAAATTCCAG GGCAGAGTCACCATT-3′)(SEQ. ID. NO: 115) which annealed to the beginning of the heavy chain FR3 were used in place of the previously described HR2 cMpl primers. The heavy chain partial CDR2 replacement library was panned four rounds on 293 EBNA cells transfected with the cMpl-R as described previously. These clones can now be screened for positive binding clones by FACs analysis as previously described.

### Example 8

An individual clone was created by combining the heavy chain CDR3 clone X4b (See FIGS. 5 and 9), with the heavy chain CDR2 clone 19 (see FIG. 9) to create a new Fab clone containing two TPO mimetic peptides. This clone was further modified by codon optimization and conversion to germlne sequences. The sequence of the TT-TPO ab variable regions were broken into two sections where the Framework1 to CDR3 corresponds to the germline V region gene elements and Framework 4 to J regions. The V and J region sequences are compared separately to databases of the corresponding human germline elements. Blast searches were conducted at NCBI databank (http://www.ncbi.nlm.nih.gov/BLAST/)

The suitability of a germline V gene to act as the acceptor V region in a humanization procedure will be influenced by the likelihood of successful retaining antigen binding activity. In general, using germlne V gene as acceptor may not keep the affinity equal to rearranged one. In this case, two TPO peptides: “QLIEGPTLRQWLAARANS” (SEQ ID NO. 152) and “LPIEGPTLRQWLAARAPV” (SEQ ID NO. 39) were grafted into VH CDR2 and CDR3 respectively. These peptides provide the antigen binding activity of the pAXB116 antibody.

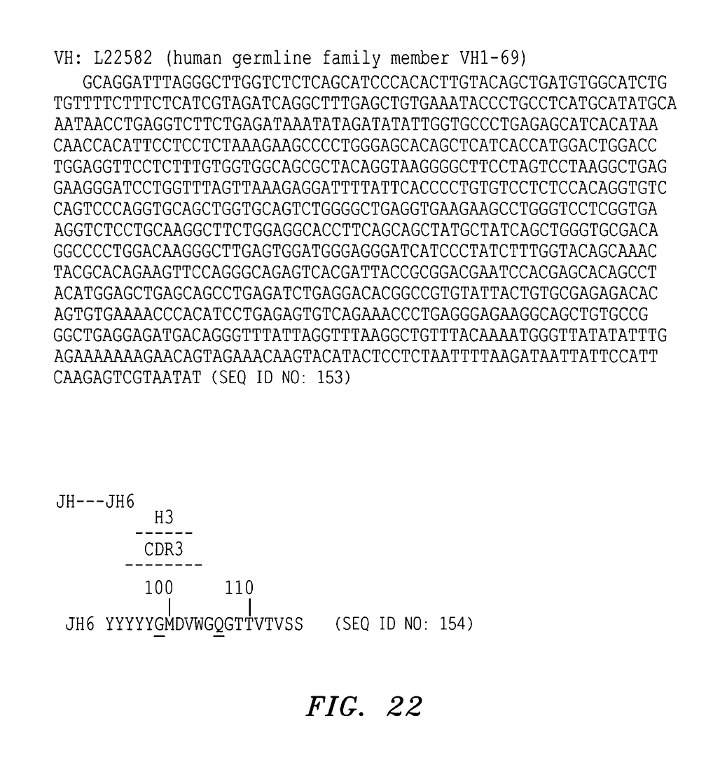

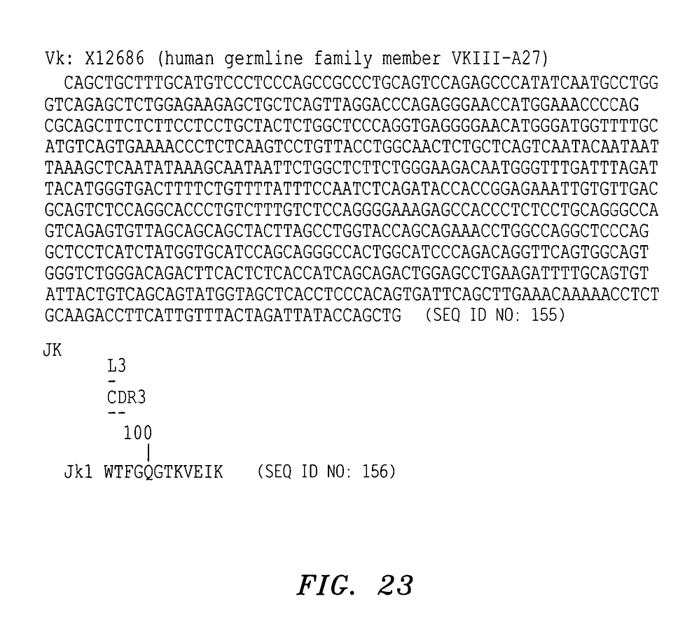

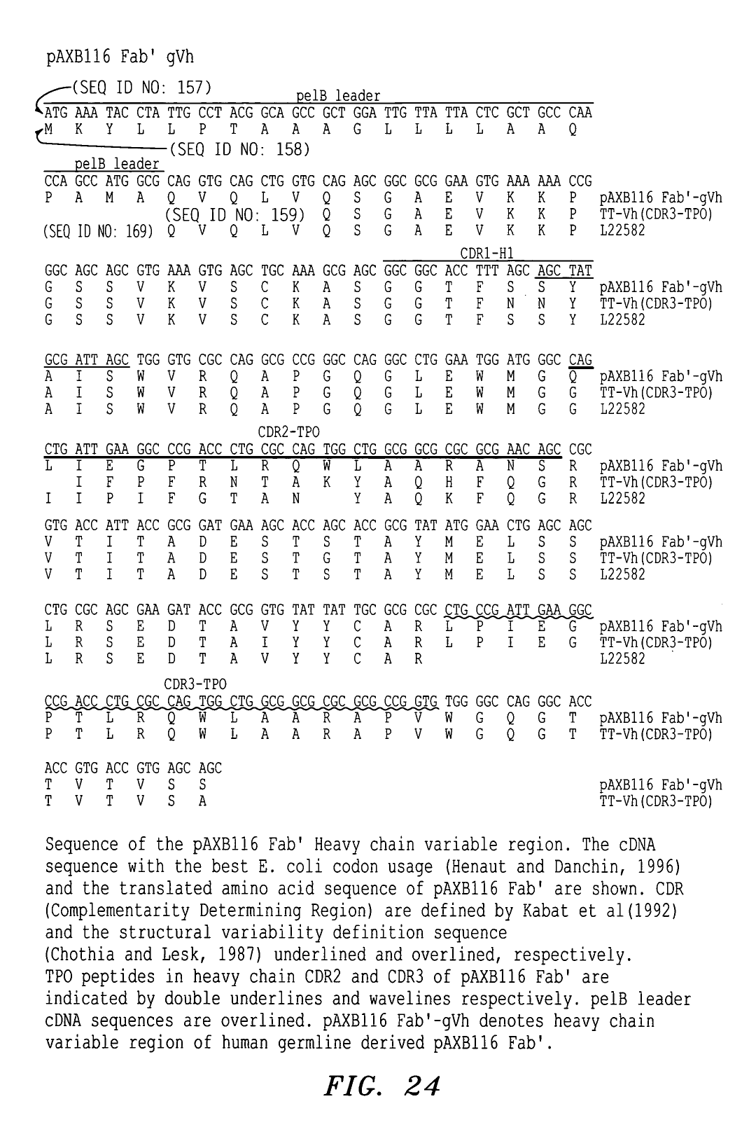

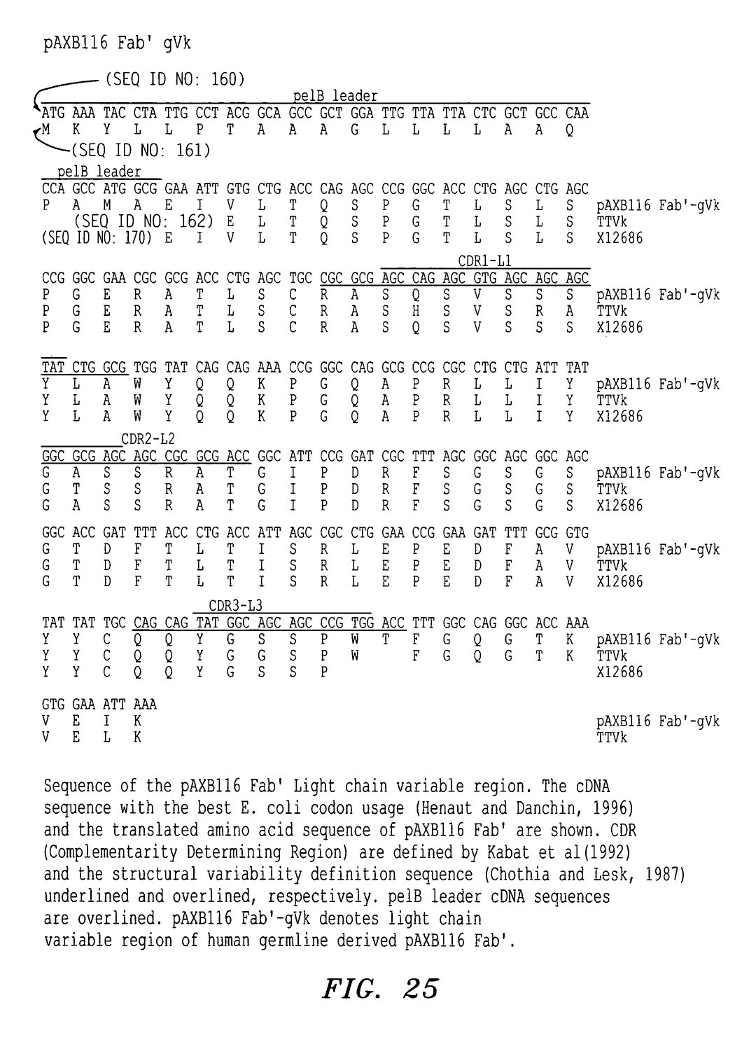

To minimize immunogenicity, the candidate acceptor V region should be conserved in the most of individuals and exhibit no allotypic variation. It should also be represented functionally in rearranged V region genes, so that the human population will be tolerant of the encoded polypeptide product. The highest homology of human germlne sequences with TT-TPO antibody variable regions are shown in FIGS. 22 and 23. FIGS. 24 and 25 show the sequence of the pAXB116 Fab′ Heavy and light chain variable regions, respectively. The cDNA sequence with the best E. coli codon usage (Henaut and Danchin: Analysis and Predictions from Escherichia coli sequences in: Escherichia coli and Salmonella, Vol. 2, Ch. 114:2047-2066,1996, Neidhardt FC ed., ASM press, Washington, D.C. http://www.biology.ualberta.ca/old_site/pilgrim.hp//links/usage2.0c.html) and the translated amino acid sequence of pAXB116 Fab′ are shown. CDR (Complementarity Determining Region) are defined by Kabat et al. (Kabat et al., (1992) Sequence of proteins of Immunological Interest. United States Department of Health and Human Service, Bethesda) and the structural variability definition sequence (Chothia C., and Lesk A. M. (1987), J. Mol. Biol. 196, 901-917) underlined and overlined, respectively. TPO peptides in heavy chain CDR2 and CDR3 of pAXB116 Fab′ are indicated by double underlines and wavelines respectively. pelB leader cDNA sequences are overlined . In FIG. 24, pAXB116 Fab′-gVk denotes light chain variable region of human germline derived pAxB116 Fab′. In FIG. 25, pAXB116 Fab′-gVh denotes heavy chain variable region of human germline derived pAXB116 Fab′.

Assembly of pAXB116 Construct

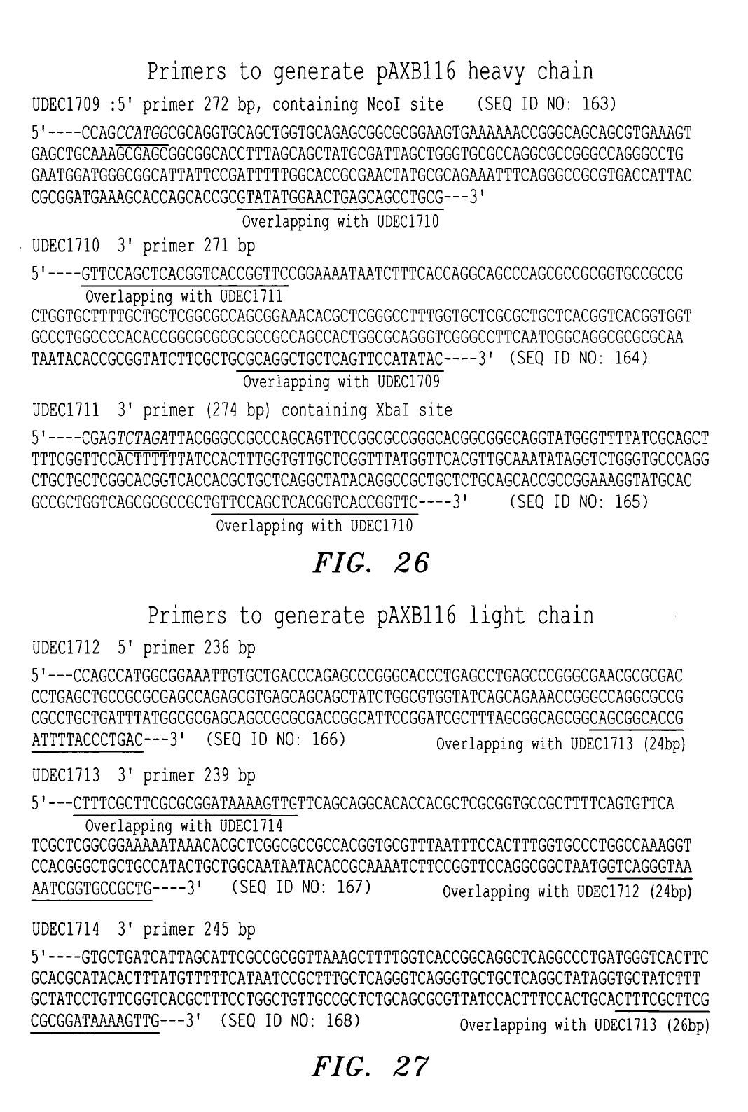

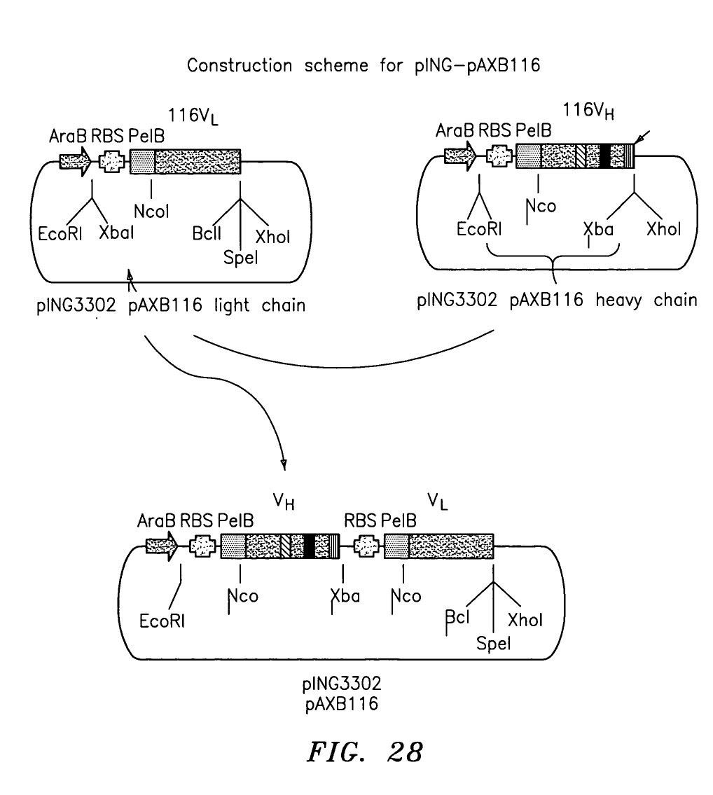

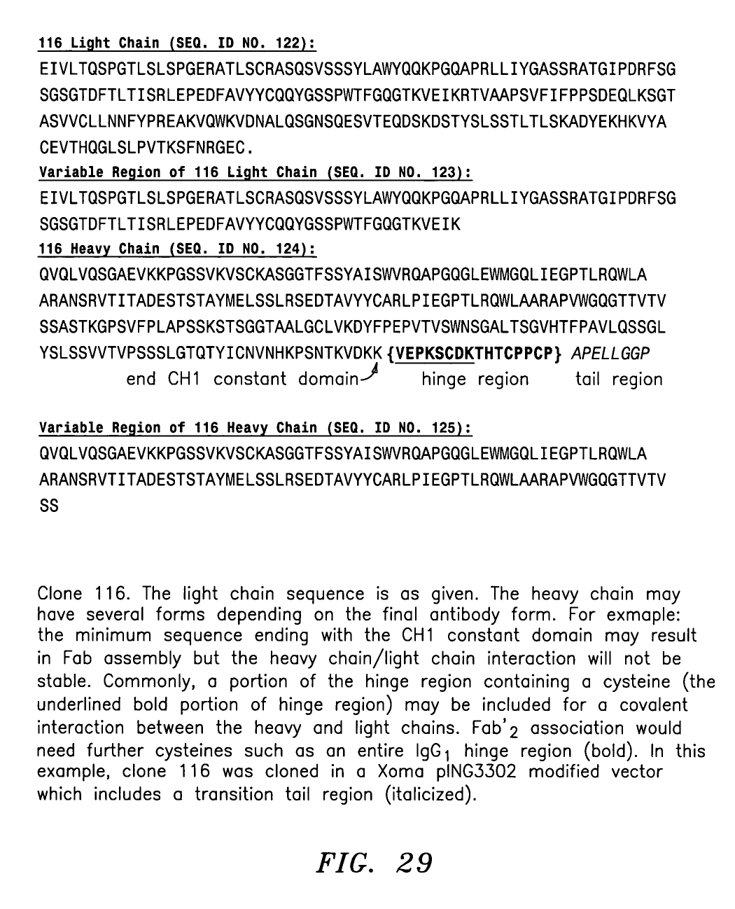

pAXB116 was generated synthetically using PCR technology. The pAXB116 variable heavy chain contained CDR2-TPO and CDR3-TPO was linked to the human IgG1 CH1+Hinge+2C9(hlgG1 CH2) (Better et al., Proc Natl Acad Sci U S A 1993 Jan 15;90(2):457-61) using three oligos (UDEC1709, UDEC1710 and UDEC1711 from Oligos Etc., Inc., see FIG. 26), the pAXB116 variable light chain was fused with human light chain constant region by three oligos ( UDEC1712, UDEC1713 and UDEC1714, see FIG. 27). The PCR products were TA cloned into pCR2.1-TOPO vector (TOPO TA cloning Kits, Invitrogen) and sequenced (MWG Biotech Inc.) confirmed. The pAXB116 heavy chain was digested with NcoI/XhaI and light chain by NcoI/XhoI. Fragments were gel isolated and separately cloned into pING3302 vector (commercially available from XOMA Inc., Berkeley, Calif., USA) with pelB leader and opened with NcoI/XhaI and NcoI/XhoI. The pING-pAXB116 Fab was generated by grafting heavy chain EcoRI/XhaI fragment into upstream of pING-pAXB116 light chain BcII/XhaI sites. FIG. 28 schematically shows the construction scheme for the pING-pAXB116 vector. The amino acid sequence of the pAXB116 heavy chain is shown in FIG. 29. The amino acid sequence of the pAXB116 light chain is shown in FIG. 29.

Expression of pAXB116 Fab′

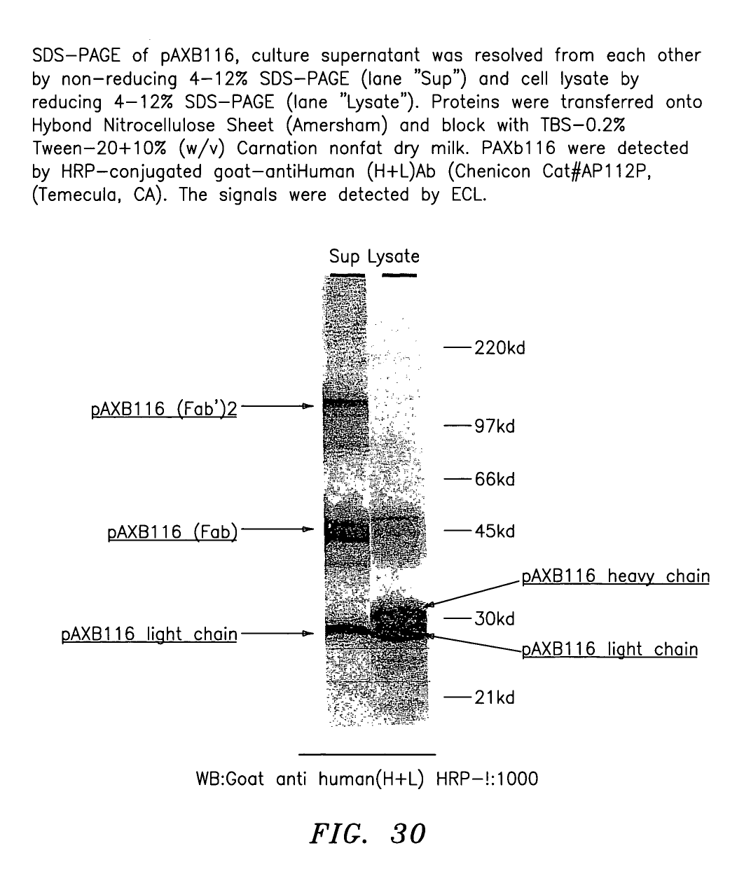

An Arabinose³¹ E.coli strain E104 (derivative of E. coli W3110 (ATCC 27352)) was transformed with pING-pAXB116 Fab construct and used for production of pAXB116 Fab′ and (Fab′)2. Transformants were grown at 37° C. in TYE medium (15 g Trypton, 10 g yeast Extract, 5 g NaCl per liter) supplemented with 15 ug/ml of tetracycline to about an OD600=0.4 . L-arabinose from 20% WN solution is added to a final concentration of 0.1%. The bacterial culture was then incubated for up to 16 hours post-induction at 37° C. Secreted products were detected directly in the cell-free culture supernatant. SDS-PAGE of pAXB116was conducted. Culture supernatant was resolved from each other by non-reducing 4-12% SDS-PAGE (lane “Sup”) and cell lysate by reducing 4-12% SDS-PAGE (lane “Lysate”). Proteins were transferred onto Hybond Nitrocellulose Sheet (Amersham) and blocked with TBS-0.2% Tween-20+10% (w/v) Carnation nonfat dry milk. PAXb116 were detected by HRP-conjugated goat-antiHuman (H+L)Ab (Chenicon Cat#AP112P, Temecula, Calif.). The signals were detected by ECL. The results are shown in FIG. 30.

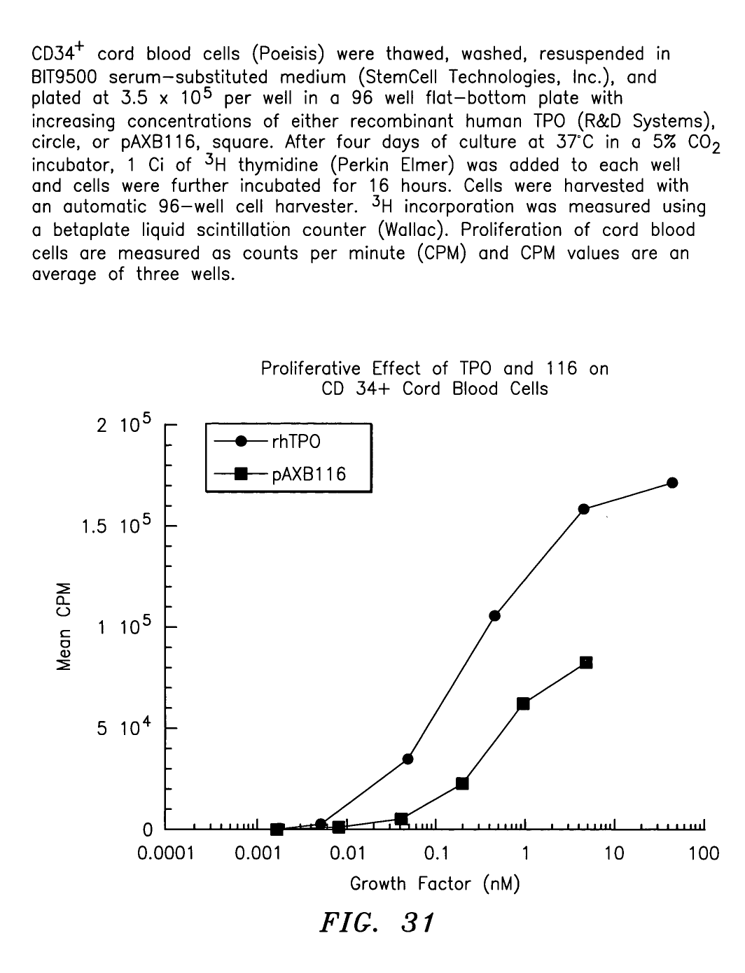

To show the proliferative effect of TPO on CD 34+Cord Blood Cells, CD34⁺ cord blood cells (Poeisis) were thawed, washed, resuspended in BIT9500 serum-substituted medium (StemCell Technologies, Inc.), and plated at 3.5×10⁵ per well in a 96 well flat-bottom plate with increasing concentrations of either recombinant human TPO (R&D Systems), circle, or pAXB116, square. After four days of culture at 37° C. in a 5% CO₂ incubator, 1 μCi of ³H thymidine (Perkin Elmer) was added to each well and cells were further incubated for 16 hours. Cells were harvested with an automatic 96-well cell harvester. ³H incorporation was measured using a betaplate liquid scintillation counter (Wallac). Proliferation of cord blood cells is measured as counts per minute (CPM) and CPM values are an average of three wells as shown in FIG. 31 .

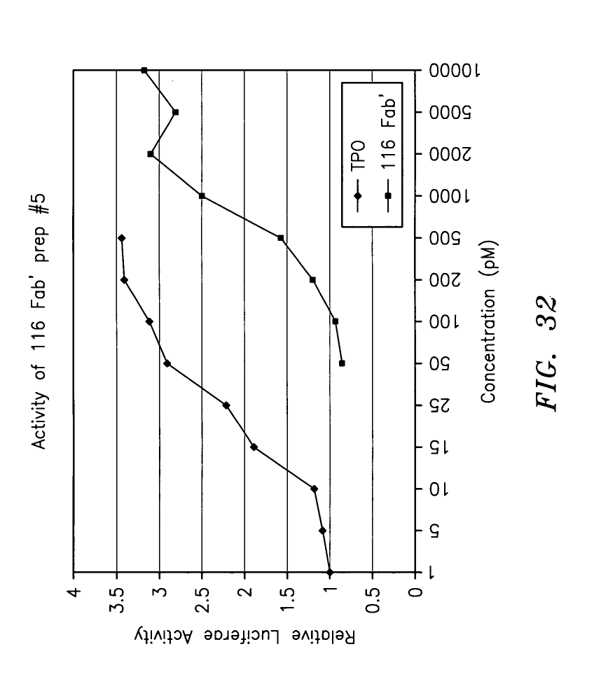

This new clone, which was named 116 (FIG. 29) was purified to 95% homogeneity. The specific activity of the purified 116 Fab was then tested using the luciferase based assays as previously described in Example 1. The specific activity of the purified 116 was found to be approximately 30 fold lower than TPO (FIG. 32).

### Example 9

A third heavy chain CDR2 library was constructed replacing part of the tetanus toxoid (TT) heavy chain CDR2 with the TPO mimetic peptide flanked by two random amino acids on each side.

Computer modeling indicated that it might be beneficial to stability and folding of the heavy chain to retain four amino acids of the original TT HC CDR2. Specifically, the first three amino acids of the TT heavy chain CDR2 (GLY-ILE-PHE) and the last amino acid of the heavy chain CDR2 (GLY). Thus, the final product would contain these original four amino acids with the TPO mimetic peptide and random flanking amino acids positioned between the first three original amino acids and last original amino acid: GIFXXIEGPTLRQWLAARAXXG (SEQ. ID NO. 119).

The construction of the heavy chain partial CDR2 replacement library was also carried out as described for the previous 2 heavy chain CDR2 libraries with the exception that the specific primer HCR2+ANTI

(5′AGCCAGCCACTGGCGCAGGGTTGGGCCTTCGATMNNMNNGAAGATCCCTCCC ATCCACTC-3′)(SEQ. ID NO. 120) which annealed at the end of the heavy chain FR2 and the primer HCR2+CODE

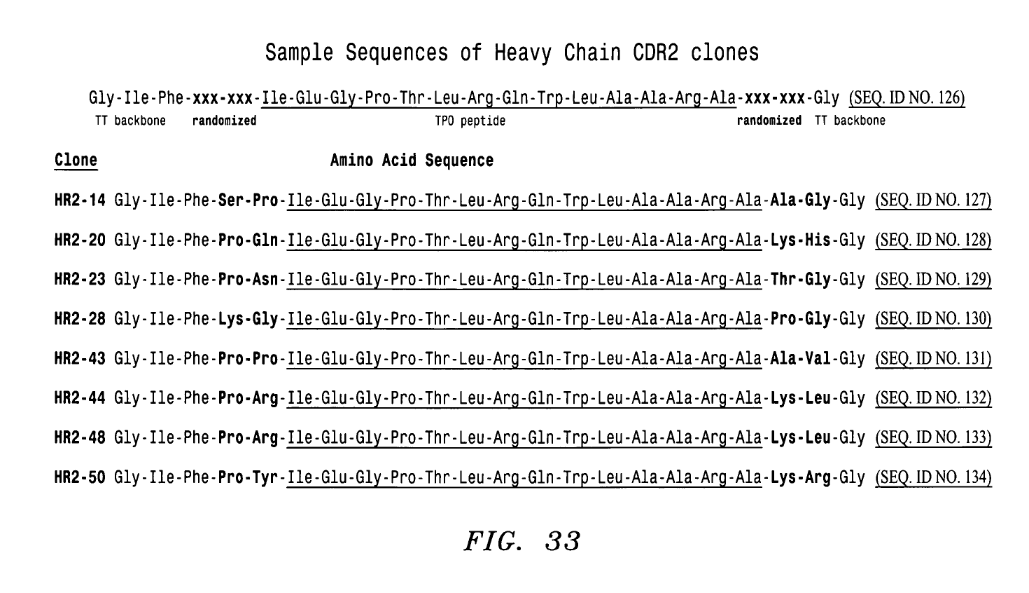

(5′CCAACCCTGCGCCAGTGGCTGGCTGCTCGCGCTNNKNNKGGCAGAGTCACCA TTACCGCG-3′)(SEQ. ID NO. 121) which annealed to the beginning of the heavy chain FR3 were used in place of the previously described HR2 cMPL primers. The heavy chain partial CDR2 replacement library was panned four rounds on 293 EBNA cells transfected with the cMPL-R as described previously. A number of clones with strong binding characteristics were obtained and sequenced (FIG. 33).

The clones of the rounds three and four pool from panning are used as the starting point for insertion of a second TPO mimetic peptide. For example, this library has been combined with the clone X4b containing a TPO mimetic peptide in the heavy chain CDR3 by sub-cloning, to create clones, which contain two TPO mimetic peptides (one in heavy chain CDR2 and one in heavy chain CDR3). These new clones are screened for TPO mimetic activity. In addition, the second peptide may be placed in light chain CDR1, CDR2 or CDR3, or heavy chain CDR1.

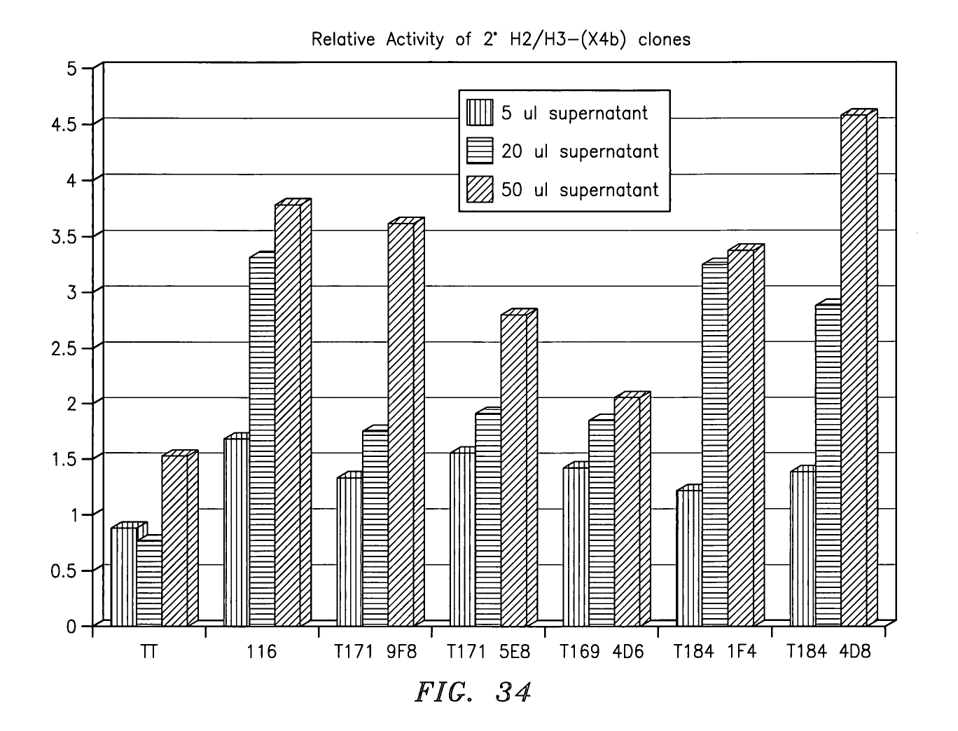

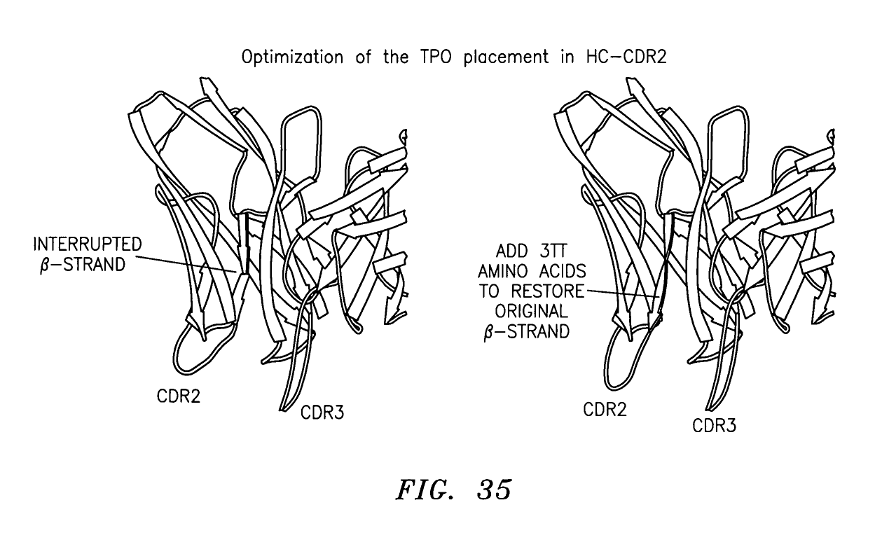

The 2 pools of clones from rounds 3 and 4 were digested with SacII and Spe1 to remove the Tetanus Toxoid HC CDR3. This was replaced by ligating in the X4b HC CDR3 containing the TPO peptide, (see FIGS. 5 and 9), as SacII to Spe1 fragment. These new libraries containing TPO mimetic peptides in both the HC CDR2 and CDR3 were then screened using high through put 96 well format luciferase assays as described below in Example 11. Clones with high levels of activity were then tested for activity in 6 cm luciferase assays (FIG. 34) and protein expression levels were measured by western blotting, as described previously above.

The goal of this example is to optimize the placement of the TPO peptide in the HC-CDR2. In the previous examples, the placement of the TPO peptide interrupts a β-strand (See FIG. 35). The addition of 3 original TT amino acids restores the β-strand. The result is an increased expression of the Fab when only one TPO peptide is grafted in the CDR2 position.

### Example 10

### Conversion of 116 into PRL5 Vector

pING-pAXB 116 DNA was used as a template to generate light and heavy chain fragments of 116 by PCR. The PCR reactions introduced restriction site modifications to the 116 sequence thereby allowing cloning of the fragments into our phage display vector pRL5. In addition, PCR recovery of the heavy chain constant region fragment was done in such a way as to remove a portion of the hinge region sequence. The HC fragment of 116 was recovered as two pieces in order to restore an Apa I site near the juncture of the VH and CH1 domains.

116 Light Chain PCR Rescue & Cloning: PCR was performed using pING-pAXB 116 as the template DNA with primers “116 VL-Fr1 for” (5′ gac gcg cac aac acg GAG CTC GAA ATT GTG CTG ACC CAG AGC 3′)(SEQ. ID NO.135) and “116 CK rev” (5′ aga cag tga gcg ccg TCT AGA a TTA GCA TTC GCC GCG GTT AAA G 3′)(SEQ. ID NO. 136) in a reaction using Advantage 2 High Fidelity PCR kit (BD Biosciences Clontech) with 95° 1 minute followed by 30 cycles of 95° 5 seconds and 68° 1 minute, and finally 68° for 3 minutes. The PCR product was separated from unused primers and reaction components using QlAquick PCR Purification Kit (QIAGEN). The PCR fragment was then digested with restriction enzymes Sac I and Xba I at 37°. QIAquick PCR Purification Kit (QIAGEN) was used to isolate the digested fragment prior to ligation into into pRL5 (Xba I/Sac I digested and gel purified) using T4 DNA Ligase (Invitrogen). The ligation reaction was electroporated into Top10F′ cells (Invitrogen) and grown overnight in SB media at 37° with 50 ug/ml carbenicillin. DNA of the intermediate vector pool pRL5-116 LC was prepared from the resulting bacterial pellet using the QIAGEN Plasmid Maxiprep Kit.

116 Heavy Chain Constant Domain PCR Rescue & Cloning: PCR was performed using pING-pAXB 116 as the template DNA with primers “116 VH-CH1 for” (5′ gac gcg cac aac acg GGC CCG AGC GTG TTT CCG CTG 3′)(SEQ. ID NO.137) and “116 VH-hinge rev” (5′ aga cag tga gcg ccg ACT AGT TTT ATC GCA GCT TTT CGG TT 3′) (SEQ. ID NO.138) in a reaction using Advantage 2 High Fidelity PCR kit (BD Biosciences Clontech) with 95° 1 minute followed by 30 cycles of 95° 5 seconds and 68° 1 minute, and finally 68° for 3 minutes. The PCR product was separated from unused primers and reaction components using QlAquick PCR Purification Kit (QIAGEN). The PCR fragment was then digested with restriction enzymes Apa I at 25° and Spe I at 37° in sequential reactions. QlAquick PCR Purification Kit (QIAGEN) was used to isolate the digested fragment prior to ligation into pRL5-116LC (Apa I/Spe I digested and gel purified) using T4 DNA Ligase (Invitrogen). The ligation reaction was electroporated into TOP10F′ cells, and plated on LB-carbenicillin plates. Bacterial colonies were picked the following day and grown overnight in SB media with 50 ug/ml carbenicillin at 37°. DNA was prepared using QIAprep Spin Miniprep Kit (QIAGEN). All clones were submitted for DNA sequence verification of the entire 116 light chain as well as the constant region fragment of 116 HC. Clone 4E was selected as having the proper sequences (intermediate vector pRL5-116/4E).

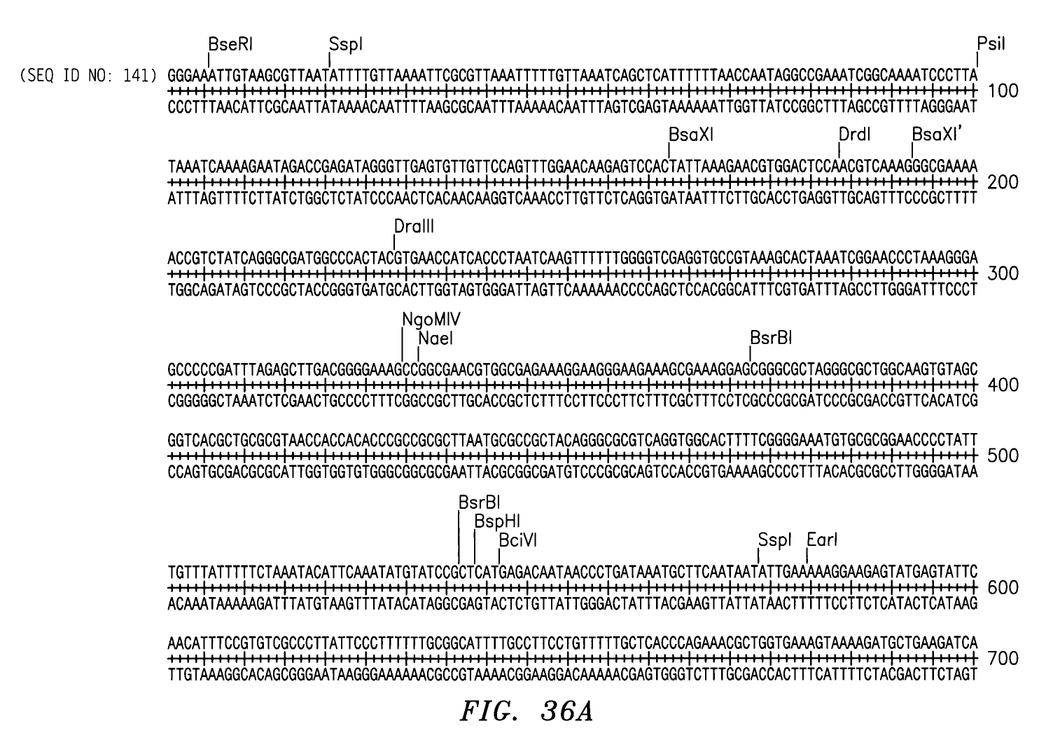

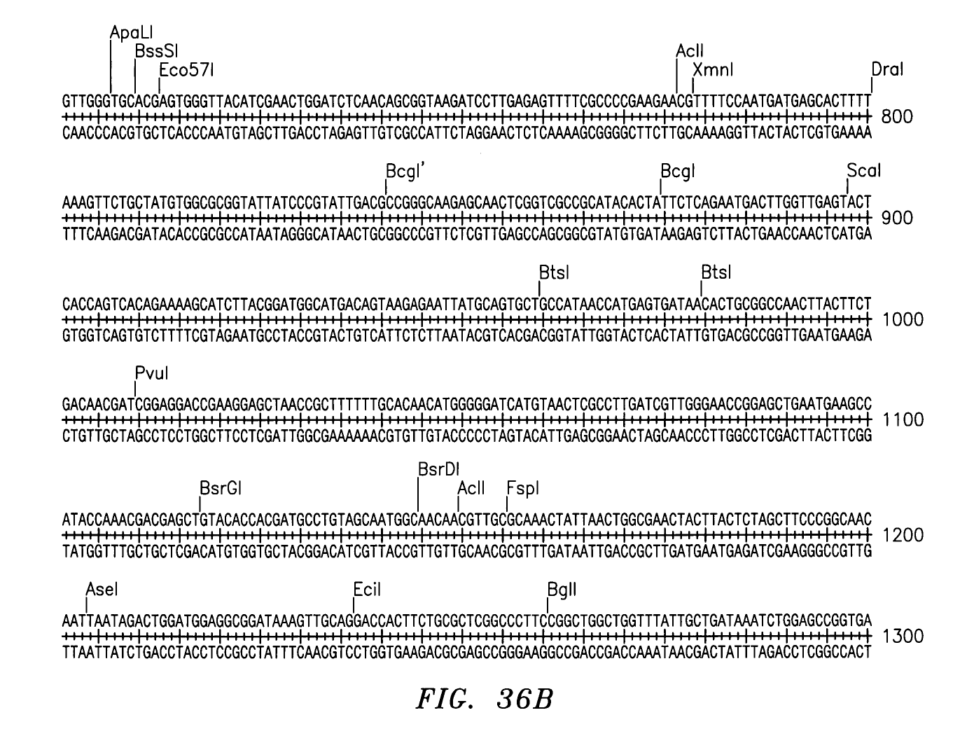

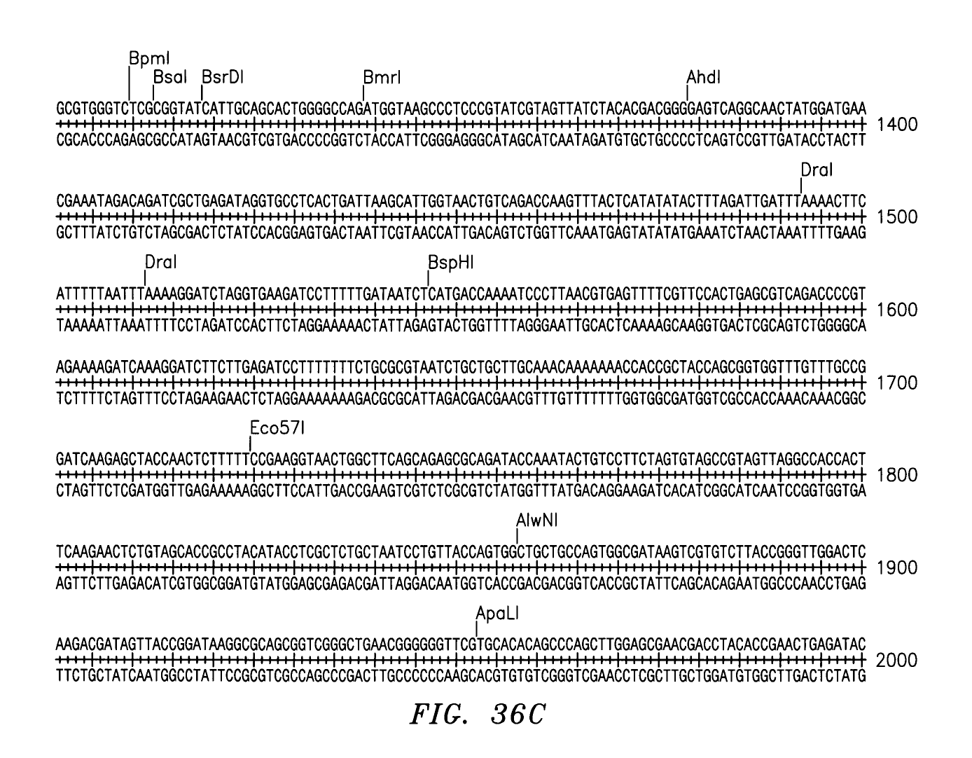

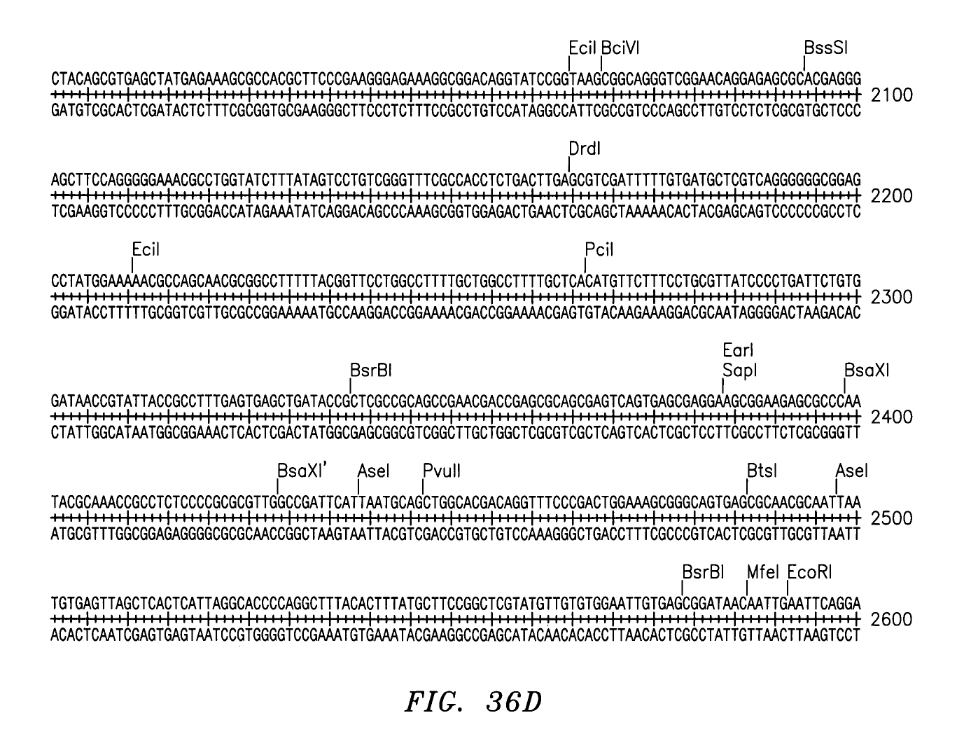

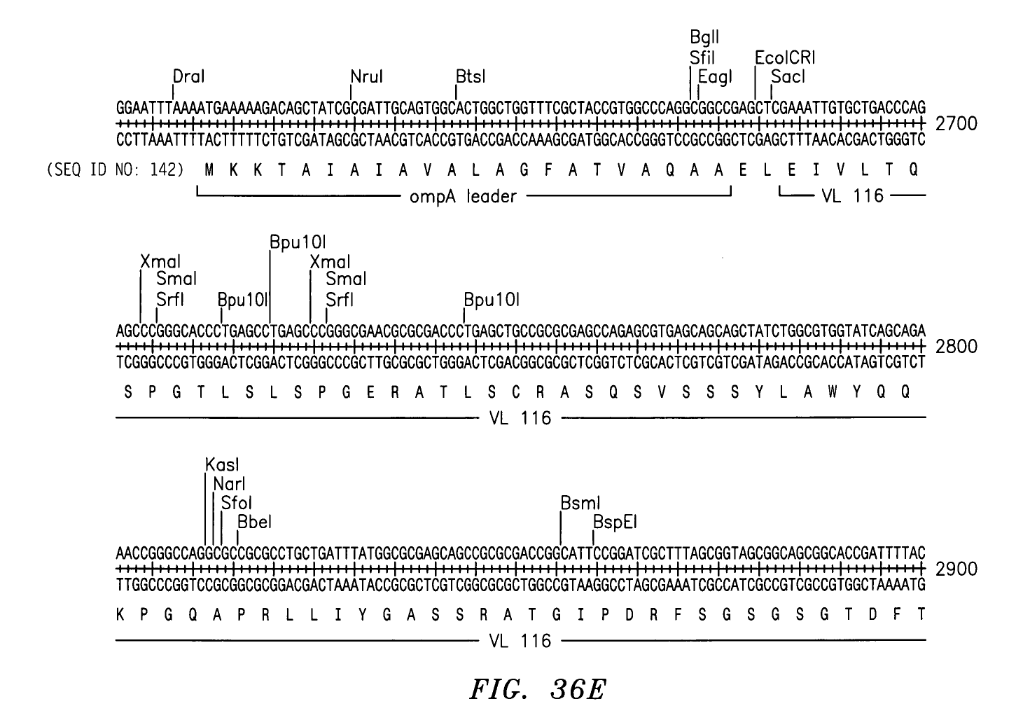

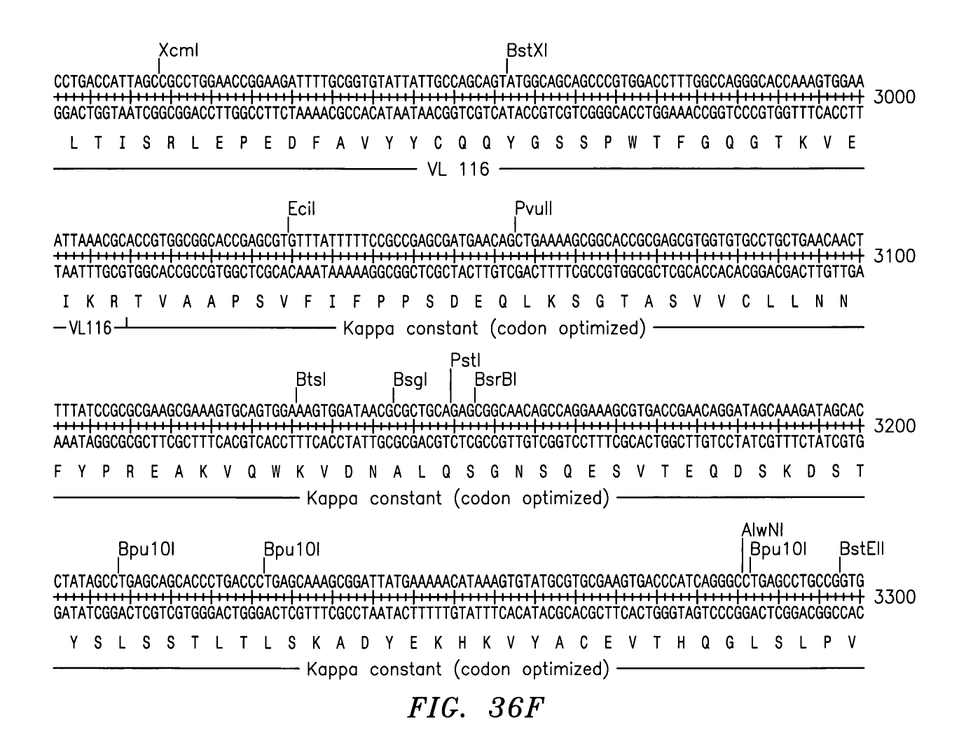

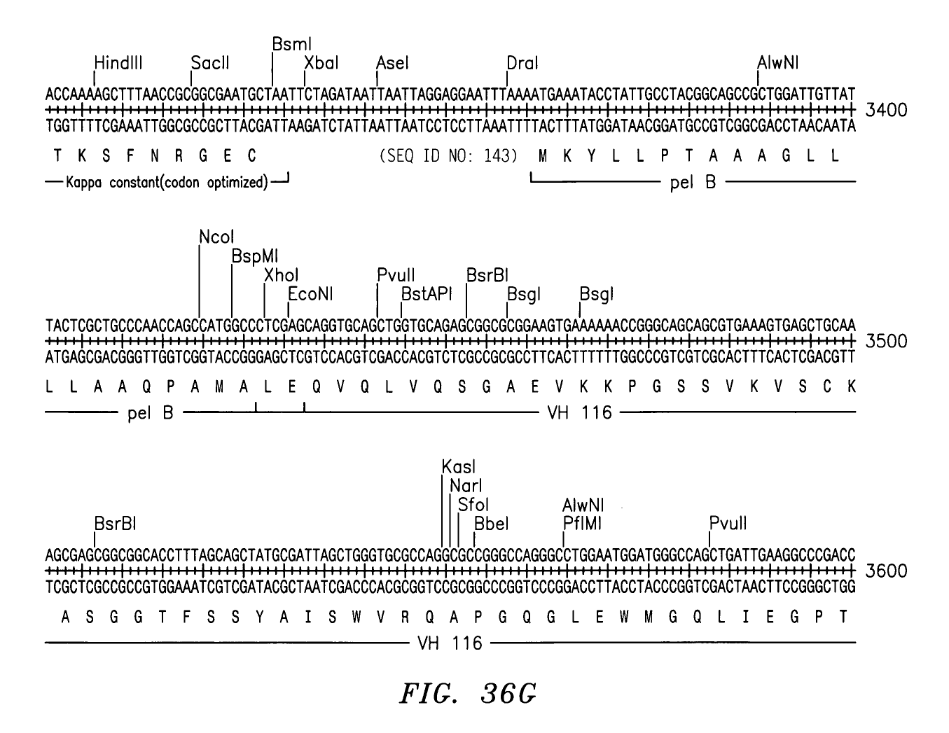

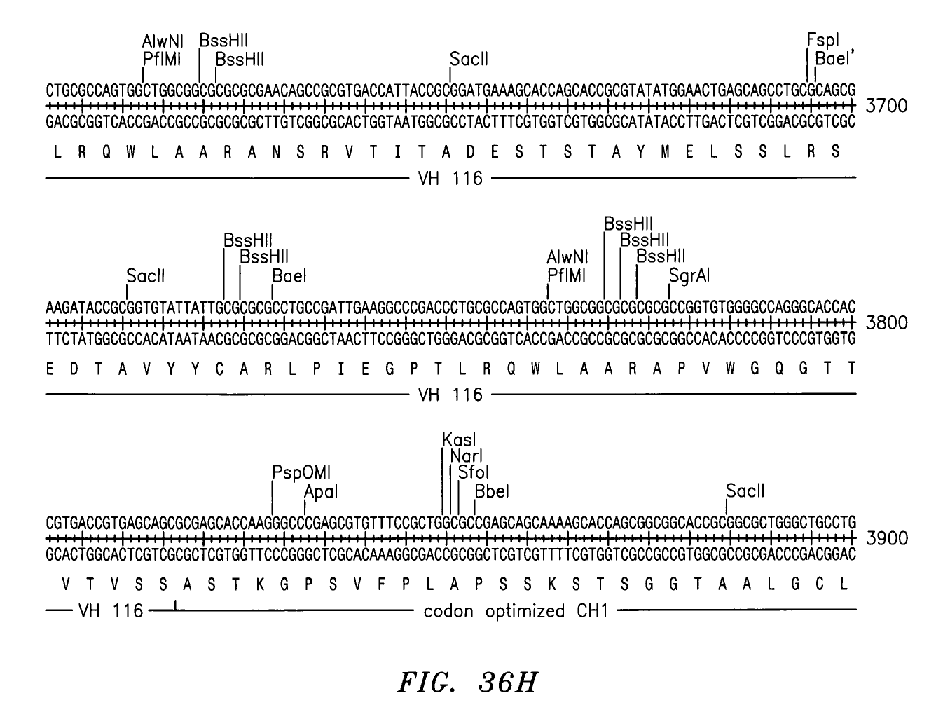

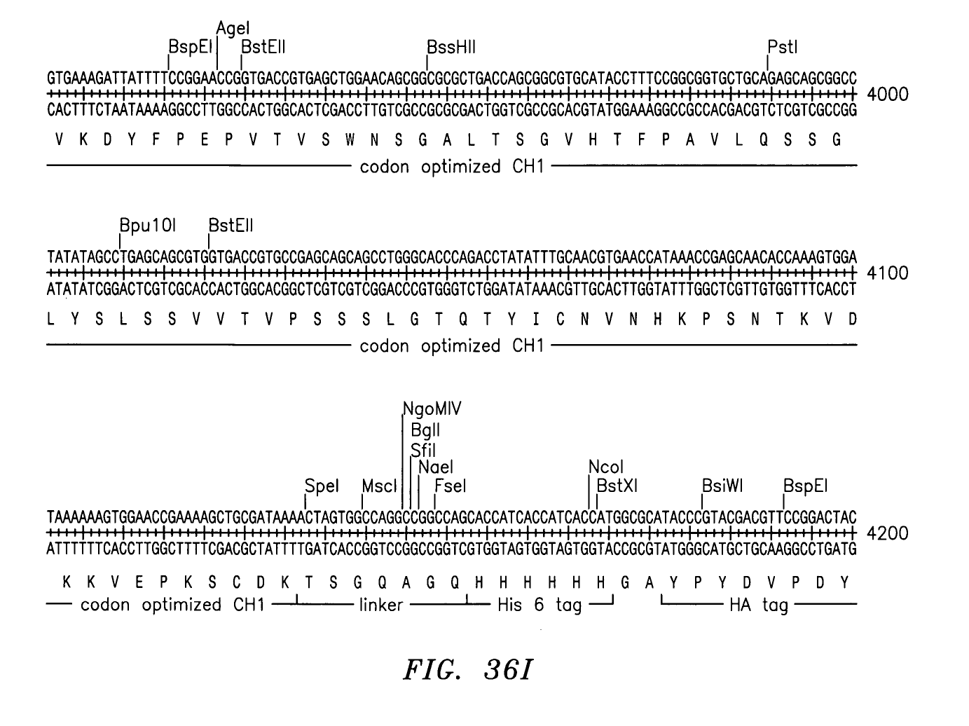

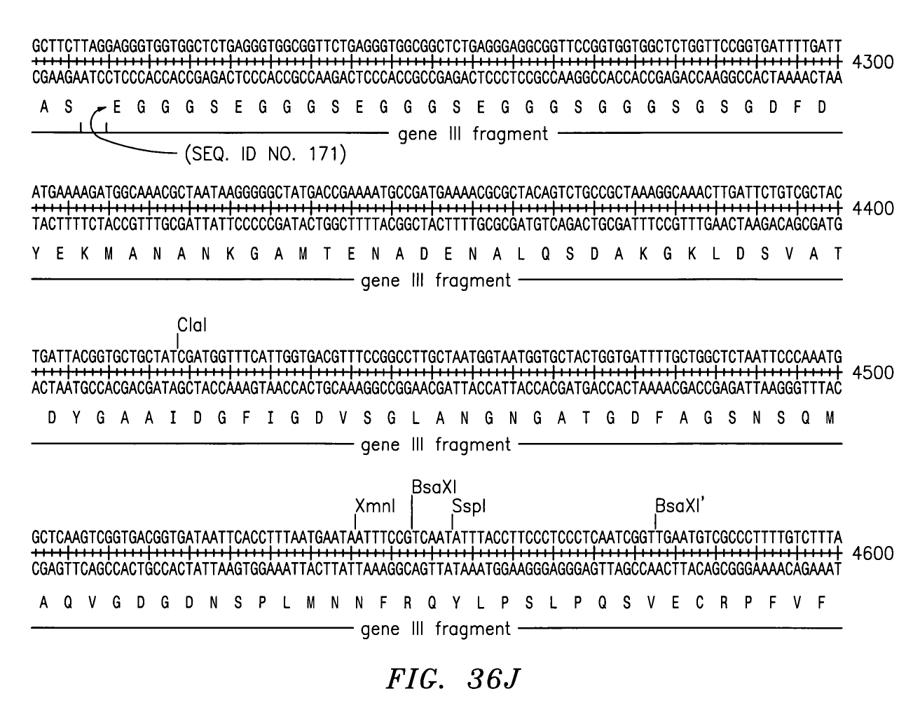

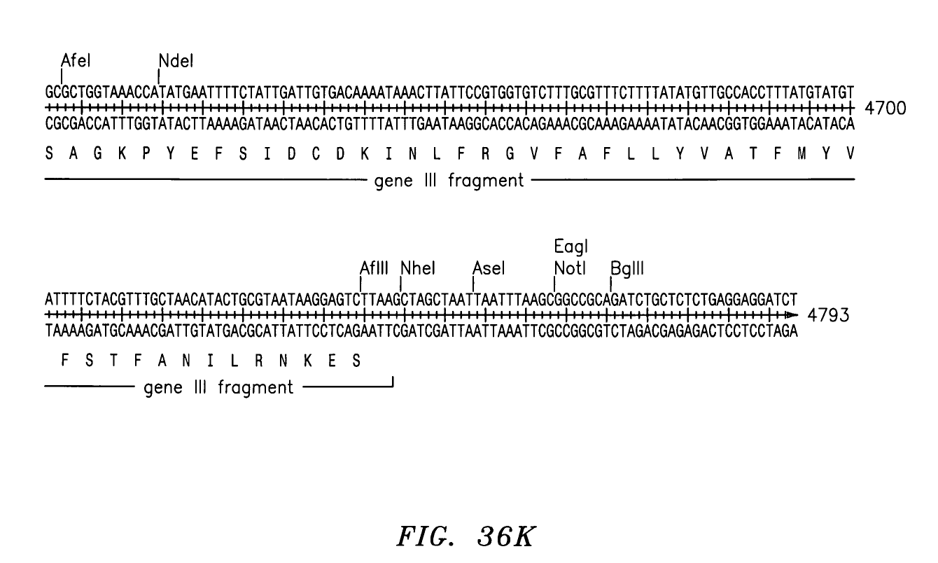

116 Heavy Chain VH PCR Rescue & Cloning: PCR was performed using pING-pAXB 116 as the template DNA with primers “116 VH-Fr1 for” (5′ gag ccg cac gag ccc CTC GAG CAG GTG CAG CTG GTG CAG AG 3′)(SEQ. ID NO.139) and “116 VH-CH1 rev” (5′ gca aag tgt gag GG GCC CTT GGT GCT CGC GCT GCT 3′)(SEQ. ID NO. 140) in a reaction using Amplitaq Gold (Applied Biosystems) and 10.0 ng template vector per 50 μl PCR reaction to promote the accurate replication of the desired PCR fragment. The PCR product was separated from unused primers and reaction components using QIAquick PCR Purification Kit (QIAGEN). The PCR fragment was then digested with restriction enzymes Apa I at 25° and Xho I at 37° in sequential reactions. From there, the DNA fragment was run on an agarose gel and electro-eluted into dialysis tubing prior to ligation into pRL5-116/4E (Xho I/Apa I digested and gel purified) using T4 DNA Ligase (Invitrogen). The ligation reaction was electroporated into TOP10F′ cells (Invitrogen) and plated on LB-Carbenicillin. Bacterial colonies were picked the following day and grown overnight in SB media with 50 ug/ml carbenicillin at 37°. DNA was prepared using QIAprep Spin Miniprep Kit (QIAGEN). All clones were submitted for DNA sequence verification of 116 light chain and heavy chain fragments. Clone pRL5-116F was selected as having the proper DNA sequences. See FIG. 36A-E.

### Example 11

### Modifications to TPO Antibodies

In order to generate the molecular model for the TPO antibody, the heavy and light chain sequences were treated as two separate entities. Each chain was then used as a query sequence to search the protein data bank for antibodies with similar primary structure using the program psi-blast. The structures of the top scoring sequences for both the light and heavy chain were combined to create a composite antibody Fab structure. The back bone of this composite structure was then used as a template on which the residues unique to TPO could be added and any amino acid insertions or deletions could be made using SwissModel. Once the necessary changes were made to convert the composite Fab structure to a model of TPO the entire TPO model was minimized against an energy score using conjugate gradient minimization techniques in SwissModel. Upon achieving a local minimum the model was deemed complete and was used for analysis purposes. Proposed mutations to TPO were first modeled onto the backbone of the model and possible interactions were mapped. Side-chain rotomers of the residue being mutated as well as any residues within interacting distance of the residue in question were evaluated. Analysis of the TPO antibody in this fashion allowed us to make specific suggestions about the effects of mutations already carried out in vitro as well as to predict beneficial mutations.

Computer modeling was used to examine the 116 Fab antibody structure to help direct possible mutational changes within the molecule in order to promote stability and improve the specific activity and/or the Fab protein yield produced by the bacteria. Using computer protein modeling it was determined that in the three dimensional structure of the 116 molecule, two of the germline changes occurring within the heavy chain are positioned in between the CDR loop structures (CDR2 and CDR3), which each contain a TPO mimetic peptide. These two germline changes occur where two adjacent asparagines at the end of framework 1 and the start of CDR1 respectively in the original Tetanus Toxoid backbone ( Kabat amino acids position numbers 30 and 31) were both changed to serine residues in the 116 molecule. Computer modeling indicated that the conversion of these asparagines to serine residues created unfavorable contacts with the CDR loops containing TPO peptides, and that conversion back to asparagines or some other amino acid might be favorable. Thus in the first experiment these two serines were converted back to asparagines within 116 in the pRL5 vector. In an additional separate experiment the two serine residues were randomized to create a library within the pRL5 116 backbone.

Overlap PCR, as decribed previously in examples 1 and 2, was used to create the new randomized library as well as the single mutant with conversion of the two asparagines back to serines. For the randomized library, the fragment A was created using the vector specific forward primer Lead VH (SEQ. ID NO.13) as previously described (Example 2), and the reverse primer 116REV (5′ AAA GGT GCC GCC GCT CGC TTT GCA 3′)(SEQ. ID NO.144) that annealed to framework 1 just upstream of the two serines. Fragment B was created using the forward primer 116SSTOXX (5′ GGC GGC ACC TTT NNK NNK TAT GCG ATT AGC TGG GTG CGC CAG 3′)(SEQ. ID NO. 145), which anneals to the end of framework 1 and CDR1, and the vector specific NDP reverse primer (SEQ. ID. NO. 15) as previously described (Example 2). The 116SSTOXX forward primer contained the two randomized positions (NNK) and contained a 12 bp region complementary to the 116REV primer for overlap pcr extension. After the individual fragments were generated they were gel purified and were combined for overlap pcr extension using the Lead VH and NDP primers to generate a library of full heavy chain fragments of 116, as previously described (Example 2). These heavy chains were then digested with Xho 1 and Spe 1 as previously described (Example 2) and cloned back into the 116 pRL5 vector which was similarly digested. The individual mutant converting the two germline serines back into their original asparagines was similarly constructed with the exception that the forward primer 116SSTONN 116SSTOXX (5′ GGC GGC ACC TTT ACC ACC TAT GCG ATT AGC TGG GTG CGC CAG 3′)(SEQ. ID NO.146), was used instead of the 116SSTOXX primer.

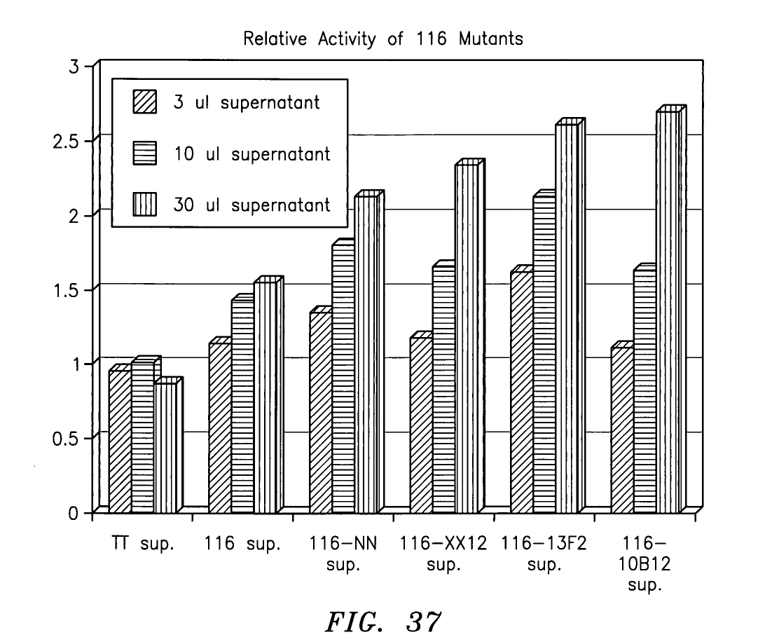

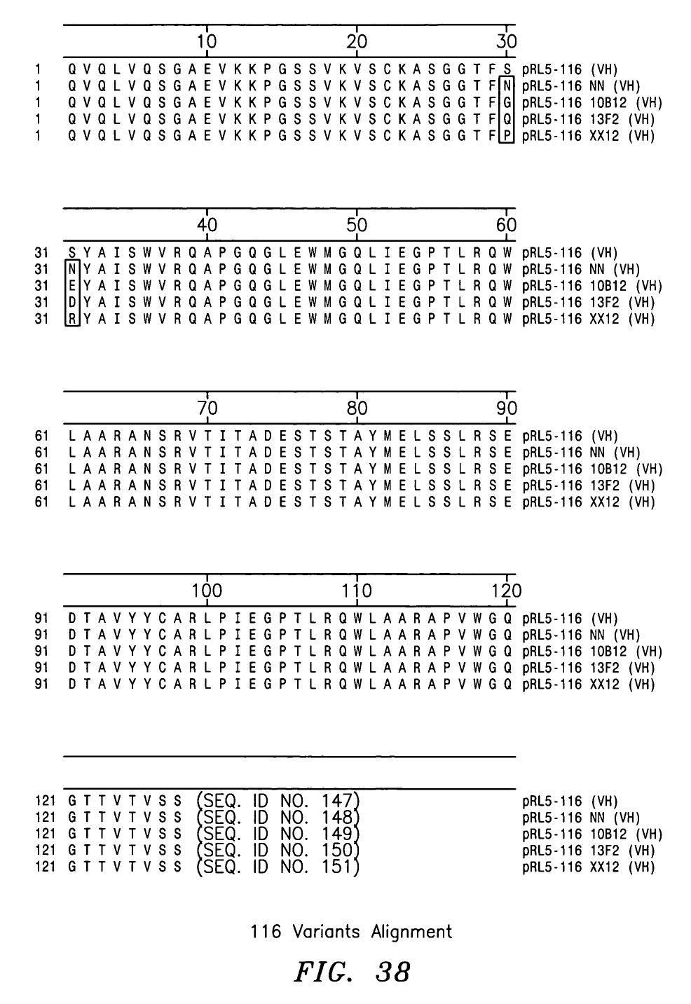

Bacterial supernatants from this library were then screened using high throughput luciferase activity assays in 96 well format and raw bacterial supernatants. For these assays NIH 3T3 cells were transiently transfected with the cMpl-R and a Fos promoter/luciferase reporter plasmid as described in Example 1. The following day cells were split into 96 well dishes at 10,000 cells per well. After 4-6 hrs, during which time cells attached and adhered to the plate, cells were then washed once with PBS and the media was changed to low serum (5%) media for and additional 24 hrs. Bacterial overnights of the test clones grown in 96 well plates were spun down at 2000 rpm. Cleared supernatants were diluted 1 to 10, and 5 ul of the diluted supernatant was added the wells containing the transiently transfected 3T3 cells. Cells were treated for 6 hrs at 37° C. to allow for Luciferase induction. Cells were harvested and luciferase activity was measured using the Bight-Glo Luciferase Assay System from Promega, following the kit instructions and measuring activity using the Genesis Freedom 200 robot from Tecan and the Tecan Spectrfluor Plus reader. Approximately 1200 clones were screened and compared in activity to the original 116. Supernatants from clones, which gave equivalent or higher activities, were then examined by western blot to determine the amount Fab protein produced. Clones which appeared to produce equivalent or higher levels of Fab protein in the bacterial supernatants, as compared to 116 supernatants; and had equivalent or higher luciferase activities in the 96 well assays, were further tested in 6 cm luciferase assays (FIG. 37), as previously described in Example 1. Test clone bacterial supernatants were again compared with 116 bacterial supernatants in these assays. Clones indicated by these assays were then sequenced to determine which amino acids replaced the two serines and were selected for from the randomized library (FIG. 38). In addition to the 116 clone made separately with two asparagines, two clones from the randomized library were selected, which also had asparagines in both of these two positions, although different codons were used in these clones coding for asparagine. In addition, clones containing proline and arginine, glycine and glutamate, and glutamine and aspartate were selected. The clone names and amino acids respectively are 116-NN (asparagine-asparagine), 116-XX12 (proline-arginine), 116-10B12 (glycine-glutamate), and 116-13F2 (glutamine-aspartate) (See FIG. 38). These clones can be cloned into an expression vector (such as, for example the XOMA pING vector system) for purification of the Fab protein. The purified antibodies can be compared to 116 purified in the same manner to accurately compare the specific activities of parental 116 to the selected mutant forms.

### Example 12

### GLP-1 Peptide-engrafted Antibody Library

Library Construction.

Primers were designed to insert GLP-1 peptide into the heavy chain CDR3 of a human tetanus toxoid (TT) specific antibody in pRL4 vector (pRL4-TT) by overlap PCR. Two amino acids franking the peptide sequence on both 5′ and 3′ ends were randomized to gain optimum presentation of each peptide at HCD3. Initial PCR amplification consisted of 2 separate PCR amplifications. Primers used for these amplifications are listed in Table A below. One reaction used 5′ Lead VH primer that anneals to the Pel B leader sequence in the pRL4 vector paired with 3′ primers GLP-1TTH3-R and GLP-1 int-R. Second reaction used 3′ SeqG3-R primer that anneals to the gene III sequence in the pRL4 vector paired with 5′ primers GLP-1 int-F and GLP-1TTH3-F. 3′ primer GLP-1TTH3-R was designed to contain part of the peptide sequence, 6 randomized nucleotides, and a portion that anneals to the framework 3 region of heavy chain pRL4-TT. Another 3′ primer GLP-1 int-R was designed to contain part of the peptide sequence, which is longer than the part in the GLP-1TTH3-R primer to allow the extension of the amplified product. They were used at 0.04 μM and 0.4 μM in the reaction, respectively. 5′ primers GLP-1TTH3-F was designed to contain part of the peptide sequence, 6 randomized nucleotides, and a portion that anneals to the framework 4 region of heavy chain pRL4-TT. Another 5′ GLP-1 int-F primer is reverse complement to the 3′ GLP-1 int-R primer, which allows the overlap PCR. For each reaction, approximately 10 ng of pRL4-TT antibody DNA was mixed with primers, dNTPs, Advantage 2 HF polymerase mix and its 10× buffer (BD Biosciences Clontech). The sample was heat denatured at 95° C. for 1 minute then cycled 30 times through 95° C. for 5 seconds and 68° C. for 1 minute. This is followed by an additional 3 minutes at 68° C. and a 4° C. hold. PCR products were gel purified and the overlap PCR was performed. The purified DNA was mixed with 5′ H2H3SSTOXX-F primer and 3′ SeqG3-R primer, dNTPs, Advantage 2 HF polymerase mix and its 10× buffer (BD Biosciences Clontech). The sample was heat denatured at 95° C. for 1 minute then cycled 30 times through 95° C. for 5 seconds and 68° C. for 1 minute. 5′ H2H3SSTOXX-F primer anneals to the framework 1 region of heavy chain pRL4-TT. In a separate reaction, pRL4-TT was amplified with 5′ LeadVH primer and 3′ H2H3SSTOXX-R primer. 3′ H2H3SSTOXX-R primer has a portion that anneals to the framework 1 region of heavy chain pRL4-TT, 6 randomized nucleotides, and a reverse complement sequence to the 5′ H2H3SSTOXX-F which allows the second overlapping PCR for each peptide insertion into pRL4-TT heavy chain CDR3. Each amplified product is gel purified and used as templates for the second overlapping PCR. The purified DNA was mixed with 5′ LeadVH primer and 3′ SeqG3-R primer, dNTPs, Advantage 2 HF polymerase mix and its 10× buffer (BD Biosciences Clontech). The sample was heat denatured at 95° C. for 1 minute then cycled 30 times through 95° C. for 5 seconds and 68° C. for 1 minute.

The amplified product was inserted into pRL4-TT antibody at Xho I/Spe I sites The peptide-engrafted libraries were designated GLP-1 XXTTH3. The resultant library size was 1.0×10⁹, high enough to cover the calculated library size by amino acids randomized at 6 positions (6.4×10⁷).

TABLE A

| | |
|---|---|
| LeadVH: | |
| 5′ GCT GCC CAA CCA GCC ATG GCC 3′ | (SEQ ID NO. 179) |
| SeqG3-R: | |
| 5′ATC AAA ATC ACC GGA ACC AGA GC | (SEQ ID NO. 180) |
| 3′ | |
| GLP-1TTH3-R: | |
| 5′TTC CAA ATA AGA ACT TAC ATC ACT | (SEQ ID NO. 181) |
| GGT AAA GGT CCC TTC AGC ATG MNN | |
| MNN TCT CGC ACA ATA ATA TAT GGC 3′ | |
| GLP-1TTH3-F: | |
| 5′ GGC CAA GCT GCC AAG GAA TTC ATT | (SEQ ID NO. 182) |
| GCT TGG CTG GTG AAA GGC CGA NNK | |
| NNK TGG GGC CAA GGG ACC ACG GTC 3′ | |
| GLP-1 int-F: | |
| 5′ CAG TGA TGT AAG TTC TTA TTT GGA | (SEQ ID NO. 183) |
| AGG CCA AGC TGC CAA GGA ATT CAT TG | |
| 3′ | |
| GLP-1 int-R: | |
| 5′ CAA TGA ATT CCT TGG CAG CTT GGC | (SEQ ID NO. 184) |
| CTT CCA AAT AAG AAC TTA CAT CAC TG | |
| 3′ | |
| H2H3SSTOXX-F: | |
| 5′ TAT GCC ATC AGC TGG GTG CGA CAG | (SEQ ID NO. 185) |
| 3′ | |
| H2H3SSTOXX-R | |
| 5′ TCG CAC CCA GCT GAT GGC ATA MNN | (SEQ ID NO. 186) |
| MNN GAA GGT GCC TCC AGA AGC CCT 3′ | |

Fab expression of each clone was checked by ELISA and insertion of peptide was checked by 5′ peptide-specific primer: GLP-1 int-F paired with 3′ SeqG3-R primer.

Selection of the GLP-1 Peptide Heavy Chain CDR3 Library

In order to select for the optimal peptide display, panning is performed on GLP-1 receptor either recombinantly expressed and immobilized on microtiter wells or RINm5F insulinoma cells. Four rounds of panning on the GLP-1 receptor are performed using essentially the same technique described in Example 1, above.

Examination Of Insulinotropic Activity Candidates By Perfusion Experiments

Functional screening of insulinotropic activity of isolated clones is conducted in three separate ways. Rat pancreas perfusion experiments and/or stimulation of cyclic AMP production evaluated using cultured RINm5F insulinoma cells is performed using the methods described in Watanabe, et al., J Endocrinol. Jan;140(1):45-52 (1994); Gallwitz ey al., J Mol Endocrinol. Jun;10(3):259-68 (1993); Richter, et al., J Endocrinol., Sep;126(3):445-50 (1990); Gallwitz et al., J Mol Endocrinol., Aug;5(1):33-9 (1990); Flatt et al., Diabetes Res., Feb;13(2):55-9 (1990); and/or Goke, J Endocrinol., Mar;116(3):357-62 (1988). Screening is also done by evaluating binding affinity to GLP-1 receptor using techniques known to those skilled in the art.

Several Fabs, as bacterial supernatants, are tested for reactivity to the original antigen tetanus toxoid in order to determine if that binding specificity is retained.

Sequence analysis of Fab clones which specifically bound to the GLP-1 receptor is performed. Functional Fabs are converted to full IgG using techniques within the purview of those skilled in the art and re-tested by functional screening, as described above. IgG are then tested in an animal model using ApoE null mice and/or ob/ob mice.

### Example 13

### ANP and BNP Peptide-engrafted Antibody Libraries

Library Construction.

Primers were designed to insert each peptide into the heavy chain CDR3 of a human tetanus toxoid (TT) specific antibody in pRL4 vector (pRL4-TT) by overlap PCR. Two amino acids franking the peptide sequence on both 5′ and 3′ ends were randomized to gain optimum presentation of each peptide at HCD3. Initial PCR amplification consisted of 2 separate PCR amplifications. Primers used for these amplifications are listed in Table B below. One reaction used 5′ Lead VH primer that anneals to the Pel B leader sequence in the pRL4 vector paired with 3′ primers ANPTTH3-R (ANP) and BNPTTH3-R (BNP). Second reaction used 3′ SeqG3-R primer that anneals to the gene III sequence in the pRL4 vector paired with 5′ primers ANPTTH3-F (ANP) and BNPTTH3-F (BNP). 3′ primers ANPTTH3-R and BNPTTH3-R were designed to contain part of the peptide sequence, 6 randomized nucleotides, and a portion that anneals to the framework 3 region of heavy chain pRL4-TT. 5′ primers ANPTTH3-F and BNP-TTH3-F were designed to contain part of the peptide sequence, 6 randomized nucleotides, and a portion that anneals to the framework 4 region of heavy chain pRL4-TT. ANPTTH3-R and ANPTTH3-F primers, and BNPTTH3-R and BNPTTH3-F primers had a complementary 25-base overlapping portion that can be utilized for the overlapping PCR, respectively. For each reaction, approximately 10 ng of pRL4-TT antibody DNA was mixed with primers, dNTPs, Advantage 2 HF polymerase mix and its 10× buffer (BD Biosciences Clontech). The sample was heat denatured at 95° C. for 1 minute then cycled 30 times through 95° C. for 5 seconds and 68° C. for 1 minute. This is followed by an additional 3 minutes at 68° C. and a 4° C. hold. PCR products were gel purified and the overlap PCR was performed. The purified DNA was mixed with 5′ H2H3SSTOXX-F primer and 3′ SeqG3-R primer, dNTPs, Advantage 2 HF polymerase mix and its 10× buffer (BD Biosciences Clontech). The sample was heat denatured at 95° C. for 1 minute then cycled 30 times through 95° C. for 5 seconds and 68° C. for 1 minute. 5′ H2H3SSTOXX-F primer anneals to the framework 1 region of heavy chain pRL4-TT. In a separate reaction, pRL4-TT was amplified with 5′ LeadVH primer and 3′ H2H3SSTOXX-R primer. 3′ H2H3SSTOXX-R primer has a portion that anneals to the framework 1 region of heavy chain pRL4-TT, 6 randomized nucleotides, and a reverse complement sequence to the 5′ H2H3SSTOXX-F which allows the second overlapping PCR for each peptide insertion into pRL4-TT heavy chain CDR3. Each amplified product is gel purified and used as templates for the second overlapping PCR. The purified DNA was mixed with 5′ LeadVH primer and 3′ SeqG3-R primer, dNTPs, Advantage 2 HF polymerase mix and its 10× buffer (BD Biosciences Clontech). The sample was heat denatured at 95° C. for 1 minute then cycled 30 times through 95° C. for 5 seconds and 68° C. for 1 minute.

The amplified product was inserted into pRL4-TT antibody at Xho I/Spe I sites The peptide-engrafted libraries were designated ANP XXTTH3 and BNP XXTTH3. The resultant library size was 9.4×10⁸ (ANP XXTTH3), and 5.4×10⁸ (BNP XXTTH3) high enough to cover the calculated library size by amino acids randomized at 6 positions (6.4×10⁷).

TABLE B

| | |
|---|---|
| LeadVH: | |
| 5′ GCT GCC CAA CCA GCC ATG GCC 3′ | (SEQ ID NO. 179) |
| SeqG3-R: | |
| 5′ATC AAA ATC ACC GGA ACC AGA GC | (SEQ ID NO. 187) |
| 3′ | |
| ANP TTH3-R: | |
| 5′ CTC TGG GCT CCA ATC CTG TCC ATC | (SEQ ID NO. 188) |
| CTG CCC CCG AAG CAM NNM NNT CTC | |
| GCA CAA TAA TAT ATG GC 3′ | |
| ANP TTH3-F: | |
| 5′ GGA TGG ACA GGA TTG GAG CCC AGA | (SEQ ID NO. 189) |
| GCG GAC TGG GCT GTN NKN NKT GGG | |
| GCC AAG GGA CCA CGG TC 3′ | |
| BNP TTH3-R: | |
| 5′ CTG GAG GAG CTG ATC CGG TCC ATC | (SEQ ID NO. 190) |
| TTC CTC CCA AAG CAM NNM NNT CTC | |
| GCA CAA TAA TAT ATG GC 3′ | |
| BNP TTH3-F: | |
| 5′ AGA TGG ACC GGA TCA GCT CCT CCA | (SEQ ID NO. 191) |
| GTG GCC TGG GCT GCN NKN NKT GGG | |
| GCC AAG GGA CCA CGG TC 3′ | |
| H2H3SSTOXX-F: | |
| 5′ TAT GCC ATC AGC TGG GTG CGA CAG | (SEQ ID NO. 192) |
| 3′ | |
| H2H3SSTOXX-R: | |
| 5′ TCG CAC CCA GCT GAT GGC ATA MNN | (SEQ ID NO. 193) |
| MNN GAA GGT GCC TCC AGA AGC CCT 3′ | |

Fab expression of each clone was checked by ELISA and insertion of peptide was checked by 5′ peptide-specific primers: ANP int-F and BNP int-F paired with 3′ SeqG3-R primer. The sequence of ANP int-F: is 5′ GGA TGG ACA GGA TTG GAG CCC AGA G 3′ for ANP XXTTH3 library and BNP int-F is 5′ AGA TGG ACC GGA TCA GCT CCT CCA G 3′ for BNP XXTTH3 library.

Selection of the hBNP Heavy Chain CDR3 Library

In order to select for the optimal peptide display, panning is performed The library is panned on a fusion protein of extracellular domain of NPR-A and IgG Fc (NPR-A ECD-Fc) and negatively selected on recombinant natriuretic peptide receptor type A (NPR-A) or selected on SK-N-SH cells or SK-N-BE cells. For the panning on a fusion protein, the library-phage is suspended in a blocking solution (i.e. 4% nonfat dry milk/PBS). Selection of binders is performed on microtiter wells coated with NPR-A ECD-Fc (10 μg/ml). Screening is done by ELISA on NPR-A ECD Fc. Fifty micro-liter of each protein and a control protein, ovalbumin (Pierce) are coated on microtiter wells at 4 μg/ml in PBS at 4° C. overnight. Wells are blocked with 100μl of 4% nonfat dry milk/PBS at room temperature for 30 min. Milk is discarded and the wells are incubated with Fabs for 1 hr at 37° C. Wells are washed with PBS and the binding of Fab is detected with alkaline-phosphatase conjugated goat anti-human IgG F(ab′)₂ antibody (Pierce)(1:500) in 1% BSA/PBS at 37° C. for 1 hr. Specific binders on NPR-A ECD-Fc are selected and tested in a cell-based assay. For the panning of SK-N-SH cells, the library-phage are suspended in a blocking solution (i.e. 1%BSA/PBS) with protein inhibitor and dialyzed for 2 hours in PBS. Selection of binders is performed on SK-N-SH cells in a tissue culture plate. Screening is performed by flow cytometry. Each Fab is mixed with cells on ice for 1 hr. cells are washed with PBS and the binding of Fab is detected with fluorescent isothiocyanate conjugated goat anti-human IgG F(ab′)₂ antibody (Pierce)(1:100) in 1% BSA/PBS on ice for 20 min. Specific binders to SK-N-SH are selected.

Several Fabs, as bacterial supernatants, are tested for reactivity to the original antigen tetanus toxoid in order to determine if that binding specificity is retained.

Examination Of Natriuretic Peptide Receptor Type A (NPRA) Activity

Functional screening of isolated clones is conducted using a cell based assay system for the evaluation of human brain natriuretic peptide (hBNP) activity on natriuretic peptide receptor type A (NPRA)-bearing cells (neuroblastoma cell line, SK-N-SH). Specifically, 3H-thymidine—SK-N-SH cells are plated at 20,000/ml in EMEM containing 10% FBS, 2 mM L-glutamine, 1 mM sodium pyruvate, 0.1 mM nonessential amino acids, 1.5g/l sodium bicarbonate and antibiotics in 96 well plates adding 200 μl to each well. After 4 h, medium is switched to serum-free conditions. 24 h later, cells are stimulated with peptides (hBNP-32 and CNP from Sigma) or selected Fab clones. 48 h later, 1 mCi 3H-thymidine is added per well. Cells are harvested using a Universal Cell Harvester (Packard) after 20 h and integrated radioactivity is measured using a Topcount (Packard).

Cyclic AMP and GMP measurements are made as follows: cells (80,000/well) are cultured for 3 days in 24-well plates. Medium is replaced with serum-free medium containing 0.1 mM isobutylmethylxanthine and, in the case of cAMP measurement, with or without 10 μM forskolin (Sigma). After incubation for 15 min at 37° C., Fabs are added and cells are incubated further for 15 min at 37° C. Cells are lysed in 6% trichloroacetic acid solution, and radioimmunoassays are performed according to the manufacturer's protocol (PerkinElmer Life Sciences).

Sequence analysis of Fab clones which specifically bind to the NPR-A ECD-Fc fusion protein is performed. Functional Fabs are converted to full IgG using techniques within the purview of those skilled in the art and re-tested by functional screening, as described above. These IgG antibodies are tested in an animal model, such as that described by Clemens et al., Pharmacol Exp Ther. Oct;287(1):67-71 (1998) to show the effects of hBNP antibodies in New Zealand White rabbits in normotensive and acute norepinephrine-induced hypertensive states. Antibodies are administered to anesthetized rabbits intravenously or subcutaneously and the kinetics and the effect on plasma cyclic GMP, diuresis and natriuresis and a decrease in systolic blood pressure are compared with hBNP (1, 3,10, and 30 microg/kg).

Hemodynamic and Renal Measurements

Animals are anesthetized with pentobarbital infusion (40-60 mg/kg) via ear vein. Throughout the experiment, body temperature is maintained on a circulating heating pad at 36° C. Rabbits are administered a continuous infusion of 0.9% NaCl (6 ml/kg/hr) and pentobarbital (10 mg/kg/hr) via the marginal ear vein for the duration of the experiment. A catheter (PE90) is advanced 10 cm through the femoral artery and connected to a Grass Physiograph and a CODAS (DATAQ, Akron, Ohio) data capture system. Cardiovascular data are monitored continuously, and blood pressures (mean, systolic, diastolic) is expressed as the average value during 10-min periods. Urine is collected from the bladder with a 12-Fr Foley catheter in 20-min intervals. The rates of urine flow and urine sodium excretion are quantitated by weight and flame photometry (Instrumentation Laboratory, Lexington, Mass.), respectively. Drug treatment is either vehicle (0.9% NaCI, 1 ml/kg) or hBNP (30 pg/kg, 1 ml/kg) or mimetic antibodies (150 μg/kg, 1 ml/kg). One group of rabbits have hBNP or mimetic antibodies or vehicle delivered via a catheter placed in the left femoral vein (intravenous drug delivery protocol) while the second group of animals have hBNP or mimetic antibodies or vehicle delivered by subcutaneous injection between the shoulder blades (subcutaneous protocol).

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended herein.

What is claimed is:

1. An immunoglobulin molecule or fragment thereof comprising a region where amino acid residues corresponding to at least a portion of a complementarity determining regions (CDR) is replaced with a peptide selected from the group consisting of hBNP, hBNP mimetics, GLP-1, GLP-1 mimetics, GLP-2, GLP-2 mimetics, exendin, exendin mimetics, glucagons, glucagon mimetics and PACAP-38.

2. An immunoglobulin molecule or fragment thereof according to claim 1 further comprising at least one flanking sequence including at least one amino acid covalently linked to at least one end of the peptide.

3. An immunoglobulin molecule or fragment thereof according to claim 1 wherein the immunoglobulin molecule fragment is selected from the group consisting of Fab fragment, F(ab′)₂ fragment and ScFv fragment.

4. An immunoglobulin molecule or fragment thereof according to claim 1 wherein the immunoglobulin molecule is a full IgG molecule.

5. An immunoglobulin molecule or fragment thereof according to claim 1 wherein at least a portion of two CDRs are replaced with a peptide.

6. An immunoglobulin molecule or fragment thereof according to claim 5 wherein the two CDRs are both located on a heavy chain.

7. An immunoglobulin molecule or fragment thereof according to claim 5 wherein the two CDRs are a CDR3 of a heavy chain and a CDR2 of a heavy chain.

8. An immunoglobulin molecule or fragment thereof according to claim 1 wherein the immunoglobulin molecule or fragment thereof is human.

9. An immunoglobulin molecule or fragment thereof according to claim 1 wherein the immunoglobulin molecule or fragment thereof is anti-tetanus toxoid.

10. Nucleic acid encoding an immunoglobulin molecule or fragment thereof according to claim 1.

11. An expression vector comprising nucleic acid according to claim 10.

12. A host cell transformed with an expression vector according to claim 11.

13. A method of producing an immunoglobulin molecule or fragment thereof comprising culturing a host cell according to claim 12 under conditions suitable for expression of the immunoglobulin or fragment thereof.

14. A composition comprising an immunoglobulin or fragment thereof according to claim 1 and a pharmaceutically acceptable carrier.

15. A method of treating congestive heart failure comprising administering to a subject an immunoglobulin molecule or fragment thereof comprising a region where amino acid residues corresponding to at least a portion of a complementarity determining regions (CDR) is replaced with a peptide selected from the group consisting of hBNP and hBNP mimetics.

16. A method of treating diabetes comprising administering to a subject an immunoglobulin molecule or fragment thereof comprising a region where amino acid residues corresponding to at least a portion of a complementarity determining regions (CDR) is replaced with a peptide selected from the group consisting of, GLP-1, GLP-1 mimetics, GLP-2, GLP-2 mimetics, exendin, exendin mimetics, glucagons, glucagons mimetics and PACAP-38.

17. A method of treating obesity comprising administering to a subject an immunoglobulin molecule or fragment thereof comprising a region where amino acid residues corresponding to at least a portion of a complementarity determining regions (CDR) is replaced with a peptide selected from the group consisting of, GLP-1, GLP-1 mimetics, GLP-2, GLP-2 mimetics, exendin, exendin mimetics, glucagons, glucagons mimetics and PACAP-38.

18. A method of preserving or improving beta-cell function comprising administering to a subject an immunoglobulin molecule or fragment thereof comprising a region where amino acid residues corresponding to at least a portion of a complementarity determining regions (CDR) is replaced with GLP-1.

19. A method of inducing endothelial-dependent relaxation of preconstricted pulmonary artery rings comprising administering to a subject an immunoglobulin molecule or fragment thereof comprising a region where amino acid residues corresponding to at least a portion of a complementarity determining regions (CDR) is replaced with GLP-1.

20. A method comprising administering to a subject an immunoglobulin molecule or fragment thereof comprising a region where amino acid residues corresponding to at least a portion of a complementarity determining regions (CDR) is replaced with a thiazolidinedione derivative.

21. A method as in claim 20 wherein the thiazolidinedione derivative is a peroxisome proliferator-activated receptor-γ ligand.

22. A method of regulating adiponectin expression comprising administering to a subject an immunoglobulin molecule or fragment thereof comprising a region where amino acid residues corresponding to at least a portion of a complementarity determining regions (CDR) is replaced with a thiazolidinedione derivative.
